(19) United States
(12) Patent Application Publication
(10) Pub. No.: US 2016/0280688 A1
(43) Pub. Date: Sep. 29, 2016

(54) SUBSTITUTED PHENYLALANINE DERIVATIVES

(71) Applicant: BAYER PHARMA AKTIENGESELLSCHAFT, Berlin (DE)

(72) Inventors: Ulrike RÖHN, Berlin (DE); Manuel ELLERMANN, Berlin (DE); Julia STRASSBURGER, Wuppertal (DE); Astrid WENDT, Syke (DE); Susanne RÖHRIG, Hilden (DE); Robert Alan WEBSTER, Wuppertal (DE); Martina Victoria SCHMIDT, Köln (DE); Adrian TERSTEEGEN, Wuppertal (DE); Kristin BEYER, Kensington, CA (US); Martina SCHÄFER, Berlin (DE); Anja BUCHMÜLLER, Essen (DE); Christoph GERDES, Köln (DE); Michael SPERZEL, Kierspe (DE); Steffen SANDMANN, Essen-Heisingen (DE); Stefan HEITMEIER, Wülfrath (DE); Alexander HILLISCH, Solingen (DE); Jens ACKERSTAFF, Düsseldorf (DE); Carsten TERJUNG, (US)

(73) Assignee: Bayer Pharma Aktiengesellschaft, Berlin (DE)

(21) Appl. No.: 15/024,961

(22) PCT Filed: Sep. 24, 2014
(86) PCT No.: PCT/EP2014/070324

§ 371 Date: Mar. 25, 2016

(30) Foreign Application Priority Data
Sep. 26, 2013 (EP) 13186060.3

(51) Int. Cl. C07D 403/12; C07D 401/12; C07D 413/12; C07D 471/04

(52) U.S. Cl. CPC C07D 403/12; C07D 471/04; C07D 401/12; C07D 413/12

(57) ABSTRACT

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood.

In the more recent past, the traditional theory of two separate regions of the coagulation cascade (extrinsic and intrinsic path) has been modified owing to new findings: In these models, coagulation is initiated by binding of activated factor VIIa to tissue factor (TF). The resulting complex activates factor X, which in turn leads to generation of thrombin with subsequent production of fibrin and platelet activation (via PAR-1) as injury-sealing end products of haemostasis. Compared to the subsequent amplification/propagation phase, the thrombin production rate is low and as a result of the occurrence of TFPI as inhibitor of the TF-FVIIa-FX complex is limited in time.

A central component of the transition from initiation to amplification and propagation of coagulation is factor XIa. In positive feedback loops, thrombin activates, in addition to factor V and factor VIII, also factor XI to factor XIa, whereby factor IX is converted into factor IXa, thus, via the factor IXa/factor VIIIa complex generated in this manner, rapidly producing relatively large amounts of factor Xa. This triggers the production of large amounts of thrombin, leading to strong thrombus growth and stabilizing the thrombus.

The formation of a thrombus or blood clot is counter-regulated by fibrinolysis. Activation of plasminogen by tissue plasminogen activator (tPA) results in formation of the active serine protease, plasmin, which cleaves polymerized fibrin and thus forms the thrombus. This process is referred to as fibrinolysis—with plasmin as key enzyme.

Uncontrolled activation of the coagulation system or defects in the inhibition of the activation processes may cause formation of local thromboses or embolisms in vessels (arteries, veins, lymph vessels) or heart chambers. This may lead to serious thrombotic or thromboembolic disorders. In addition, systemic hypercoagulability may lead to consumption coagulopathy in the context of a disseminated intravasal coagulation.

In the course of many cardiovascular and metabolic disorders, there is an increased tendency for coagulation and platelet activation owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis. This unwanted and excessive haemostasis may, by formation of fibrin- and platelet-rich thrombi, lead to thromboembolic disorders and thrombotic complications with life-threatening conditions.

Thromboembolic disorders are the most frequent cause of morbidity and mortality in most industrialized countries [Heart Disease: A Textbook of Cardiovascular Medicine, Eugene Braunwald, 5. edition, 1997, W.B. Saunders Company, Philadelphia].

The anticoagulants known from the prior art, for example substances for inhibiting or preventing blood coagulation, have various, frequently grave disadvantages. Accordingly, in practice, efficient treatment methods or the prophylaxis of thrombotic/thromboembolic disorders are found to be very difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopenia, alopecia medicomentosa or osteoporosis [Pschyrembel, Klinisches Worterbuch [clinical dictionary], 257th edition, 1994, Walter de Gruyter Verlag, page 610, keyword “Heparin”; Römpp Lexikon Chemie, version 1.5, 1998, Georg Thieme Verlag Stuttgart, keyword “Heparin”]. Low-molecular-weight heparins do have a lower probability of leading to the development of heparin-induced thrombocytopenia; however, they can likewise only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other cumarin derivatives which non-selectively inhibit the synthesis of various products of certain vitamin K-dependent coagulation factors in the liver. Owing to the mechanism of action, the onset of action is very slow (latency to the onset of action 36 to 48 hours). The compounds can be administered orally; however, owing to the high risk of bleeding and the narrow therapeutic index, complicated individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683]. In addition, other side-effects such as gastrointestinal problems, hair loss and skin necroses have been described.

More recent approaches for oral anticoagulants are in various phases of clinical evaluation or in clinical use, but they have also shown disadvantages, for example highly variable bioavailability, liver damage and bleeding complications.

For antithrombotic medicaments, the therapeutic width is of central importance: The distance between the therapeutically active dose for coagulation inhibition and the dose where bleeding may occur should be as big as possible so that maximum therapeutic activity is achieved at a minimum risk profile.

In various in vivo models with, for example, antibodies as factor XIa inhibitors, but also in factor XIa knock-out models, the antithrombotic effect with small/no prolongation of bleeding time or extension of blood volume was confirmed. In clinical studies, elevated factor XIa concentrations were associated with an increased event rate. However, factor XI deficiency (haemophilia C), in contrast to factor VIIIa or factor IXa (haemophilia A and B, respectively), did not lead to spontaneous bleeding and was only noticed during surgical interventions and traumata. Instead, protection against certain thromboembolic events was found.

In the event of hyperfibrinolytic states, there is inadequate wound closure, which causes severe, sometimes life-threatening, bleeding. This bleeding can be stopped by the inhibition of fibrinolysis with antifibrinolytics, by which plasmin activity is reduced. Corresponding effects with the plasminogen inhibitor tranexamic acid have been shown in various clinical studies.

It is therefore an object of the present invention to provide novel compounds for treatment and/or prophylaxis of cardiovascular disorders and/or severe perioperative blood loss in man and animals, said compounds having a wide therapeutic range.

WO89/11852 describes, inter alia, substituted phenylalanine derivatives for treatment of pancreatitis, and WO 2007/07016 describes substituted thiophene derivatives as factor XIa inhibitors.

The invention provides compounds of the formula

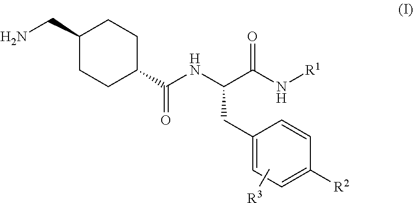

in which

R¹ is a group of the formula

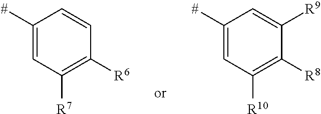

where # is the attachment site to the nitrogen atom,
R⁶ is 5-membered heteroaryl,
   where heteroaryl may be substituted by a substituent selected from the group consisting of oxo, chlorine, cyano, hydroxyl and C₁-C₃-alkyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl and methoxy, and in which alkyl is additionally substituted by 1 to 6 fluorine substituents,
R⁷ is hydrogen, fluorine or chlorine,
R⁸ and R⁹ together with the carbon atoms to which they are bonded form a 5-membered heterocycle,
   where the heterocycle may be substituted by 1 to 2 substituents selected independently from the group consisting of oxo, chlorine, cyano, hydroxyl, C₁-C₃-alkyl, pyrazolyl and pyridyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl and methoxy, and in which alkyl is additionally substituted by 1 to 6 fluorine substituents,
R¹⁰ is hydrogen, fluorine or chlorine,

R² is 9- or 10-membered bicyclic heteroaryl,
where heteroaryl may be substituted by 1 to 3 substituents selected independently from the group consisting of oxo, fluorine, chlorine, cyano, trifluoromethyl, hydroxyl, amino, C₁-C₃-alkylamino, C₁-C₃-alkyl and C₃-C₆-cycloalkyl,
   in which alkyl may be substituted by a substituent selected from the group consisting of amino and C₁-C₃-alkylamino,
or

R² is a group of the formula

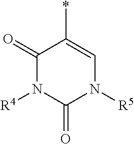

where * is the attachment site to the phenyl ring,
R⁴ is hydrogen, C₁-C₄-alkyl or benzyl,
R⁵ is hydrogen, C₁-C₄-alkyl or benzyl,

R³ is hydrogen, fluorine, chlorine, methyl or methoxy,

and the salts thereof, the solvates thereof and the solvates of the salts thereof.

Inventive compounds are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The inventive compounds may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the inventive compounds can occur in tautomeric forms, the present invention encompasses all the tautomeric forms.

The present invention also encompasses all suitable isotopic variants of the inventive compounds. An isotopic variant of an inventive compound is understood here as meaning a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I, and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore in some cases also constitute a preferred embodiment of the present invention. Isotopic variants of the inventive compounds can be prepared by the processes known to those skilled in the art, for example by the methods described below and the procedures described in the working examples, by using corresponding isotopic modifications of the respective reagents and/or starting compounds.

In the context of the present invention, preferred salts are physiologically acceptable salts of the inventive compounds. The invention also encompasses salts which themselves are unsuitable for pharmaceutical applications but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

In the context of the invention, solvates refer to those forms of the inventive compounds which, in the solid or liquid state, form a complex by coordination with solvent molecules. Hydrates are a specific form of the solvates in which the coordination is with water.

In addition, the present invention also encompasses prodrugs of the inventive compounds. The term “prodrugs” includes compounds which may themselves be biologically active or inactive but are converted to inventive compounds while resident in the body (for example metabolically or hydrolytically).

The two ways (A) and (B) of representing a 1,4-disubstituted cyclohexyl derivative shown below are equivalent to one another and identical, and in both cases describe a trans-1,4-disubstituted cyclohexyl derivative.

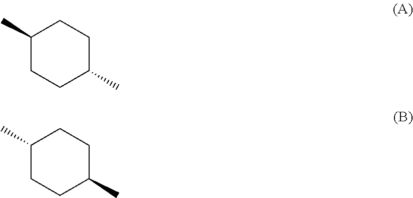

This applies especially to the structural element of tranexamamide, for example N-[(trans-4-{[(tert-butoxycarbonyl)amino]methyl}cyclohexyl)carbonyl and trans-4-(aminomethyl)-cyclohexyl]carbonyl}. In the present invention, representation (A) is used.

The three ways (C), (D) and (E) of representing tautomers of a triazole derivative shown below are equivalent to one another and identical and in all cases describe a 1,4-disubstituted triazole derivative.

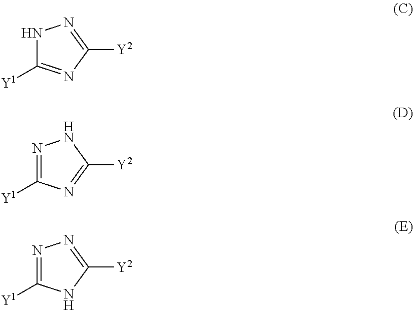

This applies especially to the following structural elements: 1H-1,2,4-triazol-3-yl, 1H-1,2,4-triazol-5-yl, 4H-1,2,4-triazol-3-yl and 4H-1,2,4-triazol-5-yl. Y¹ and Y² here are different substituents.

The two ways (F) and (G) of representing tautomers of a tetrazole derivative shown below are equivalent to one another and identical and in all cases describe a tetrazole derivative.

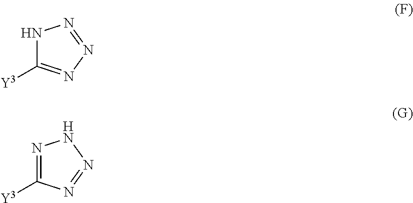

This applies especially to the following structural elements: 1H-tetrazol-5-yl and 2H-tetrazol-5-yl. Y³ here is the remainder of the compound.

The inventive compounds of the formula

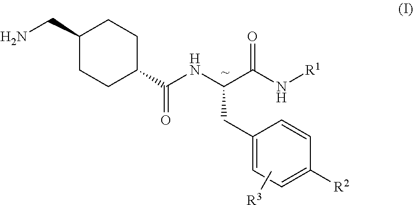

and all L-phenylalanine intermediates are described as the (S) configuration at the stereocentre marked with a “˜” in the above formulae, since L-phenylalanine derivatives are introduced into the synthesis as central units. In the preparation of the inventive compounds, the coupling of the L-phenylalanine intermediates with the amine H₂N-R¹ can result in partial epimerization at the stereocentre marked by a “˜”. Thus, a mixture of the inventive compounds of (S) enantiomer and (R) enantiomer can arise. The main component is the (S) enantiomer depicted in each case. The mixtures of (S) enantiomer and (R) enantiomer can be separated into their enantiomers by methods known to those skilled in the art, for example by chromatography on a chiral phase.

The enantiomers can be separated either directly after the coupling of the L-phenylalanine intermediates with the amine H₂N-R¹ or at a later synthesis intermediate, or else the inventive compounds can be separated themselves. Preference is given to the separation of the enantiomers directly after the coupling of the L-phenylalanine intermediates with the amine H₂N-R¹.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” or “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, unless specified otherwise, the substituents are defined as follows:

Alkyl is a linear or branched alkyl radical having 1 to 4 carbon atoms, preferably 1 to 3 carbon atoms, by way of example and with preference methyl, ethyl, n-propyl, isopropyl, 2-methylprop-1-yl, n-butyl and tert-butyl.

Alkoxy is a linear or branched alkoxy radical having 1 to 4 carbon atoms, preferably 1 to 3 carbon atoms, by way of example and with preference methoxy, ethoxy, n-propoxy, isopropoxy, 2-methylprop-1-oxy, n-butoxy and tert-butoxy.

Alkylamino is an amino group having one or two independently selected, identical or different, linear or branched alkyl radicals each having 1 to 3 carbon atoms, for example and with preference methylamino, ethylamino, n-propylamino, isopropylamino, N,N-dimethylamino, N,N-diethylamino, N-ethyl-N-methylamino, N-methyl-N-n-propylamino, N-isopropyl-N-n-propylamino and N,N-diisopropylamino. C₁-C₃-Alkylamino is, for example, a monoalkylamino radical having 1 to 3 carbon atoms or a dialkylamino radical having 1 to 3 carbon atoms in each alkyl radical.

Cycloalkyl is a monocyclic cycloalkyl group having 3 to 6 carbon atoms, preferred examples of cycloalkyl being cyclopropyl, cyclobutyl, cyclopentyl and cyclohexyl.

9- or 10-membered bicyclic heteroaryl in the definition of the R² radical is an aromatic or partly aromatic bicyclic radical having 9 or 10 ring atoms and up to 4 heteroatoms and/or hetero groups from the group of S, O, N, SO and SO₂, where one nitrogen atom may also form an N-oxide, by way of example and with preference benzimidazolyl, benzoxazolyl, benzothiazolyl, benzofuranyl, benzothiophenyl, indazolyl, pyrrolopyridinyl, quinolinyl, isoquinolinyl, dihydroquinolinyl, tetrahydroquinolinyl, quinazolyl, quinoxalinyl, 1H-imidazo[4,5-b]pyridin-6-yl, 1,2,3,4-tetrahydropyrido[2,3-b]pyrazin-7-yl, 2,3-dihydro-1H-isoindol-5-yl, 2,3-dihydro-1H-indazol-6-yl, [1,2,4]triazolo[1,5-a]pyridin-6-yl and 3H-imidazo[4,5-b]pyridin-5-yl, more preferably benzimidazolyl, indazolyl, pyrrolopyridinyl, isoquinolinyl, tetrahydroquinolinyl, 1H-imidazo[4,5-b]pyridin-6-yl, 1,2,3,4-tetrahydropyrido[2,3-b]pyrazin-7-yl, 2,3-dihydro-1H-isoindol-5-yl, 2,3-dihydro-1H-indazol-6-yl, [1,2,4]triazolo[1,5-a]pyridin-6-yl and 3H-imidazo[4,5-b]pyridin-5-yl.

5-membered heteroaryl in the definition of the R⁶ radical is an aromatic monocyclic radical having 5 ring atoms and up to 4 heteroatoms and/or hetero groups from the group of S, O, N, SO and SO₂, where one nitrogen atom may also form an N-oxide, for example and with preference thienyl, furyl, pyrrolyl, thiazolyl, oxazolyl, isoxazolyl, oxadiazolyl, pyrazolyl, imidazolyl, triazolyl and tetrazolyl, more preferably triazolyl and tetrazolyl, most preferably tetrazolyl.

5-membered heterocycle in the definition of the R⁸ and R⁹ radicals is a saturated, partly unsaturated or aromatic monocyclic radical having 5 ring atoms and up to 2 heteroatoms and/or hetero groups from the group of S, O, N, SO and SO₂, where one nitrogen atom may also form an N-oxide. This 5-membered heterocycle together with the phenyl ring to which it is bonded is, for example and with preference, 2,3-dihydro-1-benzothiophen-5-yl, 1,3-dihydro-2-benzothiophen-5-yl, 2,3-dihydro-1-benzofuran-5-yl, 1,3-dihydro-2-benzofuran-5-yl, indolin-5-yl, isoindolin-5-yl, 2,3-dihydro-1H-indazol-5-yl, 2,3-dihydro-1H-benzimidazol-5-yl, 1,3-dihydro-2,1-benzoxazol-5-yl, 2,3-dihydro-1,3-benzoxazol-5-yl, 1,3-dihydro-2,1-benzothiazol-5-yl, 2,3-dihydro-1,3-benzothiazol-5-yl, 1H-benzimidazol-5-yl, 1H-indazol-5-yl, 1,2-benzoxazol-5-yl, indol-5-yl, isoindol-5-yl, benzofuran-5-yl, benzothiophen-5-yl, 2,3-dihydro-1-benzothiophen-6-yl, 1,3-dihydro-2-benzothiophen-6-yl, 2,3-dihydro-1-benzofuran-6-yl, 1,3-dihydro-2-benzofuran-6-yl, indolin-6-yl, isoindolin-6-yl, 2,3-dihydro-1H-indazol-6-yl, 2,3-dihydro-1H-benzimidazol-6-yl, 1,3-dihydro-2,1-benzoxazol-6-yl, 2,3-dihydro-1,3-benzoxazol-6-yl, 1,3-dihydro-2,1-benzothiazol-6-yl, 2,3-dihydro-1,3-benzothiazol-6-yl, 1H-benzimidazol-6-yl, 1H-indazol-6-yl, 1,2-benzoxazol-6-yl, indol-6-yl, isoindol-6-yl, benzofuran-6-yl and benzothiophen-6-yl, more preferably 2,3-dihydro-1H-benzimidazol-5-yl, 1H-benzimidazol-6-yl, 2,3-dihydro-1H-indazol-6-yl and 1H-indazol-6-yl, most preferably 2,3-dihydro-1H-benzimidazol-5-yl and 2,3-dihydro-1H-indazol-6-yl.

In the formulae of the group which may represent R′, the end point of the line marked by # does not represent a carbon atom or a CH₂ group, but is part of the bond to the atom to which R¹ is bonded.

In the formulae of the group which may represent R², the end point of the line marked by * does not represent a carbon atom or a CH₂ group, but is part of the bond to the atom to which R² is bonded.

Preference is given to compounds of the formula (I) in which

R¹ is a group of the formula

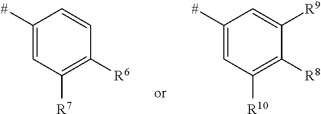

where # is the attachment site to the nitrogen atom,
R⁶ is 5-membered heteroaryl,
   where heteroaryl may be substituted by a substituent selected from the group consisting of oxo, chlorine, cyano, hydroxyl and C₁-C₃-alkyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl and methoxy, and in which alkyl is additionally substituted by 1 to 6 fluorine substituents,
R⁷ is hydrogen, fluorine or chlorine,
R⁸ and R⁹ together with the carbon atoms to which they are bonded form a 5-membered heterocycle,
   where the heterocycle may be substituted by 1 to 2 substituents selected independently from the group consisting of oxo, chlorine, cyano, hydroxyl, C₁-C₃-alkyl, pyrazolyl and pyridyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl and methoxy, and in which alkyl is additionally substituted by 1 to 6 fluorine substituents,
R¹⁰ is hydrogen, fluorine or chlorine,

R² is 9- or 10-membered bicyclic heteroaryl,
where heteroaryl may be substituted by 1 to 3 substituents selected independently from the group consisting of oxo, fluorine, chlorine, cyano, trifluoromethyl, hydroxyl, amino, C₁-C₃-alkylamino and C₁-C₃-alkyl,
   in which alkyl may be substituted by a substituent selected from the group consisting of amino and C₁-C₃-alkylamino,
or

R² is a group of the formula

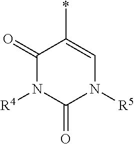

where * is the attachment site to the phenyl ring,
R⁴ is hydrogen, C₁-C₄-alkyl or benzyl,
R⁵ is hydrogen, C₁-C₄-alkyl or benzyl,

R³ is hydrogen, fluorine, chlorine, methyl or methoxy,

and the salts thereof, the solvates thereof and the solvates of the salts thereof.

Preference is also given to compounds of the formula (I) in which

R¹ is a group of the formula

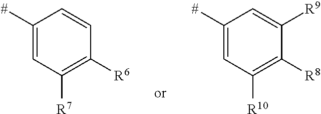

where # is the attachment site to the nitrogen atom,
R⁶ is 5-membered heteroaryl,
   where heteroaryl may be substituted by a substituent selected from the group consisting of oxo, chlorine and C₁-C₃-alkyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁷ is hydrogen or fluorine,
R⁸ and R⁹ together with the carbon atoms to which they are bonded form a 5-membered heterocycle,
   where the heterocycle may be substituted by 1 to 2 substituents selected independently from the group consisting of oxo, chlorine, hydroxyl, C₁-C₃-alkyl, pyrazolyl and pyridyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R¹⁰ is hydrogen or fluorine,

R² is 9- or 10-membered bicyclic heteroaryl,
where heteroaryl may be substituted by 1 to 3 substituents selected independently from the group consisting of oxo, fluorine, chlorine, hydroxyl, amino, C₁-C₃-alkylamino and C₁-C₃-alkyl,
   in which alkyl may be substituted by a substituent selected from the group consisting of amino and C₁-C₃-alkylamino,
or

R² is a group of the formula

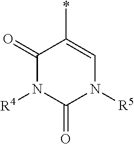

where * is the attachment site to the phenyl ring,
R⁴ is hydrogen, methyl or benzyl,
R⁵ is hydrogen, methyl or benzyl,

R³ is hydrogen, fluorine, methyl or methoxy,

and the salts thereof, the solvates thereof and the solvates of the salts thereof.

Preference is also given to compounds of the formula (I) in which

R¹ is a group of the formula

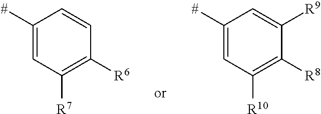

where # is the attachment site to the nitrogen atom,
R⁶ is 5-membered heteroaryl,
R⁷ is hydrogen or fluorine,
R⁸ and R⁹ together with the carbon atoms to which they are bonded form a 5-membered heterocycle,
   where the heterocycle may be substituted by an oxo substituent,
R¹⁰ is hydrogen,

R² is 9- or 10-membered bicyclic heteroaryl,
where heteroaryl may be substituted by 1 to 2 substituents selected independently from the group consisting of oxo, amino and C₁-C₃-alkyl,
   in which alkyl may be substituted by an amino substituent,
or

R² is a group of the formula

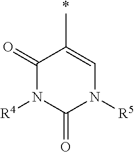

where * is the attachment site to the phenyl ring,
R⁴ is hydrogen or methyl,
R⁵ is hydrogen, methyl or benzyl,

R³ is hydrogen,

and the salts thereof, the solvates thereof and the solvates of the salts thereof.

Preference is also given to compounds of the formula (I) in which

R¹ is a group of the formula

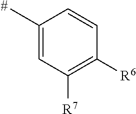

where # is the attachment site to the nitrogen atom,
R⁶ is tetrazolyl,
R⁷ is hydrogen or fluorine,

or

R¹ is 2,3-dihydro-1H-indazol-6-yl,
where 2,3-dihydro-1H-indazol-6-yl may be substituted by an oxo substituent,

R² is benzimidazolyl, indazolyl, pyrrolopyridinyl, isoquinolinyl or tetrahydroquinolinyl,
where benzimidazolyl, indazolyl, pyrrolopyridinyl, isoquinolinyl and tetrahydroquinolinyl may be substituted by 1 to 2 substituents selected independently from the group consisting of oxo, amino, methyl, ethyl, n-propyl and isopropyl,
   in which ethyl, n-propyl and isopropyl may be substituted by an amino substituent,
or

R² is a group of the formula

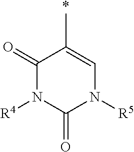

where * is the attachment site to the phenyl ring,
R⁴ is hydrogen or methyl,
R⁵ is hydrogen, methyl or benzyl,

R³ is hydrogen,

and the salts thereof, the solvates thereof and the solvates of the salts thereof.

Preference is also given to compounds of the formula (I) in which

R¹ is a group of the formula

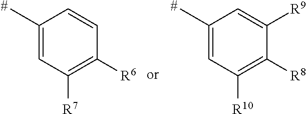

where # is the attachment site to the nitrogen atom,
R⁶ is 5-membered heteroaryl,
   where heteroaryl may be substituted by a substituent selected from the group consisting of oxo, chlorine and C₁-C₃-alkyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁷ is hydrogen or fluorine,
R⁸ and R⁹ together with the carbon atoms to which they are bonded form a 5-membered heterocycle,
   where the heterocycle may be substituted by 1 to 2 substituents selected independently from the group consisting of oxo, chlorine, hydroxyl, C₁-C₃-alkyl, pyrazolyl and pyridyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R¹⁰ is hydrogen or fluorine,

R² is 9- or 10-membered bicyclic heteroaryl,
where heteroaryl may be substituted by 1 to 3 substituents selected independently from the group consisting of oxo, fluorine, chlorine, hydroxyl, amino, C₁-C₃-alkylamino, C₁-C₃-alkyl and C₃-C₆-cycloalkyl,
   in which alkyl may be substituted by a substituent selected from the group consisting of amino and C₁-C₃-alkylamino,
or

R² is a group of the formula

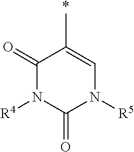

where * is the attachment site to the phenyl ring,
R⁴ is hydrogen, methyl or benzyl,
R⁵ is hydrogen, methyl or benzyl,

R³ is hydrogen, fluorine, methyl or methoxy,

and the salts thereof, the solvates thereof and the solvates of the salts thereof.

Preference is also given to compounds of the formula (I) in which

R¹ is a group of the formula

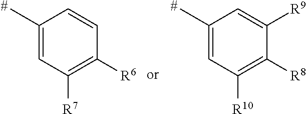

where # is the attachment site to the nitrogen atom,
R⁶ is 5-membered heteroaryl,
R⁷ is hydrogen or fluorine,
R⁸ and R⁹ together with the carbon atoms to which they are bonded form a 5-membered heterocycle,
   where the heterocycle may be substituted by an oxo substituent,
R¹⁰ is hydrogen,

R² is 9- or 10-membered bicyclic heteroaryl,
where heteroaryl may be substituted by 1 to 2 substituents selected independently from the group consisting of oxo, amino, C₁-C₃-alkyl, cyclopropyl and cyclobutyl,
   in which alkyl may be substituted by an amino substituent,

R³ is hydrogen,

and the salts thereof, the solvates thereof and the solvates of the salts thereof.

Preference is also given to compounds of the formula (I) in which

R¹ is a group of the formula

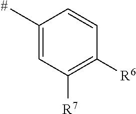

where # is the attachment site to the nitrogen atom,
R⁶ is tetrazolyl,
R⁷ is hydrogen or fluorine,

or

R¹ is 2,3-dihydro-1H-indazol-6-yl, 2,3-dihydro-1H-benzimidazol-5-yl, 1H-benzimidazol-6-yl or 1H-indazol-6-yl,
where 2,3-dihydro-1H-indazol-6-yl and 2,3-dihydro-1H-benzimidazol-5-yl may be substituted by an oxo substituent,

R² is benzimidazolyl, indazolyl, pyrrolopyridinyl, isoquinolinyl, tetrahydroquinolinyl, 1H-imidazo[4,5-b]pyridin-6-yl, 1,2,3,4-tetrahydropyrido[2,3-b]pyrazin-7-yl, 2,3-dihydro-1H-isoindol-5-yl, 2,3-dihydro-1H-indazol-6-yl, [1,2,4]triazolo-[1,5-a]pyridin-6-yl or 3H-imidazo[4,5-b]pyridin-5-yl,
where benzimidazolyl, indazolyl, pyrrolopyridinyl, isoquinolinyl, tetrahydroquinolinyl, 1H-imidazo[4,5-b]pyridin-6-yl, 1,2,3,4-tetrahydropyrido[2,3-b]pyrazin-7-yl, 2,3-dihydro-1H-isoindol-5-yl, 2,3-dihydro-1H-indazol-6-yl, [1,2,4]triazolo-[1,5-a]pyridin-6-yl and 3H-imidazo[4,5-b]pyridin-5-yl may be substituted by 1 to 2 substituents selected independently from the group consisting of oxo, amino, methyl, ethyl, n-propyl, isopropyl, cyclopropyl and cyclobutyl,
   in which ethyl, n-propyl and isopropyl may be substituted by an amino substituent,

R³ is hydrogen,

and the salts thereof, the solvates thereof and the solvates of the salts thereof.

Preference is also given to compounds of the formula (I) in which

R¹ is a group of the formula

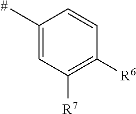

where # is the attachment site to the nitrogen atom,
R⁶ is tetrazolyl,
and
R⁷ is hydrogen or fluorine.

Preference is also given to compounds of the formula (I) in which

R¹ is 2,3-dihydro-1H-indazol-6-yl,
where 2,3-dihydro-1H-indazol-6-yl may be substituted by an oxo substituent.

Preference is also given to compounds of the formula (I) in which

R¹ is 2,3-dihydro-1H-indazol-6-yl, 2,3-dihydro-1H-benzimidazol-5-yl, 1H-benzimidazol-6-yl or 1H-indazol-6-yl,
where 2,3-dihydro-1H-indazol-6-yl and 2,3-dihydro-1H-benzimidazol-5-yl may be substituted by an oxo substituent.

Preference is also given to compounds of the formula (I) in which

R² is benzimidazolyl, indazolyl, pyrrolopyridinyl, isoquinolinyl or tetrahydroquinolinyl,
where benzimidazolyl, indazolyl, pyrrolopyridinyl, isoquinolinyl and tetrahydroquinolinyl may be substituted by 1 to 2 substituents selected independently from the group consisting of oxo, amino, methyl, ethyl, n-propyl and isopropyl,
   in which ethyl, n-propyl and isopropyl may be substituted by an amino substituent.

Preference is also given to compounds of the formula (I) in which

R² is benzimidazolyl, indazolyl, pyrrolopyridinyl, isoquinolinyl, tetrahydroquinolinyl, 1H-imidazo[4,5-b]pyridin-6-yl, 1,2,3,4-tetrahydropyrido[2,3-b]pyrazin-7-yl, 2,3-dihydro-1H-isoindol-5-yl, 2,3-dihydro-1H-indazol-6-yl, [1,2,4]triazolo-[1,5-a]pyridin-6-yl or 3H-imidazo[4,5-b]pyridin-5-yl,
where benzimidazolyl, indazolyl, pyrrolopyridinyl, isoquinolinyl, tetrahydroquinolinyl, 1H-imidazo[4,5-b]pyridin-6-yl, 1,2,3,4-tetrahydropyrido[2,3-b]pyrazin-7-yl, 2,3-dihydro-1H-isoindol-5-yl, 2,3-dihydro-1H-indazol-6-yl, [1,2,4]triazolo-[1,5-a]pyridin-6-yl and 3H-imidazo[4,5-b]pyridin-5-yl may be substituted by 1 to 2 substituents selected independently from the group consisting of oxo, amino, methyl, ethyl, n-propyl, isopropyl, cyclopropyl and cyclobutyl,
   in which ethyl, n-propyl and isopropyl may be substituted by an amino substituent.

Preference is also given to compounds of the formula (I) in which

R² is a group of the formula

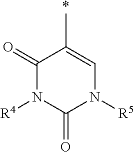

where * is the attachment site to the phenyl ring,
R⁴ is hydrogen or methyl,
and
R⁵ is hydrogen, methyl or benzyl.

Preference is also given to compounds of the formula (I) in which R³ is hydrogen.

The individual radical definitions specified in the particular combinations or preferred combinations of radicals are, independently of the particular combinations of the radicals specified, also replaced as desired by radical definitions of other combinations.

Very particular preference is given to combinations of two or more of the abovementioned preferred ranges.

The invention further provides a process for preparing the compounds of the formula (I), or the salts thereof, solvates thereof and the solvates of the salts thereof, wherein the compounds of the formula

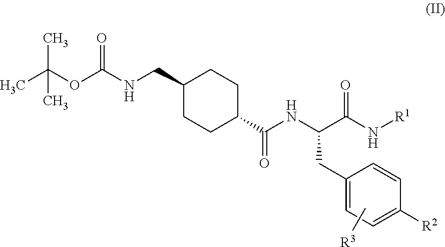

in which

R¹, R² and R³ are each as defined above,

are reacted with an acid.

The reaction is generally effected in inert solvents, preferably within a temperature range from room temperature to 60° C. at standard pressure.

Inert solvents are, for example, halogenated hydrocarbons such as dichloromethane, trichloromethane, carbon tetrachloride or 1,2-dichloroethane, or ethers such as tetrahydrofuran or dioxane, preference being given to dioxane.

Acids are, for example, trifluoroacetic acid or hydrogen chloride in dioxane, preference being given to hydrogen chloride in dioxane.

The compounds of the formula (II) are known or can be prepared by reacting

[A] Compounds of the Formula

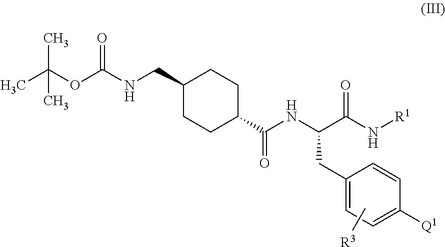

in which

R¹ and R³ are each as defined above, and

Q¹ is —B(OH)₂, a boronic ester, preferably pinacol boronate, or —BF₃⁻K⁺,

with compounds of the formula

X¹—R² (IV)

in which

R² is as defined above, and

X¹ is bromine or iodine,

under Suzuki coupling conditions,

or

[B] Compounds of the Formula

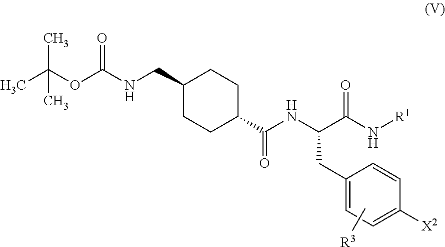

in which

R¹ and R³ are each as defined above, and

X² is bromine or iodine,

with compounds of the formula

Q²-R² (VI)

in which

R² is as defined above, and

Q² is —B(OH)₂, a boronic ester, preferably pinacol boronate, or —BF₃⁻K⁺,

under Suzuki coupling conditions,

or

[C] Compounds of the Formula

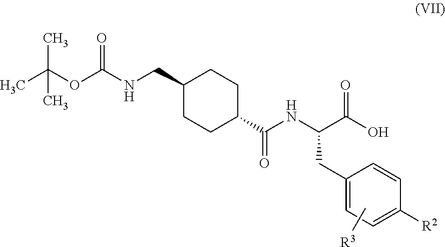

in which

R² and R³ are each as defined above,

with compounds of the formula

H₂N—R¹ (VIII)

in which

R¹ is as defined above,

in the presence of dehydrating reagents.

The reaction in process [A] is generally effected in inert solvents, in the presence of a catalyst, optionally in the presence of an additional reagent, optionally in a microwave, preferably within a temperature range from room temperature to 150° C. at standard pressure to 3 bar.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphineferrocenyl)palladium(II) chloride, 1,3-bis(2,6-diisopropylphenyl)imidazol-2-ylidene(1,4-napththoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)], preference being given to tetrakistriphenylphosphinepalladium(0), [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)].

Additional reagents are, for example, potassium acetate, caesium carbonate, potassium carbonate or sodium carbonate, potassium tert-butoxide, caesium fluoride or potassium phosphate, which may be present in aqueous solution; preferred additional reagents are those such as potassium acetate or a mixture of potassium acetate and sodium carbonate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, oder N-methylpyrrolidone or acetonitrile, or mixtures of the solvents with alcohols such as methanol or ethanol and/or water, preference being given to toluene, dimethylformamide or dimethyl sulphoxide.

The compounds of the formula (IV) are known, can be synthesized from the corresponding starting compounds by known processes or can be prepared analogously to the processes described in the Examples section.

The reaction in process [B] is effected as described for process [A].

The compounds of the formula (VI) are known, can be synthesized from the corresponding starting compounds by known processes or can be prepared analogously to the processes described in the Examples section.

The reaction in process [C] is generally effected in inert solvents, optionally in the presence of a base, preferably within a temperature range from 0° C. to the reflux of the solvents at standard pressure.

Suitable dehydrating reagents here are, for example, carbodiimides, for example N,N′-diethyl-, N,N′-dipropyl-, N,N′-diisopropyl- and N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimid-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl) -1,1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or ethyl cyano(hydroxyimino)acetate (Oxyma), or (1-cyano-2-ethoxy-2-oxoethylideneaminooxy)dimethylaminomorpholinocarbenium hexafluorophosphate (COMU), or N-[(dimethylamino)(3H-[1,2,3]triazolo[4,5-b]pyridin-3-yloxy)methylidene]-N-methylmethanaminium hexafluorophosphate, or 2,4,6-tripropyl-1,3,5,2,4,6-trioxatriphosphinane 2,4,6-trioxide (T3P), or mixtures of these, preference being given to N-[(dimethylamino)(3H-[1,2,3]triazolo[4,5-b]pyridin-3-yloxy)methylidene]-N-methylmethanaminium hexafluorophosphate or 2,4,6-tripropyl-1,3,5,2,4,6-trioxatriphosphinane 2,4,6-trioxide (T3P).

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine, preference being given to diisopropylethylamine

Inert solvents are, for example, halogenated hydrocarbons such as dichloromethane or trichloromethane, hydrocarbons such as benzene, or other solvents such as nitromethane, tetrahydrofuran, dioxane, dimethylformamide, dimethyl sulphoxide, acetonitrile or pyridine, or mixtures of the solvents, preference being given to tetrahydrofuran or dimethylformamide or a mixture of dimethylformamide and pyridine.

The compounds of the formula (VIII) are known, can be synthesized from the corresponding starting compounds by known processes or can be prepared analogously to the processes described in the Examples section.

The compounds of the formula (III) are known or can be prepared by reacting compounds of the formula (V) with 4,4,4′,4′,5,5,5′,5′-octamethyl-2,2′-bi-1,3,2-dioxaborolane.

The reaction is generally effected in inert solvents, in the presence of a catalyst, optionally in the presence of an additional reagent, optionally in a microwave, preferably within a temperature range from room temperature to 150° C. at standard pressure to 3 bar. Hydrolysis in an acidic medium affords the corresponding boronic acids. Workup with potassium hydrogendifluoride solution (KHF₂ solution) affords the corresponding trifluoroborates.

Catalysts are, for example, palladium catalysts customary for the borylation of aryl halides, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphineferrocenyl)palladium(II) chloride, 1,3-bis(2,6-diisopropylphenyl)imidazol-2-ylidene(1,4-napththoquinone)palladium dimer, allyl(chloro)(1,3-dimesityl -1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)], preference being given to tetrakistriphenylphosphinepalladium(0) and [1,1-bis(diphenylphosphino)ferrocene]palladium(II) chloride.

Additional reagents are, for example, potassium acetate, caesium carbonate, potassium carbonate or sodium carbonate, potassium tert-butoxide or sodium tert-butoxide, caesium fluoride, potassium phosphate or potassium phenoxide, preference being given to potassium acetate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, preference being given to dioxane, dimethylformamide or dimethyl sulphoxide.

Literature: K. L. Billingslay, T. E. Barde, S. L Buchwald, Angew. Chem. 2007, 119, 5455 or T. Graening, Nachrichten aus der Chemie, January 2009, 57, 34.

The compounds of the formula (V) are known or can be prepared by reacting compounds of the formula

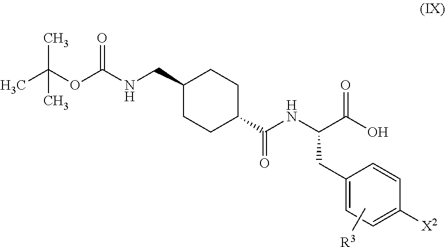

in which

R³ is as defined above, and

X² is bromine or iodine,

with compounds of the formula (VIII) in the presence of dehydrating reagents.

The reaction is carried out as described for process [C].

The compounds of the formula (IX) are known, can be synthesized from the corresponding starting compounds by known processes or can be prepared analogously to the processes described in the Examples section.

The compounds of the formula (VII) are known or can be prepared by reacting compounds of the formula

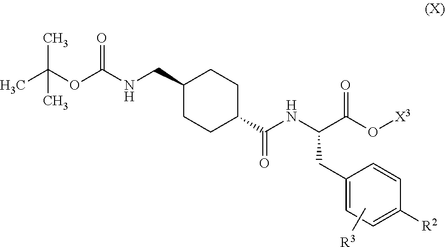

in which

R² and R³ are each as defined above, and

X³ is methyl or ethyl,

with a base.

The reaction is generally carried out in inert solvents, preferably in a temperature range of from room temperature to reflux of the solvents at atmospheric pressure.

Inert solvents are, for example, halogenated hydrocarbons such as dichloromethane, trichloromethane, carbon tetrachloride or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvents with water, preference being given to a mixture of tetrahydrofuran and water.

Bases are, for example, alkali metal hydroxides such as sodium hydroxide, lithium hydroxide or potassium hydroxide, or alkali metal carbonates such as caesium carbonate, sodium carbonate or potassium carbonate, or alkoxides such as potassium tert-butoxide or sodium tert-butoxide, preference being given to sodium hydroxide and lithium hydroxide.

The compounds of the formula (X) are known or can be prepared by reacting compounds of the formula

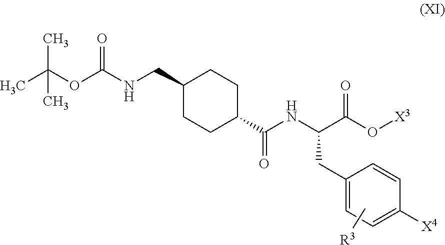

in which

R³ is as defined above,

X³ is methyl or ethyl, and

X⁴ is bromine or iodine,

with compounds of the formula (VI) under Suzuki coupling conditions.

The reaction is effected as described for process [A].

The compounds of the formula (XI) are known, can be synthesized from the corresponding starting compounds by known processes or can be prepared analogously to the processes described in the Examples section.

The preparation of the starting compounds and of the compounds of the formula (I) can be illustrated by the synthesis scheme below.

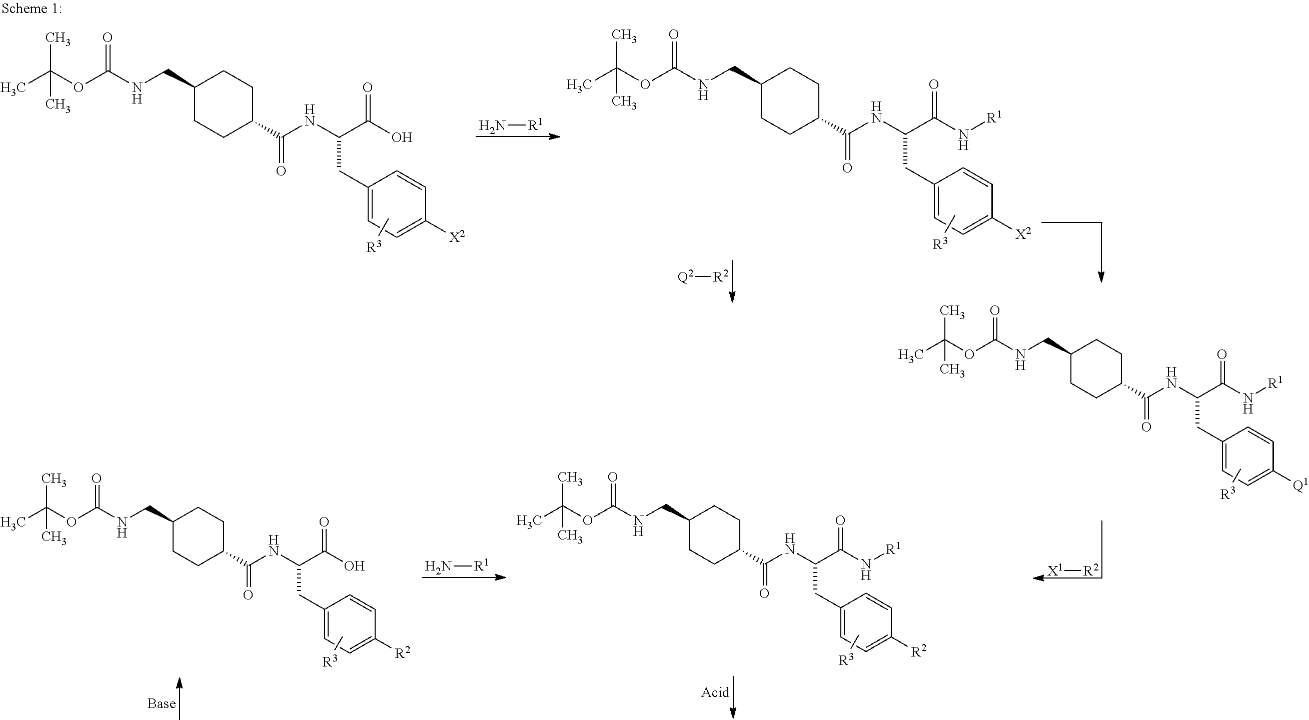

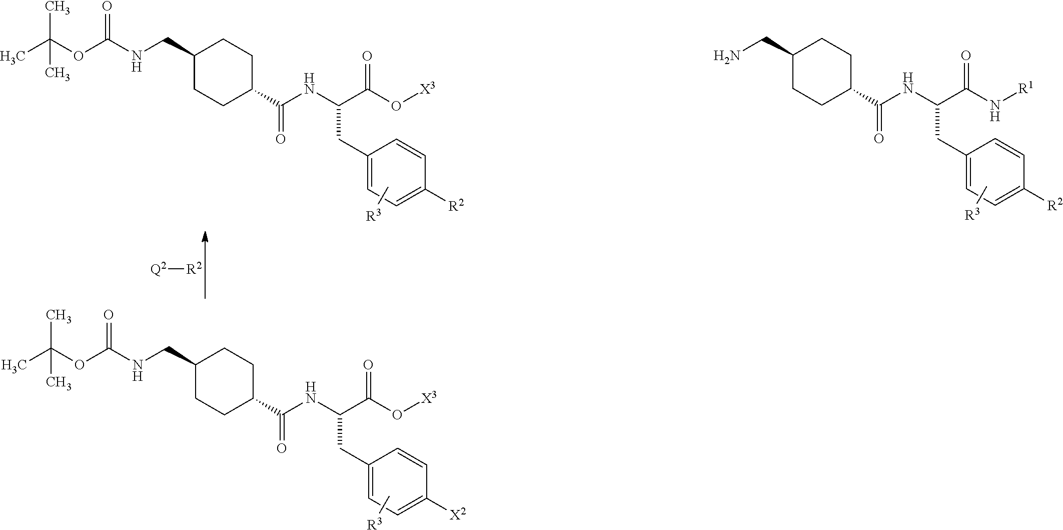

The inventive compounds have an unforeseeable useful spectrum of pharmacological activity and good pharmacokinetic properties. They are compounds that influence the proteolytic activity of the serine proteases FXIa and kallikrein, and possibly plasmin. The inventive compounds inhibit the enzymatic cleavage of substrates that assume a major role in the activation of the blood coagulation cascade and platelet aggregation. If the inventive compounds inhibit plasmin activity, the result is inhibition of fibrinolysis.

They are therefore suitable for use as medicaments for treatment and/or prophylaxis of diseases in man and animals

The present invention further provides for the use of the inventive compounds for treatment and/or prophylaxis of disorders, especially cardiovascular disorders, preferably thrombotic or thromboembolic disorders and/or thrombotic or thromboembolic complications.

“Thromboembolic disorders” in the sense of the present invention include in particular disorders such as acute coronary syndrome (ACS), ST-segment elevation myocardial infarction (STEMI) and non-ST-segment elevation myocardial infarction (non-STEMI), stable angina pectoris, unstable angina pectoris, reocclusions and restenoses after coronary interventions such as angioplasty, stent implantation or aortocoronary bypass, peripheral arterial occlusion diseases, pulmonary embolisms, venous thromboses, especially in deep leg veins and renal veins, transitory ischaemic attacks and also thrombotic and thromboembolic stroke.

The inventive compounds are therefore also suitable for the prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with artificial heart valves.

In addition, the inventive compounds are suitable for the treatment and prevention of disseminated intravascular coagulation (DIC) which may occur in connection with sepsis inter alfa, but also owing to surgical interventions, neoplastic disorders, burns or other injuries and may lead to severe organ damage through microthrombosis.

Thromboembolic complications are also encountered in microangiopathic haemolytic anaemias, extracorporeal circulatory systems, such as haemodialysis, and also prosthetic heart valves.

In addition, the inventive compounds are also used for influencing wound healing, for the prophylaxis and/or treatment of atherosclerotic vascular disorders and inflammatory disorders, such as rheumatic disorders of the locomotive system, coronary heart diseases, of heart failure, of hypertension, of inflammatory disorders, for example asthma, inflammatory pulmonary disorders, glomerulonephritis and inflammatory intestinal disorders, for example Crohn's disease or ulcerative colitis or acute renal failure, and additionally likewise for the prophylaxis and/or treatment of dementia disorders, for example Alzheimer's disease. In addition, the inventive compounds can be used for inhibiting tumour growth and the formation of metastases, for microangiopathies, age-related macular degeneration, diabetic retinopathy, diabetic nephropathy and other microvascular disorders, and also for the prevention and treatment of thromboembolic complications, for example venous thromboembolisms, for tumour patients, especially those undergoing major surgery or chemo- or radiotherapy.

In addition, the inventive compounds are also suitable for the prophylaxis and/or treatment of pulmonary hypertension.

The term “pulmonary hypertension” includes certain forms of pulmonary hypertension, as determined, for example, by the World Health Organization (WHO). Examples include pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hyptertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for treatment and/or prophylaxis of pulmonary hypertension associated with sarcoidosis, histiocytosis X and lymphangiomatosis.

In addition, the inventive substances may also be useful for treatment of pulmonary and hepatic fibroses.

In addition, the inventive compounds may also be suitable for treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications. Moreover, there may be endothelial damage with increased permeability of the vessels and seeping of fluids and proteins into the extravasal lumen. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or injured extravascular tissue. As a consequence, there is coagulation in small vessels of various organs with hypoxia and subsequent organ dysfunction. This can be prevented by the inventive compounds. A secondary effect is the consumption of coagulation factors (for example factor X, prothrombin and fibrinogen) and platelets, which reduces the coagulability of the blood and may result in heavy bleeding.

In addition, the inventive compounds are also useful for the prophylaxis and/or treatment of hyperfibrinolysis. The prophylaxis and/or treatment may reduce or eliminate severe perioperative blood loss. Severe bleeding occurs in major operations, for example coronary artery bypass surgery, transplants or hysterectomy, and in the event of trauma, in the event of haemorrhagic shock or in the event of postpartum haemorrhage. In the aforementioned indications, there may be perioperative use of extracorporeal circulation systems or filter systems, for example heart and lung machines, haemofiltration, haemodialysis, extracorporeal membrane oxygenation or a ventricular support system, for example artificial heart. This additionally requires anticoagulation, for which the inventive compounds can also be used.

The inventive compounds are also suitable for anticoagulation during kidney replacement procedures, for example in the case of continuous veno-venous haemofiltration or intermittent haemodialysis.

The inventive compounds can additionally also be used for preventing coagulation ex vivo, for example for preserving blood and plasma products, for cleaning/pretreating catheters and other medical auxiliaries and instruments, for coating synthetic surfaces of medical auxiliaries and instruments used in vivo or ex vivo or for biological samples which could contain factor XIa.

The present invention further provides for the use of the inventive compounds for treatment and/or prophylaxis of disorders, especially the disorders mentioned above.

The present invention further provides for the use of the inventive compounds for production of a medicament for treatment and/or prophylaxis of disorders, especially of the aforementioned disorders.

The present invention further provides a method for treatment and/or prophylaxis of disorders, especially the disorders mentioned above, using a therapeutically effective amount of an inventive compound.

The present invention further provides the inventive compounds for use in a method for treatment and/or prophylaxis of disorders, especially of the aforementioned disorders, using a therapeutically effective amount of an inventive compound.

The present invention further provides medicaments comprising an inventive compound and one or more further active ingredients.

The present invention further provides a method for preventing the coagulation of blood in vitro, especially in banked blood or biological samples which could contain factor XIa, which is characterized in that an anticoagulatory amount of the inventive compound is added.

The present invention further provides medicaments comprising an inventive compound and one or more further active ingredients, especially for treatment and/or prophylaxis of the disorders mentioned above. Preferred examples of active ingredients suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilatators, especially ACE (angiotensin converting enzyme) inhibitors, for example captopril, lisinopril, enalapril, ramipril, cilazapril, benazepril, fosinopril, quinapril and perindopril, or All (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors), for example tissue plasminogen activator (t-PA), streptokinase, reteplase and urokinase;
anticoagulatory substances (anticoagulants), for example heparin (UFH), low molecular weight heparins (LMWH), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI), for example Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux,
platelet aggregation-inhibiting substances (platelet aggregation inhibitors, thrombocyte aggregation inhibitors), for example acetylsalicylic acid (for example Aspirin), ticlopidine (Ticlid), clopidogrel (Plavix), prasugrel, ticagrelor, cangrelor, elinogrel, vorapaxar;
fibrinogen receptor antagonists (glycoprotein-IIb/IIIa antagonists), for example abciximab, eptifibatide, tirofiban, lamifiban, lefradafiban and fradafiban;
and also antiarrhythmics;
various antibiotics or antifungal medicaments, either as calculated therapy (prior to the presence of the microbial diagnosis) or as specific therapy;
vasopressors, for example norepinephrine, dopamine and vasopressin;
inotropic therapy, for example dobutamine;
corticosteroids, for example hydrocortisone and fludrocortisone;
recombinant human activated protein C, for example Xigris;
blood products, for example erythrocyte concentrates, thrombocyte concentrates, erythropietin and fresh frozen plasma.

“Combinations” for the purpose of the invention mean not only dosage forms which contain all the components (so-called fixed combinations) and combination packs which contain the components separate from one another, but also components which are administered simultaneously or sequentially, provided that they are used for prophylaxis and/or treatment of the same disease. It is likewise possible to combine two or more active ingredients with one another, meaning that they are thus each in two-component or multicomponent combinations.

The inventive compounds may act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The inventive compounds can be administered in suitable administration forms for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay and control the release of the inventive compound), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of an absorption step (for example by an intravenous, intraarterial, intracardiac, intraspinal or intralumbar route) or with inclusion of an absorption (for example by an intramuscular, subcutaneous, intracutaneous, percutaneous or intraperitoneal route). Suitable administration forms for parenteral administration include injection and infusion formulations in the form of solutions, suspensions, emulsions, lyophilizates or sterile powders.

Parenteral administration is preferred.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be accomplished in a manner known per se by mixing with inert, non-toxic, pharmaceutically suitable auxiliaries. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

In spite of this, it may be necessary to deviate from the amounts specified, specifically depending on body weight, administration route, individual behaviour towards the active ingredient, type of formulation, and time or interval of administration.

Unless stated otherwise, the percentages in the tests and examples which follow are percentages by weight; parts are parts by weight. Solvent ratios, dilution ratios and concentration data for the liquid/liquid solutions are in each case based on volume. “w/v” means “weight/volume”. For example, “10% w/v” means: 100 ml of solution or suspension comprise 10 g of substance.

### A) EXAMPLES

Abbreviations
bs/br. s. broad singlet (in NMR)
bd broad doublet (in NMR)
cat. catalytic
CI chemical ionization (in MS)
dd doublet of doublets (in NMR)
DMF dimethylformamide
DMSO dimethyl sulphoxide
dt doublet of triplets (in NMR)
of th. of theory (in yield)
EI electron impact ionization (in MS)
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure high-performance liquid chromatography
LC-MS liquid chromatography-coupled mass spectroscopy
m multiplet (in NMR)
M molar
min minute(s)
MS mass spectrometry
N normal
NMR nuclear magnetic resonance spectrometry
q quartet (in NMR)
quant. quantitative
quint quintet (in NMR)
RT room temperature
Rₜ retention time (in HPLC)
s singlet (in NMR)
TFA trifluoroacetic acid
THF tetrahydrofuran
UV ultraviolet spectrometry

HPLC and LC/MS Methods:

Method 1 (LC-MS): Instrument: Waters ACQUITY SQD UPLC System; column: Waters Acquity UPLC HSS T3 1.8μ 50×1 mm; eluent A: 1 1 water+0.25 ml 99% formic acid, eluent B: 1 1 acetonitrile+0.25 ml 99% formic acid; gradient: 0.0 min 90% A→1.2 min 5% A→2.0 min 5% A; oven: 50° C.; flow rate: 0.40 ml/min; UV detection: 210-400 nm.

Method 2 (LC-MS): Instrument: Micromass Quattro Premier with Waters UPLC Acquity; column: Thermo Hypersil GOLD 1.9μ 50 mm×1 mm; eluent A: 1 1 water+0.5 ml 50% formic acid, eluent B: 1 1 acetonitrile+0.5 ml 50% formic acid; gradient: 0.0 min 97% A→0.5 min 97% A→3.2 min 5% A→4.0 min 5% A; oven: 50° C.; flow rate: 0.3 ml/min; UV detection: 210 nm.

Method 3 (LC-MS): Instrument: Waters ACQUITY SQD UPLC System; column: Waters Acquity UPLC HSS T3 1.8μ 30 mm×2 mm; eluent A: 1 1 water+0.25 ml 99% formic acid, eluent B: 1 1 acetonitrile+0.25 ml 99% formic acid; gradient: 0.0 min 90% A→1.2 min 5% A→2.0 min 5% A; oven: 50° C.; flow rate: 0.60 ml/min; UV detection: 208-400 nm.

Method 4 (LC-MS): Instrument: Waters Acquity UPLC-MS SQD 3001; column: Acquity UPLC BEH C18 1.7μ 50 mm×2.1 mm; eluent A: water+0.1% formic acid, eluent B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.

Method 5 (LC-MS): Instrument: Waters Acquity UPLC-MS SQD 3001; column: Acquity UPLC BEH C18 1.7μ 50 mm×2.1 mm; eluent A: water+0.2% ammonia, eluent B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.

Method 6 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile, gradient: A 95%/B 5%→A 55%/B 45%; flow rate: 150 ml/min; UV detection: 254 nm.

Method 7 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile; gradient: A 90%/B 10%→A 50%/B 50%; flow rate: 150 ml/min; UV detection: 254 nm.

Method 8 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile; gradient: A 85%/B 15%→A 45%/B 55%; flow rate: 150 ml/min; UV detection: 254 nm.

Method 9 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile; gradient: A 80%/B 20%→A 40%/B 60%; flow rate: 150 ml/min; UV detection: 254 nm.

Method 10 (HPLC): Instrument: Waters SQD autopurification system; column: Waters XBridge C18 5μ 100 mm×30 mm; eluent A: water+0.1% formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm.

Method 11 (HPLC): Instrument: Waters SQD autopurification system; column: Waters XBridge C18 5μ 100 mm×30 mm; eluent A: water+0.2% ammonia (32%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm.

Method 12 (LC-MS): MS instrument: Waters (Micromass) QM; HPLC instrument: Agilent 1100 series; column: Agilent ZORBAX Extend-C18 3.0 mm×50 mm 3.5 micron; eluent A: 1 1 water+0.01 mol ammonium carbonate, eluent B: 1 1 acetonitrile; gradient: 0.0 min 98% A→0.2 min 98% A→3.0 min 5% A→4.5 min 5% A; oven: 40° C.; flow rate: 1.75 ml/min; UV detection: 210 nm.

Method 13 (LC-MS): Instrument: Waters ACQUITY SQD UPLC System; column: Waters Acquity UPLC HSS T3 1.8μ 50 mm×1 mm; eluent A: 1 1 water+0.25 ml 99% formic acid, eluent B: 1 1 acetonitrile+0.25 ml 99% formic acid; gradient: 0.0 min 95% A→6.0 min 5% A→7.5 min 5% A; oven: 50° C.; flow rate: 0.35 ml/min; UV detection: 210-400 nm.

Method 14 (LC-MS): MS instrument: Waters (Micromass) Quattro Micro; HPLC instrument: Agilent 1100 Series; column: YMC-Triart C18 3μ 50 mm×3 mm; eluent A: 1 1 water+0.01 mol ammonium carbonate, eluent B: 1 1 acetonitrile; gradient: 0.0 min 100% A→2.75 min 5% A→4.5 min 5% A; oven: 40° C.; flow rate: 1.25 ml/min; UV detection: 210 nm.

Method 15 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile; gradient: A 90%/B 10%→A 50%/B 50%; flow rate: 150 ml/min; UV detection: 254 nm.

Method 16 (LC-MS): System: MS Agilent 6110A; HPLC: Agilent 1200 Series; UV DAD; column: Chromolith Flash RP-18e 25-2 mm; eluent A: water with 0.0375% trifluoroacetic acid, eluent B: acetonitrile with 0.0186% trifluoroacetic acid; gradient: 0.0 min 95% A to 0.80 min 5% A to 1.20 min 5% A to 1.21 min 95% A to 1.50 min 95% A; flow rate: 1.5 ml/min; temperature: 50° C.; UV detection: 220 nm and 254 nm.

Method 17 (LC-MS): Instrument: Agilent 1200 Series, 1956AMSD; column: X Bridge C18, 2.1 mm×50 mm, 5 μm; eluent A: 0.05% aqueous ammonia in water, eluent B: acetonitrile; gradient: 0.0 min 95% A to 0.40 min 95% A to 3.40 min 90% A to 4.0 min 0% A to 4.01 min 95% A; flow rate: 0.8 ml/min; temperature: 50° C.; UV detection: 220 nm and 254 nm.

Microwave: The microwave reactor used was an instrument of the Biotage™ Initiator type.

When inventive compounds are purified by preparative HPLC by the above-described methods in which the eluents contain additives, for example trifluoroacetic acid, formic acid or ammonia, the inventive compounds may be obtained in salt form, for example as trifluoroacetate, formate or ammonium salt, if the inventive compounds contain a sufficiently basic or acidic functionality Such a salt can be converted to the corresponding free base or acid by various methods known to the person skilled in the art. Weaker salts can be converted to the corresponding chlorides by addition of a little hydrochloride.

If, in the synthesis intermediates and working examples of the invention described below, a compound is given in the form of a salt of the corresponding base or acid, the exact stoichiometric composition of such a salt as obtained by the respective preparation and/or purification process is generally not known. Unless specified in more detail, additions to names and structural formulae, such as “hydrochloride”, “trifluoroacetate”, “sodium salt” or “x HCl”, “x CF₃COOH”, “x Na⁺” are not to be understood stoichiometrically in the case of such salts, but have only descriptive character with regard to the salt-forming components comprised therein.

This applies correspondingly if the synthesis intermediates and working examples or salts thereof were obtained by the preparation and/or purification processes described in the form of solvates, for example hydrates, whose stoichiometric composition (if of a defined type) is not known.

If the starting compounds and examples contain an L-phenylalanine derivative as the central unit, the corresponding stereocentre is described as the (S) configuration. In the absence of further information, there was no check in individual cases as to whether partial epimerization of the stereocentre took place in the coupling of the L-phenylalanine intermediate with the amine H₂N-R¹. Thus, a mixture of the inventive compounds of (S) enantiomer and (R) enantiomer may be present. The main component is the (S) enantiomer depicted in each case.

Starting Compounds

### Example 1A

### Methyl 4-bromo-N-[(trans-4-{[(tert-butoxycarbonyl)amino]methyl}cyclohexyl)carbonyl]-L-phenylalaninate

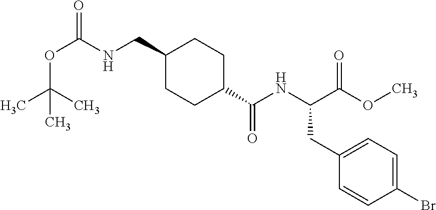

A solution of methyl 4-bromo-L-phenylalaninate (250 g, 874 mmol) and trans-4-{[(tert-butoxycarbonyl)amino]methyl}cyclohexanecarboxylic acid (225 g, 874 mmol) in ethyl acetate (5012 ml) was admixed with N,N-diisopropylethylamine (381 ml, 2186 mmol). The suspension was admixed dropwise with a 2,4,6-tripropyl-1,3,5,2,4,6-trioxatriphosphinane 2,4,6-trioxide solution (50% in DMF, 766 ml, 1312 mmol) and then the mixture was stirred at RT for 3 h. The reaction mixture was stirred into water and extracted three times with ethyl acetate. The organic phase was washed with saturated aqueous sodium hydrogencarbonate solution, saturated aqueous ammonium chloride solution, and saturated aqueous sodium chloride solution. The solution was dried over sodium sulphate and the solvent was removed. This gave 420 g (97% of theory) of the title compound.

¹H NMR (400 MHz, DMSO-d₆): δ=ppm 0.68-0.92 (m, 2H), 1.04-1.32 (m, 4H), 1.37 (s, 9H), 1.48-1.73 (m, 4H), 2.03 (m, 1H), 2.74 (m, 2H), 2.78-2.90 (m, 1H), 2.94-3.05 (m, 1H), 4.36-4.50 (m, 1H), 6.72-6.85 (m, 1H), 7.17 (d, 2H), 7.46 (d, 2H), 8.15 (d, 1H).

LC-MS (Method 1): Rₜ=1.14 min; MS (ESIpos): m/z=497 [M+H]⁺.

### Example 2A

### 4-Bromo-N-[(trans-4-{[(tert-butoxycarbonyl)amino]methyl}cyclohexyl)carbonyl]-L-phenylalanine

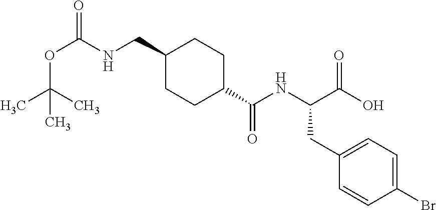

A solution of methyl 4-bromo-N-[(trans-4-{[(tert-butoxycarbonyl)amino]methyl}-cyclohexyl)carbonyl]-L-phenylalaninate in tetrahydrofuran (3000 ml) was admixed with a solution of lithium hydroxide (72 g, 3015 mmol) in water (600 ml). The suspension was stirred at RT for 16 h. The reaction mixture was acidified with 1N hydrochloric acid solution and admixed with ethyl acetate. The organic phase was washed with saturated aqueous sodium chloride solution and dried over sodium sulphate, and the solvent was removed. This gave 284 g (97% of theory) of the title compound.

¹H NMR (400 MHz, DMSO-d₆): δ=ppm 0.71-0.90 (m, 2H), 1.22 (d, 4H), 1.37 (s, 9H), 1.45-1.73 (m, 5H), 2.03 (m, 1H), 2.67-2.88 (m, 3H), 2.95-3.09 (m, 1H), 4.38 (m, 1H), 6.77 (s, 1H), 7.17 (d, 2H), 7.46 (d, 2H), 7.99 (d, 1H), 12.65 (br. s, 1H).

LC-MS (Method 1): Rₜ=1.03 min; MS (ESIneg): m/z=481 [M−H]⁻.

### Example 3A

### 4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide

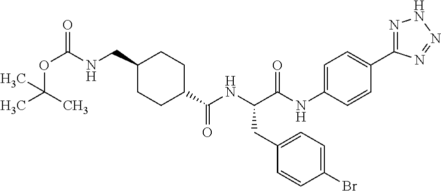

A solution of 4-bromo-N-[(trans-4-[(tert-butoxycarbonyl)amino]methyl}cyclohexyl)carbonyl]-L-phenylalanine (11 g, 22 mmol) and 4-(2H-tetrazol-5-yl)aniline (4 g, 24 mmol) in DMF (161 ml) was admixed with N,N-diisopropylethylamine (9.6 ml, 55 mmol). The suspension was admixed dropwise at 0° C. with a 2,4,6-tripropyl-1,3,5,2,4,6-trioxatriphosphinane 2,4,6-trioxide solution (50% in DMF, 16.9 g, 27 mmol) and then the mixture was stirred at RT for 16 h. The reaction mixture was stirred into ethyl acetate (13000 ml) and extracted three times with water (1570 ml each time). The organic phase was dried with sodium sulphate and the solvent was removed. The crude product was stirred with acetonitrile and filtered off with suction. This gave 11.4 g (78% of theory, 95% purity) of the title compound.

¹H NMR (400 MHz, DMSO-d₆): δ=ppm 0.67-0.90 (m, 2H), 1.24 (m, 4H), 1.37 (s, 9H), 1.51-1.74 (m, 4H), 2.02-2.17 (m, 1H), 2.71-2.79 (m, 2H), 2.79-2.89 (m, 1H), 2.99-3.06 (m, 1H), 3.06-3.16 (m, 1H), 3.51-3.67 (m, 1H), 4.55-4.74 (m, 1H), 6.01-6.02 (m, 1H), 6.69-6.84 (m, 1H), 7.21-7.32 (m, 2H), 7.43-7.55 (m, 2H), 7.64-7.76 (m, 2H), 7.88-7.99 (m, 2H), 8.03-8.14 (m, 1H), 10.25 (s, 1H).

LC-MS (Method 1): Rₜ=1.07 min; MS (ESIneg): m/z=624 [M−H]⁻.

### Example 4A

### 4-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide

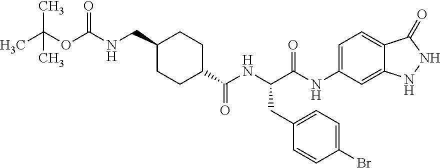

A solution of 4-bromo-N-[(trans-4-{[(tert-butoxycarbonyl)amino]methyl}cyclohexyl)carbonyl]-L-phenylalanine (1500 mg, 3 mmol) and 6-amino-1,2-dihydro-3H-indazol-3-one (555 mg, 24 mmol) in ethyl acetate (21 ml) was admixed with N,N-diisopropylethylamine (1.4 ml, 7.8 mmol). The suspension was admixed with a 2,4,6-tripropyl-1,3,5,2,4,6-trioxatriphosphinane 2,4,6-trioxide solution (50% in DMF, 2.2 ml, 3 7 mmol) and with DMF until dissolution, and then the mixture was stirred at RT for 16 h. The reaction mixture was stirred into ethyl acetate, and washed twice with water and once with saturated aqueous sodium chloride solution. The organic phase was dried with sodium sulphate and the solvent was removed. The crude product was stirred with acetonitrile and filtered off with suction. The residue was separated twice by means of preparative HPLC (eluent: gradient of acetonitrile/water with 0.1% trifluoroacetic acid). The crude product was stirred with methanol and filtered off with suction. This gave 202 mg (11% of theory) of the title compound.

¹H NMR (400 MHz, DMSO-d₆): δ=ppm 0.69-0.89 (m, 2H), 1.04-1.29 (m, 3H), 1.37 (s, 9H), 1.67 (m, 4H), 2.04-2.17 (m, 1H), 2.75 (m, 3H), 2.94-3.07 (m, 1H), 4.54-4.75 (m, 1H), 6.68-6.83 (m, 1H), 6.96 (dd, 1H), 7.25 (d, 2H), 7.39-7.56 (m, 3H), 7.84 (s, 1H), 8.09 (d, 1H), 10.20 (s, 1H), 11.08 (br. s, 1H).

LC-MS (Method 1): Rₜ=1.00 min; MS (ESIpos): m/z=614 [M+H]⁺.

### Example 5A

### 4-Bromo-N-alpha-[(trans-4-{[(tert-butoxycarbonyl)amino]methyl}cyclohexyl)carbonyl]-N-[3-fluoro-4-(2H-tetrazol-5-yl)phenyl]-L-phenylalaninamide

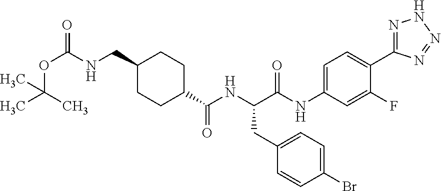

A solution of 4-bromo-N-[(trans-4-{[(tert-butoxycarbonyl)amino]methyl}cyclohexyl)carbonyl]-L-phenylalanine (10 g, 20.7 mmol) and 3-fluoro-4-(2H-tetrazol-5-yl)aniline (4.1 g, 22.8 mmol) in ethyl acetate (210 ml) was admixed with N,N-diisopropylethylamine (10.8 ml, 62.1 mmol). Subsequently, 2,4,6-tripropyl-1,3,5,2,4,6-trioxatriphosphinane 2,4,6-trioxide solution (50% in ethyl acetate, 32.9 g, 52 mmol) was added, and the reaction mixture was refluxed for 2 h and then stirred at RT for 48 h. The reaction mixture was admixed with water and the solid formed was filtered off with suction through a frit, washed with ethyl acetate and dried under reduced pressure. This gave 3.97g (30% of theory) of the title compound.

¹H NMR (300 MHz, DMSO-d6): δ=ppm 0.81 (m, 2H), 1.06-1.29 (m, 3H), 1.36 (s, 9H), 1.46-1.74 (m, 4H), 2.02-2.16 (m, 1H), 2.74 (m, 2H), 2.87 (dd, 1H), 3.00 (dd, 1H), 4.53-4.72 (m, 1H), 6.65-6.79 (m, 1H), 7.24 (d, 2H), 7.39-7.56 (m, 3H), 7.83 (dd, 1H), 8.00 (t, 1H), 8.15 (d, 1H), 10.61 (s, 1H).

LC-MS (Method 4): Rₜ=1.23 min; MS (ESIpos): m/z=645.3 [M+H]⁺.

### Example 6A

### N-alpha-[(trans-4-{[(tert-Butoxycarbonyl)amino]methyl}cyclohexyl)carbonyl]-4-(4,4,5,5-tetramethyl-1,3,2-dioxaborolan-2-yl)-N-[4-(2H-tetrazol-5-yl)phenyl]-L-phenylalaninamide

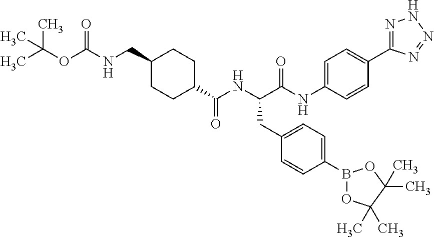

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (4.1 g, 6.5 mmol) and 4,4,4′,4′,5,5,5′,5′-octamethyl-2,2′-bi-1,3,2-dioxaborolane (2.5 g, 9.8 mmol) were dissolved in 41 ml of DMSO, freed of air with argon and blanketed. The reaction mixture was admixed with 1,1′-bis(diphenylphosphino)ferrocene-dichloropalladium(II) (267 mg, 0.16 mmol) and potassium acetate (1.9 g, 19.6 mmol) and stirred in a microwave (Biotage Initiator) at 110° C. for 24 h and at 150° C. for 30 min, and then converted further as the crude product.

LC-MS (Method 4): Rₜ=1.33 min; MS (ESIpos): m/z=674.6 [M+H]⁺.

### Example 7A

### N-alpha-[(trans-4-{[(tert-Butoxycarbonyl)amino]methyl}cyclohexyl)carbonyl]-4-(1H-pyrrolo[2,3-b]pyridin-5-yl)-N-[4-(2H-tetrazol-5-yl)phenyl]-L-phenylalaninamide trifluoroacetate

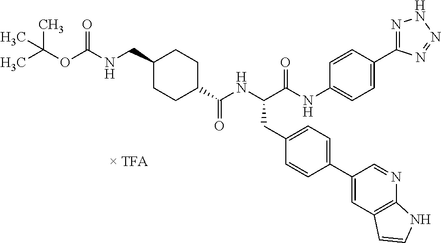

100 mg (0.16 mmol) of 4-bromo-N-alpha-[(trans-4-{[(tert-butoxycarbonyl)amino]methyl}-cyclohexyl)carbonyl]-N-[4-(2H-tetrazol-5-yl)phenyl]-L-phenylalaninamide and 18.4 mg (0.02 mmol) of tetrakis(triphenylphosphine)palladium(0) were taken up in 1.5 ml of 1,2-dimethoxyethane and stirred at RT for 10 min. A solution of 117 mg (0.48 mmol) of 5-(4,4,5,5-tetramethyl-1,3,2-dioxaborolan-2-yl)-1H-pyrrolo[2,3-b]pyridine in 0.50 ml of ethanol was added dropwise to the reaction mixture. After the addition of 1.2 ml of 2N aqueous sodium carbonate solution, the mixture was stirred under reflux for 3 h and at RT for 16 h. The reaction mixture was admixed with IN aqueous hydrochloric acid, the phases were separated and the aqueous phase was extracted three times with ethyl acetate. The organic phases were freed of the solvent on a rotary evaporator, and the residue was separated directly by means of preparative HPLC (eluent: gradient of acetonitrile/water with 0.1% trifluoroacetic acid). This gave 92 mg (85% of theory) of the title compound.

LC-MS (Method 1): Rₜ=0.98 min; MS (ESIpos): m/z=664 [M+H−TFA]⁺.

### Example 8A

### tert-Butyl[(trans-4-{[(2S)-3-[4-(isoquinolin-4-yl)phenyl]-1-oxo-1-{[4-(2H-tetrazol-5-yl)phenyl]amino}propan-2-yl]carbamoyl}cyclohexyl)methyl]carbamate trifluoroacetate

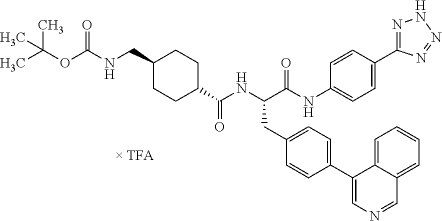

100 mg (0.16 mmol) of 4-bromo-N-alpha-[(trans-4-{[(tert-butoxycarbonyl)amino]methyl}-cyclohexyl)carbonyl]-N-[4-(2H-tetrazol-5-yl)phenyl]-L-phenylalaninamide and 18 mg (0.02 mmol) of tetrakis(triphenylphosphine)palladium(0) were taken up in 1.4 ml of 1,2-dimethoxyethane under argon and stirred at RT for 10 min. A solution of 83 mg (0.48 mmol) of isoquinolin-4-ylboronic acid in 0.54 ml of ethanol was added dropwise to the reaction mixture and stirred at RT for a further 10 min. After the addition of 1.2 ml of 2N aqueous sodium carbonate solution, the mixture was stirred at RT for 5 min and under reflux for 3 h. The reaction mixture was admixed with a little methanol, filtered through a Millipore syringe filter and separated by means of preparative HPLC (eluent: gradient of acetonitrile/water with 0.1% trifluoroacetic acid). This gave 72 mg (52% of theory, 91% purity) of the title compound.

LC-MS (Method 1): Rₜ=0.94 min; MS (ESIneg): m/z=673 [M−H−TFA]⁻.

### Example 9A

### N-alpha-[(trans-4-{[(tert-Butoxycarbonyl)amino]methyl}cyclohexyl)carbonyl]-4-(3-methyl-2-oxo-2,3-dihydro-1H-benzimidazol-5-yl)-N-[4-(2H-tetrazol-5-yl)phenyl]-L-phenylalaninamide

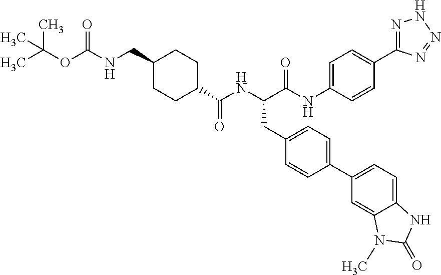

A solution of 150 mg (0.23 mmol) of 4-bromo-N-alpha-[(trans-4-{[(tert-butoxycarbonyl)-amino]methyl}cyclohexyl)carbonyl]-N-[4-(2H-tetrazol-5-yl)phenyl]-L-phenylalaninamide and 98 mg (0.47 mmol) of 1-methyl-6-(4,4,5,5-tetramethyl-1,3,2-dioxaborolan-2-yl)-1,3-dihydro-2H-benzimidazol-2-one in 2.5 ml N,N-dimethylformamide was admixed with 0.36 ml (0.72 mmol) of 2M sodium carbonate solution in water and degassed with argon for 5 min. 35 mg (0.05 mmol) of 1,1′-bis(diphenylphosphino)ferrocenepalladium(II) chloride were added and the mixture was stirred at 120° C. in a preheated oil bath for 30 min. Another 17.5 mg (0.03 mmol) of 1,1′-bis(diphenylphosphino)ferrocenepalladium(II) chloride were added and the mixture was stirred at 120° C. in a preheated oil bath for 30 min. The reaction solution was filtered through kieselguhr, partitioned between water and ethyl acetate and admixed with 10% citric acid solution. The aqueous phase was extracted twice with ethyl acetate and the combined organic phases were dried over sodium sulphate. The solvent was removed, and the residue was suspended in dichloromethane, filtered, washed with acetonitrile and dried under high vacuum. 30 mg (17% of theory) of the title compound were obtained.

¹H NMR (400 MHz, DMSO-d₆): δ=ppm 0.71-0.91 (m, 2H), 1.03-1.28 (m, 3H), 1.36 (s, 9H), 1.68 (m, 3H), 2.00-2.19 (m, 1H), 2.69-2.80 (m, 3H), 2.88-2.98 (m, 1H), 3.03-3.16 (m, 1H), 2.69-2.80 (m, 3H), 4.59-4.80 (m, 1H), 6.67-6.84 (m, 1H), 7.06-7.21 (m, 2H), 7.25-7.43 (m, 3H), 7.54 (d, 2H), 7.82 (d, 2H), 7.99 (d, 2H), 8.08-8.24 (m, 1H), 10.46 (s, 1H), 10.91 (s, 1H), 16.76 (br. s, 1H).

LC-MS (Method 1): Rₜ=0.95 min; MS (ESIneg): m/z=692 [M−H]⁻.

### Example 10A

### N-alpha-[(trans-4-{[(tert-Butoxycarbonyl)amino]methyl}cyclohexyl)carbonyl]-4-(5-methyl-1H-benzimidazol-6-yl)-N-[4-(2H-tetrazol-5-yl)phenyl]-L-phenylalaninamide

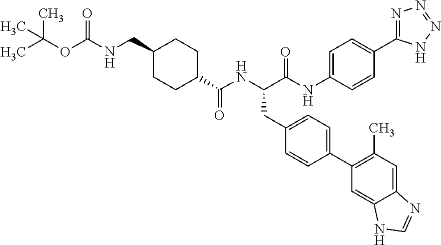

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and tert-butyl 5-methyl-6-(4,4,5,5-tetramethyl -1,3,2-dioxaborolan-2-yl)-1H-benzimidazole-1-carboxylate (128 mg, 0.36 mmol) were dissolved in dimethyl sulphoxide (2 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 120 min, cooled, filtered and purified by chromatography via HPLC (Method 8). This gave 34 mg (21% of theory) of the title compound.

LC-MS (Method 5): Rₜ=0.91 min; MS (ESIpos): m/z=678.5 [M+H−HCl]⁺.

### Example 11A

### N-alpha-[(trans-4-{[(tert-Butoxycarbonyl)amino]methyl}cyclohexyl)carbonyl]-4-(2,4-dimethoxypyrimidin-5-yl)-N-[4-(2H-tetrazol-5-yl)phenyl]-L-phenylalaninamide trifluoroacetate

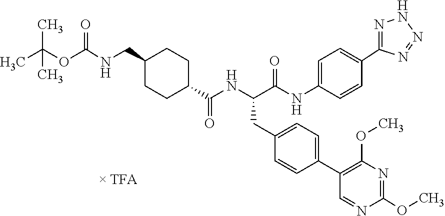 [SMILES: COc1ncc(-c2ccc(C[C@H](NC(=O)[C@H]3CC[C@H](CNC(=O)OC(C)(C)C)CC3)C(=O)Nc3ccc(-c4nn[nH]n4)cc3)cc2)c(OC)n1]

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (250 mg, 0.04 mmol) was dissolved in dimethoxyethane (3.6 ml), degassed and admixed with tetrakis(triphenylphosphine)palladium(0) (46 mg, 40 μmol), 2N aqueous sodium carbonate solution (3 ml) and (2,4-dimethoxypyrimidin-5-yl)boronic acid (220 mg, 1.2 mmol) in ethanol (1.35 ml). The reaction mixture was refluxed for 3 h and cooled. Then (2,4-dimethoxypyrimidin-5-yl)boronic acid (220 mg, 1.2 mmol), tetrakis(triphenylphosphine)palladium(0) (46 mg, 40 μmol), 2N aqueous sodium carbonate solution (1 ml) were added once again and the mixture was refluxed for 3 h. The mixture was filtered and purified by chromatography via HPLC (eluent: gradient of acetonitrile/water with 0.1% trifluoroacetic acid). This gave 160 mg (42% of theory, 85% purity) of the title compound.

LC-MS (Method 1): Rₜ=0.91 min; MS (ESIpos): m/z=686 [M+H−TFA]⁺.

### Example 12A

### N-alpha-[(trans-4-{[(tert-Butoxycarbonyl)amino]methyl}cyclohexyl)carbonyl]-4-(1H-indazol-4-yl)-N-[4-(2H-tetrazol-5-yl)phenyl]-L-phenylalaninamide

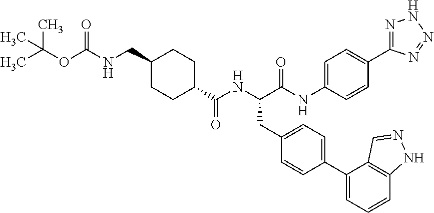 [SMILES: CC(C)(C)OC(=O)NC[C@H]1CC[C@H](C(=O)N[C@@H](Cc2ccc(-c3cccc4[nH]ncc34)cc2)C(=O)Nc2ccc(-c3nn[nH]n3)cc2)CC1]

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.02 mmol) and 1H-indazol-4-ylboronic acid (97 mg, 0.6 mmol) were dissolved in dimethylformamide (2.5 ml), degassed and admixed with 1,1′-bis(diphenylphosphino)ferrocenedichloropalladium(II) (35 mg, 48 μmol) and 2N aqueous sodium carbonate solution (2.5 ml). The reaction mixture was heated at 120° C. for 2 h and cooled. Then 1H-indazol-4-ylboronic acid (32 mg, 0.02 mmol) and 1,1′-bis(diphenylphosphino)ferrocenedichloropalladium(II) (18 mg, 24 μmol) were added once again and the mixture was heated at 120° C. for 1.5 h. The reaction solution was filtered through kieselguhr, partitioned between water and ethyl acetate and admixed with 10% citric acid solution. The aqueous phase was extracted twice with ethyl acetate and the combined organic phases were dried over sodium sulphate. The solvent was removed, and the residue was suspended in ethyl acetate, filtered, washed with acetonitrile and dried under high vacuum. This gave 78 mg (48% of theory) of the title compound.

LC-MS (Method 12): Rₜ=2.02 min; MS (ESIpos): m/z=664 [M+H]⁺.

### Example 13A

### N-alpha-[(trans-4-{[(tert-Butoxycarbonyl)amino]methyl}cyclohexyl)carbonyl]-4-(5-methyl-1H-benzimidazol-6-yl)-N-(3-oxo-2,3-dihydro-1H-indazol-6-yl)-L-phenylalaninamide

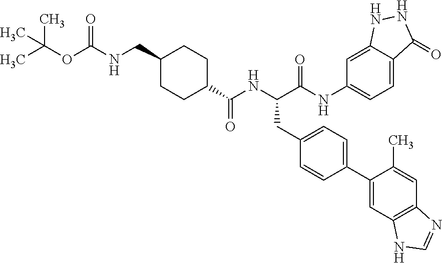

4-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide (150 mg, 0.24 mmol) and tert-butyl 5-methyl-6-(4,4,5,5-tetramethyl-1,3,2-dioxaborolan-2-yl)-1H-benzimidazole-1-carboxylate (135 mg, 0.37 mmol) were dissolved in dimethyl sulphoxide (2 ml) and admixed with tetrakis(triphenylphosphine)palladium(0) (28 mg, 24 μmol), sodium carbonate (77 mg, 0.73 mmol) and water (0.37 ml, 20 mmol). The reaction mixture was stirred at 110° C. in a microwave (Biotage Initiator) for 90 min, cooled, filtered and purified by chromatography via HPLC (Method 10). This gave 23 mg (14% of theory) of the title compound.

LC-MS (Method 4): Rₜ=0.78 min; MS (ESIpos): m/z=665 [M+H]⁺.

### Example 14A

### tert-Butyl[(trans-4-{[(2S)-1-oxo-3-[4-(2-oxo-1,2,3,4-tetrahydroquinolin-7-yl)phenyl]-1-{[4-(2H-tetrazol-5-yl)phenyl]amino}propan-2-yl]carbamoyl}cyclohexyl)methyl]carbamate

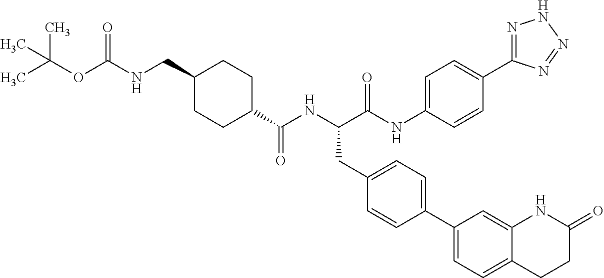

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and (2-oxo-1,2,3,4-tetrahydroquinolin-7-yl)boronic acid (69 mg, 0.36 mmol) were dissolved in dimethyl sulphoxide (1 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 120 min, cooled, filtered and purified by chromatography via HPLC (Method 9). This gave 59 mg (36% of theory) of the title compound.

LC-MS (Method 4): Rₜ=1.11 min; MS (ESIpos): m/z=692.3[M+H]⁺.

### Example 15A

### tert-Butyl[(trans-4-{[(2S)-3-(2′-cyano-3′-fluorobiphenyl-4-yl)-1-oxo-1-{[4-(2H-tetrazol-5-yl)phenyl]amino}propan-2-yl]carbamoyl}cyclohexyl)methyl]carbamate

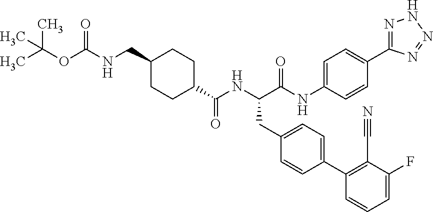

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (300 mg, 0.48 mmol) and 2-fluoro-6-(4,4,5,5-tetramethyl-1,3,2-dioxaborolan-2-yl)benzonitrile (177 mg, 0.72 mmol) were dissolved in dimethyl sulphoxide (3.6 ml) and admixed with tetrakis(triphenylphosphine)palladium(0) (55 mg, 48 μmol), sodium carbonate (152 mg, 0.72 mmol) and water (0.7 ml). The reaction mixture was stirred at 110° C. in a microwave (Biotage Initiator) for 120 min, cooled, filtered and purified by chromatography via HPLC (Method 10). This gave 103 mg (32% of theory) of the title compound.

LC-MS (Method 4): Rₜ=1.26 min; MS (ESIpos): m/z=666.3 [M+H]⁺.

### Example 16A

### 4-(3-Amino-1H-indazol-4-yl)-N-alpha-[(trans-4-{[(tert-butoxycarbonyl)amino]methyl}-cyclohexyl)carbonyl]-N-[4-(2H-tetrazol-5-yl)phenyl]-L-phenylalaninamide

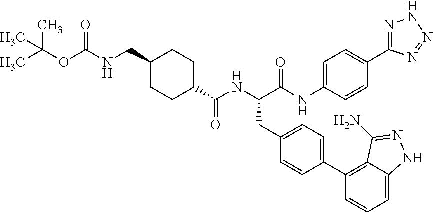

tert-Butyl[(trans-4-{[(25)-3-(2′-cyano-3′-fluorobiphenyl-4-yl)-1-oxo-1-{[4-(2H-tetrazol-5-yl)-phenyl]amino}propan-2-yl]carbamoyl}cyclohexyl)methyl]carbamate (103 mg, 0.15 mmol) and hydrazine (141 mg, 1.5 mmol) were dissolved in ethanol (3.5 ml) and stirred at 100° C. for 3 h. Then hydrazine (86 mg, 0.9 mmol) was added and the mixture was stirred at 100° C. for 4 h. Then hydrazine (283 mg, 3.1 mmol) was added and the mixture was stirred at 110° C. for 16 h. The solvent was removed and the residue was purified by chromatography via HPLC (Method 8). This gave 17 mg (16% of theory) of the title compound.

LC-MS (Method 4): Rₜ=1.07 min; MS (ESIpos): m/z=678.3 [M+H]⁺.

### Example 17A

### tert-Butyl[trans-4-({(2S)-1-{[3-fluoro-4-(2H-tetrazol-5-yl)phenyl]aminol-1-oxo-3-[4-(2-oxo-1,2,3,4-tetrahydroquinolin-7-yl)phenyl]propan-2-yl}carbamoyl)cyclohexyl]methyl}carbamate

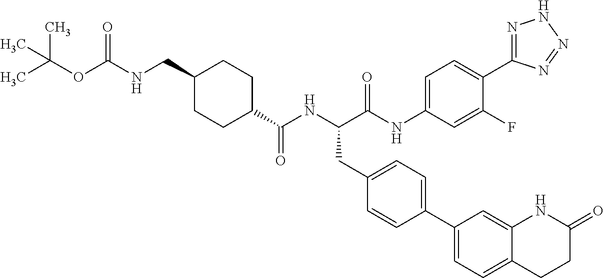

4-Bromo-N-alpha-[(trans-4-{[(tert-butoxycarbonyl)amino]methyl}cyclohexyl)carbonyl]-N-[3-fluoro-4-(2H-tetrazol-5-yl)phenyl]-L-phenylalaninamide (150 mg, 0.24 mmol) and (2-oxo-1,2,3,4-tetrahydroquinolin-7-yl)boronic acid (70 mg, 0.35 mmol) were dissolved in dimethyl sulphoxide (2 ml) and admixed with tetrakis(triphenylphosphine)palladium(0) (27 mg, 24 μmol), sodium carbonate (74 mg, 0.70 mmol) and water (0.35 ml). The reaction mixture was stirred at 110° C. in a microwave (Biotage Initiator) for 90 min, cooled, filtered and purified by chromatography via HPLC (Method 11). This gave 53 mg (32% of theory) of the title compound.

LC-MS (Method 5): Rₜ=0.84 min; MS (ESIpos): m/z=710.3 [M+H]⁺.

### Example 18A

### 4-(1-{2-[(tert-Butoxycarbonyl)amino]ethyl}-1H-benzimidazol-5-yl)-N-alpha-[(trans-4-{[(tert-butoxycarbonyl)amino]methyl}cyclohexyl)carbonyl]-N-[4-(2H-tetrazol-5-yl)phenyl]-L-phenylalaninamide

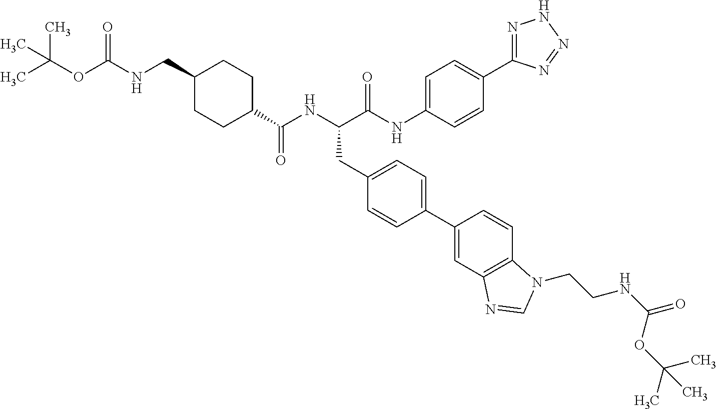

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and tert-butyl{2-[5-(4,4,5,5-tetramethyl-1,3,2-dioxaborolan-2-yl)-1H-benzimidazol-1-yl]ethyl}carbamate (139 mg, 0.36 mmol) were dissolved in dimethyl sulphoxide (1 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 120 min, cooled, filtered and purified by chromatography via HPLC (Method 8). This gave 50 mg (26% of theory) of the title compound.

LC-MS (Method 4): Rₜ=1.04 min; MS (ESIpos): m/z=805.4 [M+H]⁺.

### Example 19A

### N-alpha-[(trans-4-{[(tert-Butoxycarbonyl)amino]methyl}cyclohexyl)carbonyl]-4-(2,4-dioxo-1,2,3,4-tetrahydropyrimidin-5-yl)-N-[4-(2H-tetrazol-5-yl)phenyl]-L-phenylalaninamide

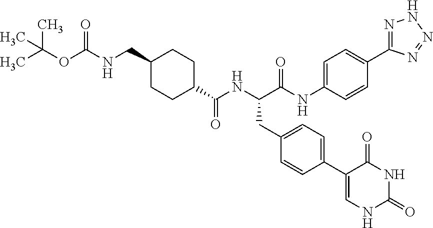

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and (2,4-dioxo-1,2,3,4-tetrahydropyrimidin-5-yl)boronic acid (56 mg, 0.36 mmol) were dissolved in dimethyl sulphoxide (1.8 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 150 min, cooled, filtered and purified by chromatography via HPLC (Method 11). This gave 30 mg (19% of theory) of the title compound.

LC-MS (Method 5): Rₜ=0.60 min; MS (ESIpos): m/z=657.3 [M+H]⁺.

### Example 20A

### N-alpha-[(trans-4-{[(tert-Butoxycarbonyl)amino]methyl}cyclohexyl)carbonyl]-4-(1-methyl-1H-benzimidazol-6-yl)-N-[4-(2H-tetrazol-5-yl)phenyl]-L-phenylalaninamide

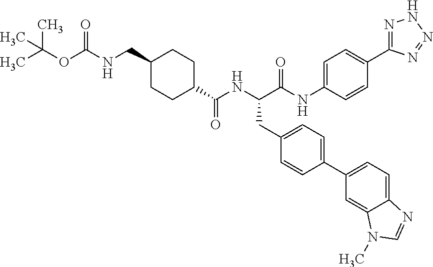

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and (1-methyl-1H-benzimidazol-6-yl)boronic acid (63 mg, 0.36 mmol) were dissolved in dimethyl sulphoxide (1.8 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 90 min, cooled, filtered and purified by chromatography via HPLC (Method 10). This gave 27 mg (17% of theory) of the title compound.

LC-MS (Method 4): Rₜ=0.94 min; MS (ESIpos): m/z=677.3 [M+H]⁺.

### Example 21A

### N-alpha-[(trans-4-{[(tert-Butoxycarbonyl)amino]methyl}cyclohexyl)carbonyl]-4-(1-methyl-1H-benzimidazol-5-yl)-N-[4-(2H-tetrazol-5-yl)phenyl]-L-phenylalaninamide

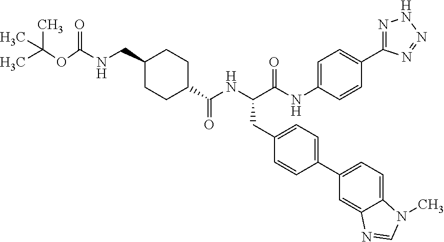

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and (1-methyl-1H-benzimidazol-5-yl)boronic acid (63 mg, 0.36 mmol) were dissolved in dimethyl sulphoxide (1.8 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 90 min, cooled, filtered and purified by chromatography via HPLC (Method 10). This gave 11 mg (7% of theory) of the title compound.

LC-MS (Method 4): Rₜ=0.94 min; MS (ESIpos): m/z=677.3 [M+H]⁺.

### Example 22A

### N-alpha-[(trans-4-{[(tert-Butoxycarbonyl)amino]methyl}cyclohexyl)carbonyl]-4-(2-methyl-1H-benzimidazol-5-yl)-N-[4-(2H-tetrazol-5-yl)phenyl]-L-phenylalaninamide

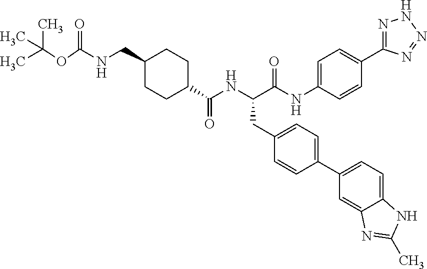

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and (2-methyl-1H-benzimidazol-5-yl)boronic acid (63 mg, 0.36 mmol) were dissolved in dimethyl sulphoxide (1.8 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 90 min, cooled, filtered and purified by chromatography via HPLC (Method 10). This gave 23 mg (14% of theory) of the title compound.

LC-MS (Method 4): Rₜ=0.92 min; MS (ESIpos): m/z=677.3 [M+H]⁺.

### Example 23A

### N-alpha-[(trans-4-{[(tert-Butoxycarbonyl)amino]methyl}cyclohexyl)carbonyl]-4-(5-methyl-1H-indazol-4-yl)-N-[4-(2H-tetrazol-5-yl)phenyl]-L-phenylalaninamide

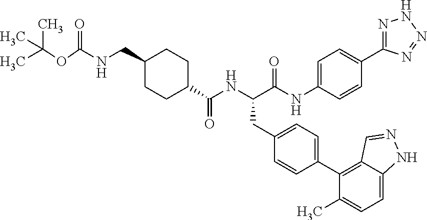

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and (5-methyl-1H-indazol-4-yl)boronic acid (63 mg, 0.36 mmol) were dissolved in dimethyl sulphoxide (1.8 ml) and admixed with tetrakis(triphenylphosphine)palladium(0) (28 mg, 24 μmol), sodium carbonate (76 mg, 0.72 mmol) and water (0.36 ml, 20 mmol). The reaction mixture was stirred at 110° C. in a microwave (Biotage Initiator) for 90 min, cooled, filtered and purified by chromatography via HPLC (Method 10). This gave 20 mg (13% of theory) of the title compound.

LC-MS (Method 4): 1.19 min; MS (ESIpos): m/z=677.3 [M+H]⁺.

### Example 24A

### 4-(1-Benzyl-2,4-dioxo-1,2,3,4-tetrahydropyrimidin-5-yl)-N-alpha-[(trans-4-{[(tert-butoxycarbonyl)amino]methyl}cyclohexyl)carbonyl]-N-[4-(2H-tetrazol-5-yl)phenyl]-L-phenylalaninamide

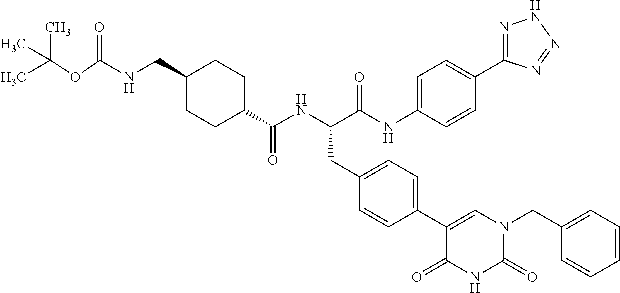

N-alpha-[(trans-4-{[(tert-Butoxycarbonyl)amino]methyl}cyclohexyl)carbonyl]-4-(4,4,5,5-tetramethyl-1,3,2-dioxaborolan-2-yl)-N-[4-(2H-tetrazol-5-yl)phenyl]-L-phenylalaninamide (200 mg, 0.30 mmol) and 1-benzyl-5-bromopyrimidine-2,4(1H,3H)-dione (100 mg, 0.36 mmol) were dissolved in dimethyl sulphoxide (1.8 ml) and admixed with tetrakis(triphenylphosphine)palladium(0) (34 mg, 30 μmol), sodium carbonate (157 mg, 1.48 mmol) and water (0.45 ml, 25 mmol). The reaction mixture was stirred at 110° C. in a microwave (Biotage Initiator) for 150 min. After addition of 1,1′-bis(diphenylphosphino)ferrocenedichloropalladium(II), the mixture was stirred at 110° C. in a microwave (Biotage Initiator) for 60 min, cooled, filtered and purified by chromatography via HPLC (Method 11). This gave 25 mg (11% of theory) of the title compound.

LC-MS (Method 5): 0.83 min; MS (ESIpos): m/z=747.4 [M+H]⁺.

### Example 25A

### N-alpha-[(trans-4-{[(tert-Butoxycarbonyl)amino]methyl}cyclohexyl)carbonyl]-4-(1,3-dimethyl-2,4-dioxo-1,2,3,4-tetrahydropyrimidin-5-yl)-N-[4-(2H-tetrazol-5-yl)phenyl]-L-phenylalaninamide

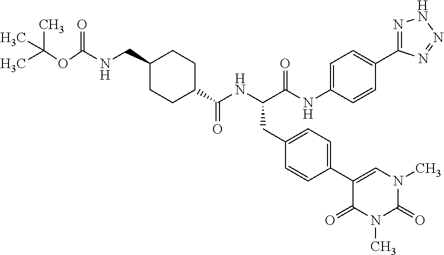

N-alpha-[(trans-4-{[(tert-Butoxycarbonyl)amino]methyl}cyclohexyl)carbonyl]-4-(4,4,5,5-tetramethyl-1,3,2-dioxaborolan-2-yl)-N-[4-(2H-tetrazol-5-yl)phenyl]-L-phenylalaninamide (200 mg, 0.30 mmol) and 5-bromo-1,3-dimethylpyrimidine-2,4(1H,3H)-dione (78 mg, 0.36 mmol) were dissolved in dimethyl sulphoxide (1.8 ml) and admixed with tetrakis(triphenylphosphine)palladium(0) (34 mg, 30 μmol), sodium carbonate (157 mg, 1.48 mmol) and water (0.45 ml, 25 mmol). The reaction mixture was stirred at 110° C. in a microwave (Biotage Initiator) for 150 min. The reaction mixture was stirred at 110° C. in a microwave (Biotage Initiator) for 90 min, cooled, filtered and purified by chromatography via HPLC (Method 11). This gave 14 mg (7% of theory) of the title compound.

LC-MS (Method 5): 0.74 min; MS (ESIpos): m/z=685.3 [M+H]⁺.

### Example 26A

### 4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide

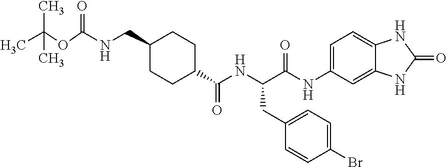

A solution of 4-bromo-N-[(trans-4-{[(tert-butoxycarbonypamino]methyl}cyclohexyl)carbonyl]-L-phenylalanine (5000 mg, 10 mmol) and 5-amino-1,3-dihydro-2H-benzimidazol-2-one (1851 mg, 12 mmol) in ethyl acetate (70 ml) was admixed with N,N-diisopropylethylamine (4.5 ml, 26 mmol). The suspension was admixed with a 2,4,6-tripropyl-1,3,5,2,4,6-trioxatriphosphinane 2,4,6-trioxide solution (50% in dimethylformamide, 7898 mg, 12 mmol) and with dimethylformamide (20 ml) until dissolution, and then the mixture was stirred at RT for 16 h. The reaction mixture was stirred into ethyl acetate (600 ml), and washed three times with water (300 ml) and once with saturated aqueous sodium chloride solution (250 ml). The precipitate in the organic phase was filtered off and washed with ethyl acetate. The solvent of the filtrate was removed and the residue was dried under high vacuum. This gave 4021 mg (62% of theory) of the title compound.

¹H NMR (400 MHz, DMSO-d₆): δ ppm=0.68-0.89 (m, 2H), 1.17 (m, 3H), 1.37 (s, 9H), 1.66 (m, 4H), 2.02-2.15 (m, 1H), 2.74 (m, 3H), 2.93-3.07 (m, 1H), 3.98-4.09 (dd, 1H), 4.52-4.66 (dd, 1H), 6.72-6.88 (m, 2H), 7.02 (dd, 1H), 7.25 (d, 2H), 7.38-7.53 (m, 3H), 8.10 (d, 1H), 10.04 (s, 1H), 10.51 (s, 1H), 10.59 (s, 1H).

LC-MS (Method 1): Rₜ=1.00 min; MS (ESIneg): m/z=612 [M−H]⁻.

### Example 27A

### N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenylalaninamide

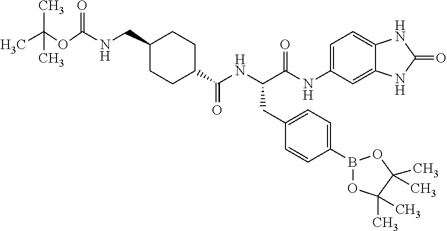

4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (5.0 g, 8.14 mmol) and 4,4,4′,4′,5,5,5′,5′-octamethyl-2,2′-bi-1,3,2-dioxaborolane (3.1 g, 12.2 mmol) were dissolved in 60 ml of DMSO, 1,1′-bis(diphenylphosphino)ferrocene-dichloropalladium(II) (332 mg, 0.4 mmol) and potassium acetate (2.4 g, 24.4 mmol) were added and the mixture was stirred at 110° C. for 4 h and then converted further as crude product.

LC-MS (Method 4): Rₜ=1.27 min; MS (ESIpos): m/z=662.5 [M+H]⁺.

### Example 28A

### 5-Bromo-2-isopropyl-6-methyl-1H-benzimidazole

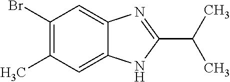

4-Bromo-5-methylbenzene-1,2-diamine (5.0 g, 23.6 mmol) and isobutyraldehyde (2.13, 28.4 mmol) were dissolved in 75 ml of ethanol and admixed with cobalt hydroxide (220 mg, 2.4 mmol). The reaction mixture was stirred uncovered overnight, filtered and concentrated to dryness under reduced pressure. The residue was purified by chromatography via silica gel (Biotage Isolera, SNAP 340 g, eluent: hexane/ethyl acetate 7/3). This gave 1.8 g (30% of theory) of the title compound.

LC-MS (Method 5): Rₜ=1.05 min; MS (ESIpos): m/z=255.0 [M+H]⁺.

### Example 29A

### 2-Isopropyl-6-methyl-5-(4,4,5,5-tetramethyl-1,3,2-dioxaborolan-2-yl)-1H-benzimidazole

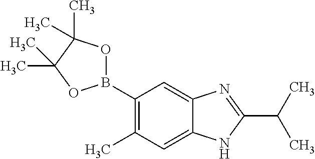 [SMILES: Cc1cc2[nH]c(C(C)C)nc2cc1B1OC(C)(C)C(C)(C)O1]

5-Bromo-2-isopropyl-6-methyl-1H-benzimidazole (120 mg, 0.47 mmol) and 4,4,4′,4′,5,5,5′,5′-octamethyl-2,2′-bi-1,3,2-dioxaborolane (181 mg, 0.71 mmol) were dissolved in 2 ml of DMSO, 1,1′-bis(diphenylphosphino)ferrocene-dichloropalladium(II) (19 mg, 0.024 mmol) and potassium acetate (140 mg, 1.42 mmol) were added and the mixture was stirred at 110° C. for 2 h and then converted further as crude product.

LC-MS (Method 4): Rₜ=1.01 min; MS (ESIpos): m/z=301.2 [M+H]⁺.

### Example 30A

### N-alpha-[(trans-4-{[(tert-Butoxycarbonyl)amino]methyl}cyclohexyl)carbonyl]-4-(2-isopropyl-6-methyl-1H-benzimidazol-5-yl)-N-(2-oxo-2,3-dihydro-1H-benzimidazol-5-yl)-L-phenylalaninamide

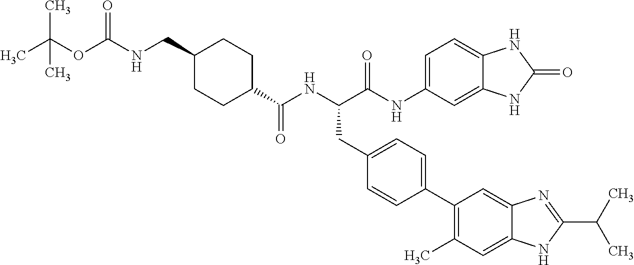

N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenyl-alaninamide (2.0 g, 3.0 mmol) and 5-bromo-2-isopropyl-6-methyl-1H-benzimidazole (842 mg, 3.33 mmol) were dissolved in dimethyl sulphoxide (18 ml) and admixed with 1,1′-bis(diphenylphosphino)ferrocene-dichloropalladium(II) (123 mg, 150 μmol), sodium carbonate (961 mg, 9.1 mmol) and water (4.6 ml, 252 mmol). The reaction mixture was stirred at 120° C. for 2 h. The reaction mixture was admixed with water, and the residue was filtered off, dried and purified by chromatography via silica gel (Biotage Isolera, SNAP NH 375 g, eluent: hexane/ethyl acetate/methanol). This gave 391 mg (18% of theory) of the title compound.

LC-MS (Method 4): 0.92 min; MS (ESIpos): m/z=708.5 [M+H]⁺.

### Example 31A

### 6-Bromo-2-cyclopropyl-5-methyl-1H-benzimidazole

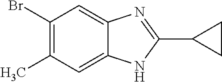

4-Bromo-5-methylbenzene-1,2-diamine (0.5 g, 2.3 mmol) and cyclopropanecarboxaldehyde (207 mg, 2.8 mmol) were dissolved in 7.5 ml of ethanol and admixed with cobalt hydroxide (22 mg, 0.24 mmol). The reaction mixture was stirred uncovered overnight, filtered and concentrated to dryness under reduced pressure. The residue was purified by chromatography via HPLC (2× Labomatic HD-3000 pump, Labomatic AS-3000, Knauer DAD 2600, Labomatic Labcol Vario 4000 Plus Chromatorex C18 10 μm 125 mm×30 mm+250 mm×50.8 mm; eluent A: water+0.2% by vol. of ammonia (32%), eluent B: methanol, gradient: 0-12 min 70-100% B; flow rate: 100 ml/min) This gave 184 mg (31% of theory) of the title compound.

LC-MS (Method 5): Rₜ=1.05 min; MS (ESIpos): m/z=253.0 [M+H]⁺.

### Example 32A

### N-[(trans-4-{[(tert-Butoxycarbonyl)amino]methyl}cyclohexyl)carbonyl]-4-(4,4,5,5-tetramethyl-1,3,2-dioxaborolan-2-yl)-L-phenylalanine

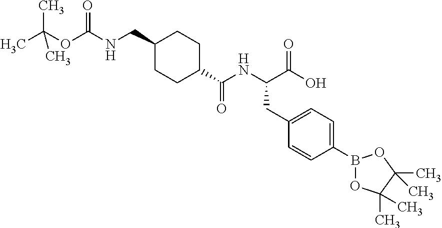

4-Bromo-N-[(trans-4-{[(tert-butoxycarbonyl)amino]methyl}cyclohexyl)carbonyl]-L-phenylalanine (3.6 g, 7.4 mmol) and 4,4,4′,4′,5,5,5′,5′-octamethyl-2,2′-bi-1,3,2-dioxaborolane (2.8 g, 11.2 mmol) were dissolved in 50 ml of DMSO, 1,1′-bis(diphenylphosphino)ferrocene-dichloropalladium(II) (304 mg, 0.37 mmol) and potassium acetate (2.2 g, 22.3 mmol) were added and the mixture was stirred at 110° C. for 5 h and then converted further as crude product.

LC-MS (Method 4): Rₜ=1.32 min; MS (ESIpos): m/z=531.4 [M+H]⁺.

### Example 33A

### N-[(trans-4-{[(tert-Butoxycarbonyl)amino]methyl}cyclohexyl)carbonyl]-4-(2-cyclopropyl-5-methyl-1H-benzimidazol-6-yl)-L-phenylalanine

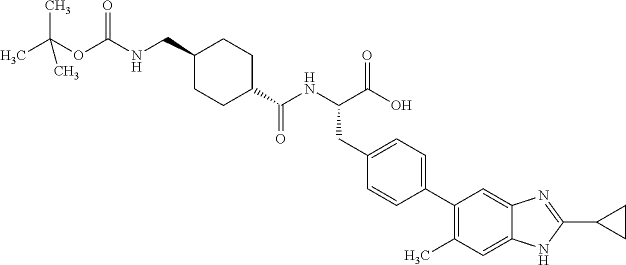

N-[(trans-4-{[(tert-Butoxycarbonyl)amino]methyl}cyclohexyl)carbonyl]-4-(4,4,5,5-tetramethyl-1,3,2-dioxaborolan-2-yl)-L-phenylalanine (2.17 g, 4.1 mmol) and 6-bromo-2-cyclopropyl-5-methyl-1H-benzimidazole (1.13 g, 4.5 mmol) were dissolved in dimethyl sulphoxide (30 ml) and admixed with 1,1′-bis(diphenylphosphino)ferrocene-dichloropalladium(II) (334 mg, 409 μmol), sodium carbonate (1.3 g, 12.3 mmol) and water (6.2 ml, 341 mmol). The reaction mixture was stirred at 120° C. for 2 h. The reaction mixture was filtered through alumina and purified by chromatography via HPLC (Instrument: Labomatic HD3000, AS-3000, Labcol Vario 4000 Plus, Knauer DAD 2600; column: Waters XBridge C18 5μ 150 mm×50 mm; eluent A: water+0.2% by vol. of ammonia (32%), eluent B: acetonitrile; gradient: 0.00-1.00 min 10% B (flow rate: 50 to >150 ml/min), 1.00-8.00 min 10-40% B (flow rate: 150 ml/min), temperature: RT; UV detection: 254 nm). This gave 665 mg (28% of theory) of the title compound.

LC-MS (Method 4): 0.88 min; MS (ESIpos): m/z=575.4 [M+H]⁺.

### Example 34A

### N-alpha-[(trans-4-{[(tert-Butoxycarbonyl)amino]methyl}cyclohexyl)carbonyl]-N-(7-chloro-2-oxo-2,3-dihydro-1H-benzimidazol-5-yl)-4-(2-cyclopropyl-6-methyl-1H-benzimidazol-5-yl)-L-phenylalaninamide

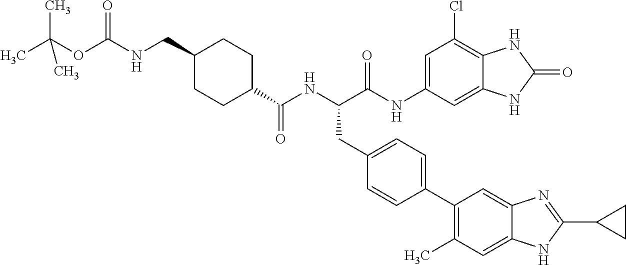

A solution of N-[(trans-4-{[(tert-butoxycarbonyl)amino]methyl}cyclohexyl)carbonyl]-4-(2-cyclopropyl-5-methyl-1H-benzimidazol-6-yl)-L-phenylalanine (150 mg, 0.26 mmol) and 6-amino-4-chloro-1,3-dihydro-2H-benzimidazol-2-one (53 mg, 0.29 mmol) in ethyl acetate (3 ml) was admixed with N,N-diisopropylethylamine (0.1 ml, 0.8 mmol). The suspension was admixed with a 2,4,6-tripropyl-1,3,5,2,4,6-trioxatriphosphinane 2,4,6-trioxide solution (50% in ethyl acetate, 0.5 g, 0.8 mmol) and then stirred under reflux for 3 h. The reaction mixture was admixed with water, and the precipitate was filtered off with suction, dried under high vacuum and purified by chromatography via HPLC (Method 8). This gave 29 mg (15% of theory) of the title compound.

LC-MS (Method 4): 0.92 min; MS (ESIpos): m/z=740.5 [M+H]⁺.

### Example 35A

### 6-Bromo-2-cyclopropyl-5-methyl-3H-imidazo[4,5-b]pyridine

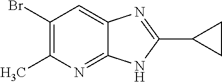

2,3-Diamino-5-bromo-6-methylpyridine (250 mg, 12 mmol) and cyclopropanecarboxaldehyde (104 mg, 1.45 mmol) were dissolved in 4 ml of ethanol and admixed with cobalt hydroxide (12 mg, 0.12 mmol). The reaction mixture was concentrated to dryness under reduced pressure. The residue was purified by chromatography via Biotage Isolera (SNAP 25, hexane/ethyl acetate gradient). This gave 207 mg (66% of theory) of the title compound.

LC-MS (Method 5): Rₜ=0.85 min; MS (ESIpos): m/z=254.0 [M+H]⁺.

### Example 36A

### N-alpha-[(trans-4-{[(tert-Butoxycarbonyl)amino]methyl}cyclohexyl)carbonyl]-4-(2-cyclopropyl-5-methyl-1H-imidazo[4,5-b]pyridin-6-yl)-N-(2-oxo-2,3-dihydro-1H-benzimidazol-5-yl)-L-phenylalaninamide

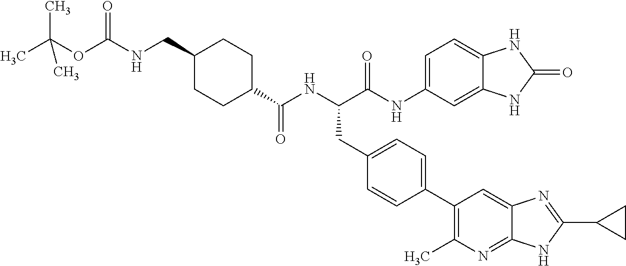

N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenyl-alaninamide (484 mg, 0.73 mmol) and 6-bromo-2-cyclopropyl-5-methyl-3H-imidazo[4,5-b]pyridine (203 mg, 0.8 mmol) were dissolved in dimethyl sulphoxide (4.5 ml) and admixed with 1,1′-bis(diphenylphosphino)ferrocene-dichloropalladium(II) (30 mg, 36 μmol), sodium carbonate (233 mg, 2.6 mmol) and water (1.1 ml, 61 mmol). The reaction mixture was stirred at 120° C. for 2 h. The reaction mixture was admixed with water, and the residue was filtered off, suspended in tetrahydrofuran, filtered off with suction, dried and converted further as the crude product. This gave 471 mg (91% of theory) of the title compound.

LC-MS (Method 4): 0.93 min; MS (ESIpos): m/z=707.5 [M+H]⁺.

### Example 37A

### N-alpha-[(trans-4-{[(tert-Butoxycarbonyl)amino]methyl}cyclohexyl)carbonyl]-4-(2-cyclopropyl-6-methyl-1H-benzimidazol-5-yl)-N-(4-fluoro-3-oxo-2,3-dihydro-1H-indazol-6-yl)-L-phenylalaninamide

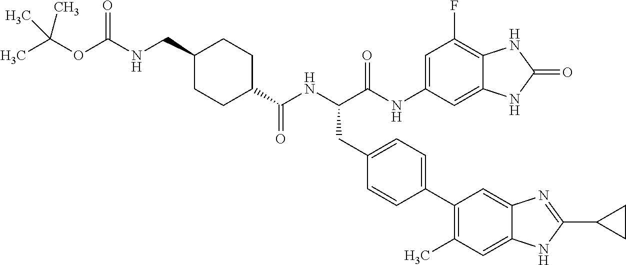

A solution of N-[(trans-4-{[(tert-butoxycarbonyl)amino]methyl}cyclohexyl)carbonyl]-4-(2-cyclopropyl-5-methyl-1H-benzimidazol-6-yl)-L-phenylalanine (150 mg, 0.26 mmol) and 6-amino-4-fluoro-1,2-dihydro-3H-indazol-3-one (48 mg, 0.29 mmol) in ethyl acetate (3 ml) was admixed with N,N-diisopropylethylamine (0.1 ml, 0.8 mmol). The suspension was admixed with a 2,4,6-tripropyl-1,3,5,2,4,6-trioxatriphosphinane 2,4,6-trioxide solution (50% in ethyl acetate, 0.5 g, 0.8 mmol) and then stirred under reflux for 5 h. The reaction mixture was admixed with water, and the precipitate was filtered off with suction, dried under high vacuum and purified by chromatography via HPLC (Method 8). This gave 35 mg (18% of theory) of the title compound.

LC-MS (Method 4): 0.90 min; MS (ESIpos): m/z=724.5 [M+H]⁺.

### Example 38A

### 6-Bromo-5-chloro-2-cyclopropyl-1H-benzimidazole

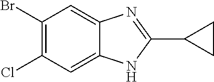 [SMILES: Clc1cc2[nH]c(C3CC3)nc2cc1Br]

4-Bromo-5-chlorobenzene-1,2-diamine (0.5 g, 2.15 mmol) and cyclopropanecarboxaldehyde (184 mg, 2.6 mmol) were dissolved in 7 ml of ethanol and admixed with cobalt hydroxide (22 mg, 0.24 mmol). The reaction mixture was stirred uncovered overnight, filtered and concentrated to dryness under reduced pressure. The residue was purified by chromatography via Biotage Isolera (SNAP 50, hexane/ethyl acetate gradient). This gave 582 mg (15% of theory) of the title compound.

LC-MS (Method 5): Rₜ=1.07 min; MS (ESIpos): m/z=273.0 [M+H]⁺.

### Example 39A

### N-alpha-[(trans-4-{[(tert-Butoxycarbonyl)amino]methyl}cyclohexyl)carbonyl]-4-(6-chloro-2-cyclopropyl-1H-benzimidazol-5-yl)-N-(2-oxo-2,3-dihydro-1H-benzimidazol-5-yl)-L-phenylalaninamide

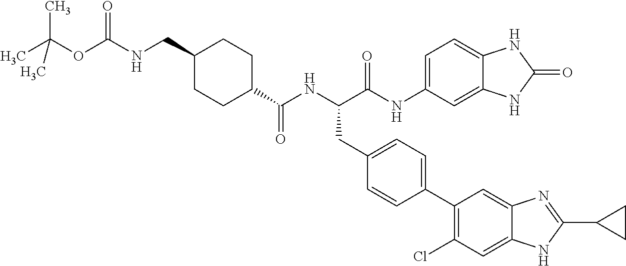

N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenyl-alaninamide (170 mg, 0.26 mmol) and 6-bromo-5-chloro-2-cyclopropyl-1H-benzimidazole (76.5 mg, 0.28 mmol) were dissolved in dimethyl sulphoxide (2 ml) and admixed with 1,1′-bis(diphenylphosphino)ferrocene-dichloropalladium(II) (21 mg, 26 μmol), sodium carbonate (82 mg, 0.77 mmol) and water (0.39 ml, 21.5 mmol). The reaction mixture was stirred at 120° C. for 4 h. The reaction mixture was admixed with water, and the residue was filtered off, dried under reduced pressure and converted further as the crude product. This gave 120 mg (64% of theory) of the title compound.

LC-MS (Method 5): 1.11 min; MS (ESIpos): m/z=726.4 [M+H]⁺.

### Example 40A

### 2,2,3,3-Tetrafluoro-3-[5-(4-nitrophenyl)-1H-1,2,4-triazol-3-yl]propanoic acid

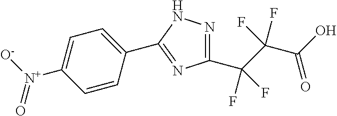 [SMILES: O=C(O)C(F)(F)C(F)(F)c1n[nH]c(-c2ccc([N+](=O)[O-])cc2)n1]

4-Nitrobenzenecarboximidohydrazide (1.22 g, 6.8 mmol) was dissolved in 50 ml of dichloromethane and admixed with 3,3,4,4-tetrafluorodihydrofuran-2,5-dione (3.5 g, 20.3 mmol). The reaction mixture was stirred at RT for 2 min, admixed with 50 ml of acetonitrile and stirred at RT overnight. The reaction mixture was concentrated and converted further as the crude product.

LC-MS (Method 4): Rₜ=0.72 min; MS (ESIneg): m/z=333.1 [M−H]⁻.

### Example 41A

### 3-[5-(4-Aminophenyl)-1H-1,2,4-triazol-3-yl]-2,2,3,3-tetrafluoropropanoic acid

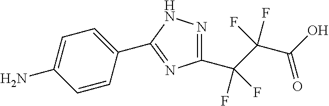 [SMILES: Nc1ccc(-c2nc(C(F)(F)C(F)(F)C(=O)O)n[nH]2)cc1]

2,2,3,3-Tetrafluoro-3-[5-(4-nitrophenyl)-1H-1,2,4-triazol-3-yl]propanoic acid (2.3 g, 69 mmol) was dissolved in 115 ml of methanol, admixed with ammonium formate (1.74 g, 27 5 mmol) and palladium/charcoal (10%, 732 mg, 0.7 mmol) and stirred at RT for 30 min. The reaction mixture was filtered and concentrated and converted further as the crude product.

LC-MS (Method 4): Rₜ=0.45 min; MS (ESIpos): m/z=305.0 [M+H]⁺.

### Example 42A

### 3-{5-[4-({4-Bromo-N-[(trans-4-{[(tert-butoxycarbonyl)amino]methyl}cyclohexyl)carbonyl]-L-phenylalanyl}amino)phenyl]-1H-1,2,4-triazol-3-yl}-2,2,3,3-tetrafluoropropanoic acid

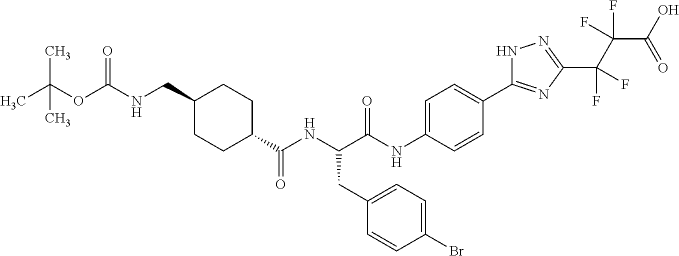

A solution of 4-bromo-N-[(trans-4-{[(tert-butoxycarbonyl)amino]methyl}cyclohexyl)carbonyl]-L-phenylalanine (1 g, 2.1 mmol) and 3-[5-(4-aminophenyl)-1H-1,2,4-triazol-3-yl]-2,2,3,3-tetrafluoropropanoic acid (1.38 g, 23 mmol, 50% purity) in ethyl acetate (125 ml) was admixed with N,N-diisopropylethylamine (1.1 ml, 6.2 mmol). Subsequently, 2,4,6-tripropyl-1,3,5,2,4,6-trioxatriphosphinane 2,4,6-trioxide solution (50% in ethyl acetate, 3.66 ml, 6.2 mmol) was added and the mixture was refluxed for 3 h. The reaction mixture was admixed with water, the phases were separated and the organic phase was washed with saturated aqueous sodium chloride solution, dried over sodium sulphate, filtered and concentrated. This gave 1.74 g (quant.) of the title compound.

LC-MS (Method 5): Rₜ=0.71 min; MS (ESIneg): m/z=767 [M−H]⁻.

### Example 43A

### 3-{5-[4-({N-[(trans-4-{[(tert-Butoxycarbonyl)amino]methyl}cyclohexyl)carbonyl]-4-(2-isopropyl-6-methyl-1H-benzimidazol-5-yl)-L-phenylalanyl}amino)phenyl]-4H-1,2,4-triazol-3-yl}-2,2,3,3-tetrafluoropropanoic acid

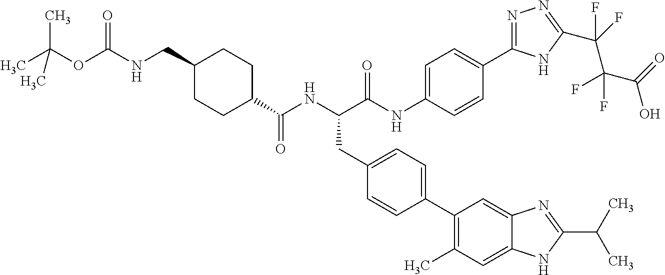

3-{5-[4-({4-Bromo-N-[(trans-4-{[(tert-butoxycarbonyl)amino]methyl}cyclohexyl)carbonyl]-L-phenylalanyl}amino)phenyl]-1H-1,2,4-triazol-3-yl}-2,2,3,3-tetrafluoropropanoic acid (150 mg, 0.2 mmol) and 2-isopropyl-6-methyl-5-(4,4,5,5-tetramethyl-1,3,2-dioxaborolan-2-yl) -1H-benzimidazole (64 mg, 0.2 mmol) were dissolved in dimethyl sulphoxide (2.5 ml) and admixed with 1,1′-bis(diphenylphosphino)ferrocene-dichloropalladium(II) (16 mg, 19.5 μmol), sodium carbonate (62 mg, 0.6 mmol) and water (0.3 ml, 16 mmol). The reaction mixture was stirred at 110° C. for 4 h. The reaction mixture was filtered through deactivated alumina and purified by chromatography via HPLC (Method 7). This gave 16 mg (9% of theory) of the title compound.

LC-MS (Method 5): Rₜ=0.98 min; MS (ESIpos): m/z=863.6 [M+H]⁺.

### Example 44A

### 4-Bromo-N-alpha-[(trans-4-{[(tert-butoxycarbonyl)amino]methyl}cyclohexyl)carbonyl]-N-[2-(pentafluoroethyl)-1H-benzimidazol-6-yl]-L-phenylalaninamide

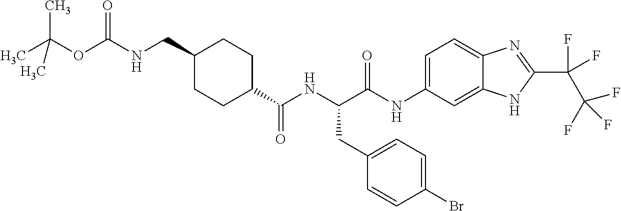

A solution of 4-bromo-N-[(trans-4-{[(tert-butoxycarbonyl)amino]methyl}cyclohexyl)carbonyl]-L-phenylalanine (4.4 g, 9.0 mmol) and 2-(pentafluoroethyl)-1H-benzimidazol-6-amine (2.5 g, 10 mmol) in ethyl acetate (120 ml) was admixed with N,N-diisopropylethylamine (3.8 ml, 27.1 mmol). Subsequently, 2,4,6-tripropyl-1,3,5,2,4,6-trioxatriphosphinane 2,4,6-trioxide solution (50% in ethyl acetate, 16 ml, 27.1 mmol) was added and the mixture was refluxed for 4 h. The reaction mixture was admixed with water, the phases were separated and the organic phase was washed with saturated aqueous sodium chloride solution, dried over sodium sulphate, filtered and concentrated. This gave 5.1 g (78% of theory) of the title compound.

LC-MS (Method 4): Rₜ=1.37 min; MS (ESIpos): m/z=718.2 [M+H]⁺.

### Example 45A

### N-alpha-[(trans-4-{[(tert-Butoxycarbonyl)amino]methyl}cyclohexyl)carbonyl]-4-(2-isopropyl-6-methyl-1H-benzimidazol-5-yl)-N-[2-(pentafluoroethyl)-1H-benzimidazol-6-yl]-L-phenylalaninamide

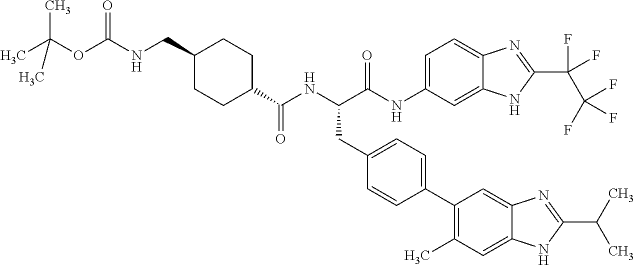

4-Bromo-N-alpha-[(trans-4-{[(tert-butoxycarbonyl)amino]methyl}cyclohexyl)carbonyl]-N-[2-(pentafluoroethyl)-1H-benzimidazol-6-yl]-L-phenylalaninamide (150 mg, 0.2 mmol) and 2-isopropyl-6-methyl-5-(4,4,5,5-tetramethyl-1,3,2-dioxaborolan-2-yl)-1H-benzimidazole (69 mg, 0.23 mmol) were dissolved in dimethyl sulphoxide (2.7 ml) and admixed with 1,1′-bis(diphenylphosphino)ferrocene-dichloropalladium(II) (17 mg, 21 μmol), sodium carbonate (66 mg, 0.63 mmol) and water (0.32 ml, 17.5 mmol). The reaction mixture was stirred at 110° C. for 4 h. The reaction mixture was filtered through deactivated alumina and purified by chromatography via HPLC (Method 9). This gave 18 mg (11% of theory) of the title compound.

LC-MS (Method 5): Rₜ=0.99 min; MS (ESIpos): m/z=810.5 [M+H]⁺.

### Example 46A

### N-alpha-[(trans-4-{[(tert-Butoxycarbonyl)amino]methyl}cyclohexyl)carbonyl]-4-(2-cyclopropyl-6-methyl-1,3-benzoxazol-5-yl)-N-[4-(1H-tetrazol-5-yl)phenyl]-L-phenylalaninamide

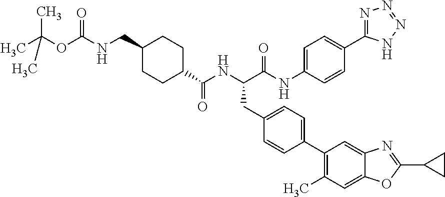

4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (150 mg, 0.24 mmol) and 2-cyclopropyl-6-methyl-5-(4,4,5,5-tetramethyl-1,3,2-dioxaborolan-2-yl)-1,3-benzoxazole (79 mg, 0.26 mmol) were dissolved in dimethyl sulphoxide (3.0 ml) and admixed with 1,1′-bis(diphenylphosphino)ferrocene-dichloropalladium(II) (19 mg, 24 μmol), sodium carbonate (76 mg, 0.72 mmol) and water (0.36 ml, 20 mmol). The reaction mixture was stirred at 110° C. for 4 h. The reaction mixture was filtered through deactivated alumina and purified by chromatography via HPLC (Method 8). This gave 74 mg (43% of theory) of the title compound.

LC-MS (Method 4): Rt=1.34 min; MS (ESIpos): m/z=719.5 [M+H]⁺.

### Example 47A

### 3-Fluoro-4-methylbenzene-1,2-diamine

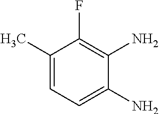

2-Fluoro-3-methyl-6-nitroaniline (4.50 g, 26.45 mmol) was dissolved in ethanol (50 ml), admixed with palladium/charcoal (1.0 g, 10%) and stirred under a hydrogen atmosphere (50 psi) at RT for 24 h. The reaction mixture was filtered via Celite and concentrated under reduced pressure. This gave 3.63 g (98% of theory) of the title compound. This was converted further as the crude product.

LC-MS (Method 16): Rₜ=0.097 min; m/z=141.1 (M+H)⁺.

### Example 48A

### 2-Cyclobutyl-4-fluoro-5-methyl-1H-benzimidazole

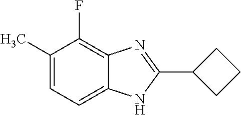

3-Fluoro-4-methylbenzene-1,2-diamine (1.81 g, 12.9 mmol) and cyclobutanecarbaldehyde (1.09 g, 12.9 mmol) were dissolved in ethanol (20 ml) and admixed with cobalt hydroxide (120 mg, 1.29 mmol). The mixture was stirred uncovered at RT for 18 h, filtered and concentrated under reduced pressure. The residue was purified by chromatography via silica gel (hexane/ethyl acetate gradient 10:1 to 3:1). This gave 1.2 g of the title compound as a crude product, which was converted without further purification.

LC-MS (Method 16): Rₜ=0.121 min; m/z=205.1 (M+H)⁺.

### Example 49A

### 6-Bromo-2-cyclobutyl-4-fluoro-5-methyl-1H-benzimidazole

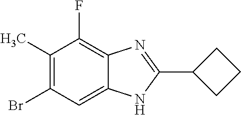 [SMILES: Cc1c(Br)cc2[nH]c(C3CCC3)nc2c1F]

2-Cyclobutyl-4-fluoro-5-methyl-1H-benzimidazole (2.54 g, 12.47 mmol) was dissolved in dichloromethane (40 ml) and admixed with N-bromosuccinimide (2.22 g, 12.47 mmol). The reaction mixture was stirred at RT for 12 h. Subsequently, saturated aqueous ammonium chloride solution was added, the phases were separated and the organic phase was concentrated to dryness. The residue was converted further as the crude product.

### Example 50A

### 2-Cyclobutyl-4-fluoro-5-methyl-6-(4,4,5,5-tetramethyl-1,3,2-dioxaborolan-2-yl)-1H-benzimidazole

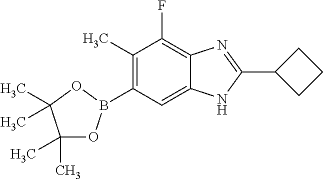 [SMILES: Cc1c(B2OC(C)(C)C(C)(C)O2)cc2[nH]c(C3CCC3)nc2c1F]

A solution of 6-bromo-2-cyclobutyl-4-fluoro-5-methyl-1H-benzimidazole (1.6 g, 5.65 mmol), 4,4,4′,4′,5,5,5′,5′-octamethyl-2,2′-bi-1,3,2-dioxaborolane (2.0 g, 7.88 mmol) and potassium acetate (1.67 g, 17.02 mmol) in 1,4-dioxane (16 ml) was admixed with 1,1′-bis(diphenylphosphino)ferrocene-dichloropalladium(II) (200 mg, 0.27 mmol) and stirred under reflux for 12 h. The mixture was then concentrated under reduced pressure and the residue was purified by chromatography on silica gel (eluent: pentane/ethyl acetate 2/1). This gave 0.5 g (27% of theory) of the title compound.

¹H NMR (400 MHz, CDCl₃): δ ppm=1.37 (s, 12H), 2.07-2.14 (m, 2H), 2.44-2.56 (m, 7H), 3.76-3.80 (m, 1H), 7.63 (s, 1H), 9.38 (s, 1H).

LC-MS (Method 17): Rₜ=3.12 min; MS (ESIpos): m/z=331.1 [M+H]⁺.

### Example 51A

### N-alpha-[(trans-4-{[(tert-Butoxycarbonyl)amino]methyl}cyclohexyl)carbonyl]-4-(2-cyclobutyl-7-fluoro-6-methyl-1H-benzimidazol-5-yl)-N-(2-oxo-2,3-dihydro-1H-benzimidazol-5-yl)-L-phenylalaninamide

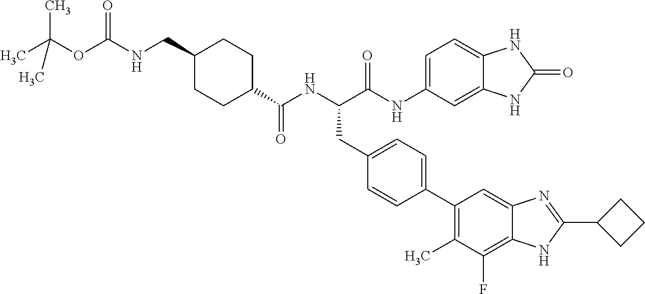

4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (150 mg, 0.24 mmol) and 2-cyclobutyl-4-fluoro-5-methyl-6-(4,4,5,5-tetramethyl-1,3,2-dioxaborolan-2-yl)-1H-benzimidazole (89 mg, 0.27 mmol) were dissolved in dimethyl sulphoxide (3.2 ml) and admixed with 1,1′-bis(diphenylphosphino)ferrocene-dichloropalladium(II) (20 mg, 24 μmol), sodium carbonate (78 mg, 0.73 mmol) and water (0.37 ml, 20.3 mmol). The reaction mixture was stirred at 110° C. for 2 h. The reaction mixture was filtered through deactivated alumina and purified by chromatography via HPLC (Method 8). This gave 65 mg (36% of theory) of the title compound.

LC-MS (Method 5): Rₜ=1.01 min; MS (ESIpos): m/z=738.5 [M+H]⁺.

### Example 52A

### tert-Butyl{[trans-4-({(5S)-3-[4-(6-methyl-1,2,3,4-tetrahydropyrido[2,3-b]pyrazin-7-yl)phenyl]-1-oxo-1-[(2-oxo-2,3-dihydro-1H-benzimidazol-5-yl)amino]propan-2-yl}carbamoyl)cyclohexyl]-methyl}carbamate

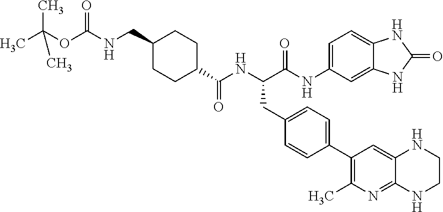

N-alpha-[(trans-4-{[(tert-Butoxycarbonyl)amino]methyl}cyclohexyl)carbonyl]-N-(2-oxo-2,3-dihydro -1H-benzimidazol-5-yl)-4-(4,4,5,5-tetramethyl-1,3,2-dioxaborolan-2-yl)-L-phenyl-alaninamide (170 mg, 0.26 mmol) and 7-bromo-6-methyl-1,2,3,4-tetrahydropyrido[2,3-b]pyrazine (64 mg, 0.28 mmol) were dissolved in dimethyl sulphoxide (2 ml) and admixed with 1,1′-bis(diphenylphosphino)ferrocene-dichloropalladium(II) (21 mg, 26 μmol), sodium carbonate (82 mg, 0.8 mmol) and water (0.4 ml, 21 mmol). The reaction mixture was stirred at 120° C. for 2 h, then filtered through deactivated alumina and purified by chromatography via HPLC (Method 7). This gave 13 mg (8% of theory) of the title compound.

LC-MS (Method 4): 0.92 min; MS (ESIpos): m/z=683.4 [M+H]⁺.

### Example 53A

### Methyl 3-{5-[4-({4-bromo-N-[(trans-4-{[(tert-butoxycarbonyl)amino]methyl}cyclohexyl)carbonyl]-L-phenylalanyl}amino)phenyl]-1H-1,2,4-triazol-3-yl -2,2,3,3-tetrafluoropropanoate

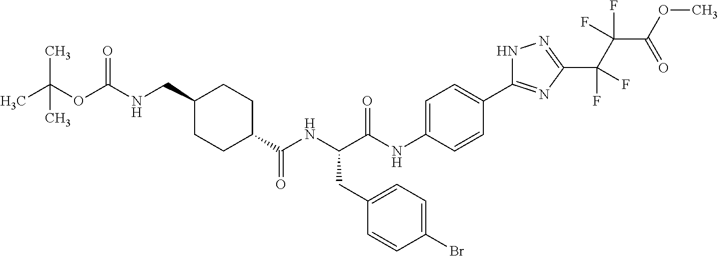 [SMILES: COC(=O)C(F)(F)C(F)(F)c1n[nH]c(-c2ccc(NC(=O)[C@H](Cc3ccc(Br)cc3)NC(=O)[C@H]3CC[C@H](CNC(=O)OC(C)(C)C)CC3)cc2)n1]

4-Bromo-N-[(trans-4-{[(tert-butoxycarbonyl)amino]methyl}cyclohexyl)carbonyl]-L-phenylalanine (1500 mg, 3.1 mmol) and methyl 3-[5-(4-aminophenyl)-1H-1,2,4-triazol-3-yl]-2,2,3,3-tetrafluoropropanoate (1185 mg, 3 7 mmol) were dissolved in 10 ml of pyridine and admixed with 2,4,6-tripropyl-1,3,5,2,4,6-trioxatriphosphinane 2,4,6-trioxide (50% in DMF, 7.2 ml, 12.4 mmol) and stirred at 85° C. for 5 h. Water was added, and the pyridine was removed under reduced pressure. The residue was admixed with dilute aqueous ammonium chloride solution and extracted three times with ethyl acetate. The combined organic phases were washed with water and saturated aqueous sodium chloride solution, and dried over sodium sulphate and under reduced pressure. The residue was purified by chromatography (silica gel, eluent: dichloromethane/methanol=10/1). This gave 1675 mg (63% of theory) of the title compound.

¹H NMR (400 MHz, DMSO-d₆): δ=ppm 0.82 (m, 2H), 1.05-1.30 (m, 3H), 1.36 (s, 9H), 1.53-1.60 (m, 1H), 1.68 (m, 3H), 2.03-2.14 (m, 1H), 2.70-2.78 (m, 2H), 2.80-2.91 (m, 1H), 2.97-3.09 (m, 1H), 3.95 (s, 2H), 4.57-4.72 (m, 1H), 6.72-6.82 (m, 1H), 7.26 (d, 2H), 7.48 (d, 2H), 7.73-7.81 (m, 2H), 7.96 (d, 2H), 8.15 (d, 1H), 10.44 (s, 1H), 15.19 (br. s, 1H).

LC-MS (Method 1): Rₜ=1.23 min; MS (ESIpos): m/z=785.4 [M+H]⁺.

### Example 54A

### Methyl 2,2,3,3-tetrafluoro-3-[5-(4-nitrophenyl)-1H-1,2,4-triazol-3-yl]propanoate

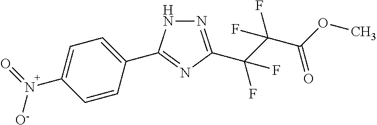

2,2,3,3-Tetrafluoro-3-[5-(4-nitrophenyl)-1H-1,2,4-triazol-3-yl]propanoic acid (30.3 g, 90.8 mmol) was dissolved in methanol (500 ml) and admixed with concentrated sulphuric acid (3 ml). The mixture was stirred at 65° C. for 22 h. Then concentrated sulphuric acid (5 ml) was added and the mixture was stirred once again at 65° C. for 22 h. Sodium hydrogencarbonate was added at RT to pH=7, the mixture was filtered and the solvent was removed under reduced pressure. The residue was stirred in petroleum ether and diethyl ether and then filtered. This gave 31.6 g (77% of theory) of the title compound.

LC-MS (Method 1): Rₜ=0.96 min; MS (ESIpos): m/z=349.1 [M+H]⁺.

### Example 55A

### Methyl 3-[5-(4-aminophenyl)-1H-1,2,4-triazol-3-yl]-2,2,3,3-tetrafluoropropanoate

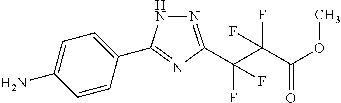 [SMILES: COC(=O)C(F)(F)C(F)(F)c1n[nH]c(-c2ccc(N)cc2)n1]

Methyl 2,2,3,3-tetrafluoro-3-[5-(4-nitrophenyl)-1H-1,2,4-triazol-3-yl]propanoate (24.0 g, 68.9 mmol) was initially charged in THF (370 ml) and admixed with palladium/charcoal (10%, 50% water-moist) under an argon atmosphere. Hydrogenation was effected with hydrogen (1 bar) at RT for 18 h. The mixture was filtered through kieselguhr and washed with dichloromethane/methanol 9:1. The filtrate was concentrated and the residue was dried under reduced pressure. This gave 21.7 g (99% of theory) of the title compound.

LC-MS (Method 1): Rₜ=0.78 min; MS (ESIpos): m/z=319.1 [M+H]⁺.

### Example 56A

### 3-{5-[4-({N-[(trans-4-{[(tert-Butoxycarbonyl)amino]methyl}cyclohexyl)carbonyl]-4-(1-oxo-2,3-dihydro-1H-isoindol-5-yl)-L-phenylalany}amino)phenyl]-1H-1,2,4-triazol-3-yl -2,2,3,3-tetrafluoropropanoic acid

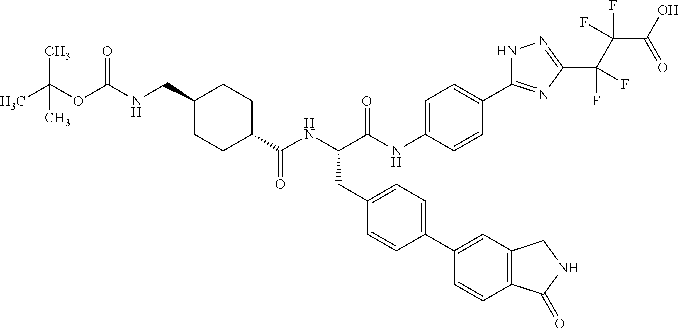 [SMILES: CC(C)(C)OC(=O)NC[C@H]1CC[C@H](C(=O)N[C@@H](Cc2ccc(-c3ccc4c(c3)CNC4=O)cc2)C(=O)Nc2ccc(-c3nc(C(F)(F)C(F)(F)C(=O)O)n[nH]3)cc2)CC1]

Methyl 3-{5-[4-({4-bromo-N-[(trans-4-{[(tert-butoxycarbonyl)amino]methyl}cyclohexyl)-carbonyl]-L-phenylalanyl}amino)phenyl]-1H-1,2,4-triazol-3-yl]-2,2,3,3-tetrafluoropropanoate (125 mg, 0.16 mmol) and 5-(4,4,5,5-tetramethyl-1,3,2-dioxaborolan-2-yl)isoindolin-1-one (50 mg, 0.19 mmol) were dissolved in 1.3 ml dimethylformamide and admixed with saturated aqueous sodium carbonate solution (2M, 0.16 ml, 0.32 mmol) and degassed. After adding 12 mg (0.02 mmol) of 1,1′-bis(diphenylphosphino)ferrocenepalladium(II) chloride, the reaction mixture was stirred at 120° C. for 30 min. The reaction solution was filtered through a Millipore syringe filter and purified via preparative HPLC (eluent: acetonitrile/water with 0.1% trifluoroacetic acid (gradient)). This gave 77 mg (42% of theory) of the title compound.

LC-MS (Method 1): Rₜ=0.94 min; MS (ESIpos): m/z=822.5 [M+H]⁺.

### Example 57A

### N-alpha-[(trans-4-{[(tert-Butoxycarbonyl)amino]methyl}cyclohexyl)carbonyl]-4-(5-methyl-1H-benzimidazol-6-yl)-N-(3-oxo-2,3-dihydro-1H-indazol-6-yl)-L-phenylalaninamide

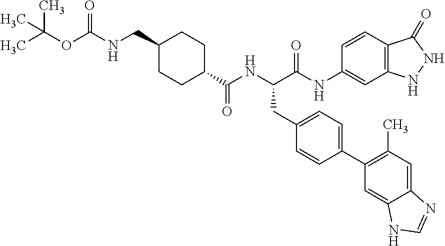 [SMILES: Cc1cc2nc[nH]c2cc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

To a solution of 4-bromo-N-alpha-[(trans-4-{[(tert-butoxycarbonyl)amino]methyl}-cyclohexyl)carbonyl]-N-(3-oxo-2,3-dihydro-1H-indazol-6-yl)-L-phenylalaninamide (150 mg, 0.244 mmol) and tert-butyl 5-methyl-6-(4,4,5,5-tetramethyl-1,3,2-dioxaborolan-2-yl)-1H-benzimidazole-1-carboxylate (122.4 mg, 0.342 mmol) in 1,2-dimethoxyethane (2 ml) were added ethanol (0.8 ml), 2N aqueous sodium carbonate solution (0.24 ml, 0.49 mmol) and [1,1-bis(diphenylphosphino)-ferrocene]dichloropalladium-dichloromethane complex (10 mg, 0.012 mmol). The mixture was then stirred at reflux (oil bath temperature 100° C.) for 8 h. The reaction mixture was concentrated on a rotary evaporator and the residue was dissolved in DMSO/water/acetonitrile (about 5 ml). The solution was filtered through a Millipore filter and purified by preparative HPLC (eluent: gradient of acetonitrile/water with 0.1% trifluoroacetic acid). This gave 48.3 mg (19% of theory) of the title compound (about 25% without Boc protecting group).

¹H NMR (400 MHz, DMSO-d₆): δ ppm 0.74-0.93 (m, 2H), 1.09-1.30 (m, 4H), 1.32-1.42 (m, 9H), 1.61 (s, 11 H), 2.09-2.19 (m, 1H), 2.09-2.19 (m, 1H), 2.24-2.31 (m, 3H), 2.60-2.69 (m, 1H), 2.75 (s, 2H), 2.90-3.01 (m, 1H), 3.09-3.16 (m, 1H), 4.72-4.82 (m, 1H), 6.71-6.82 (m, 1H), 7.29 (d, 2H), 7.35-7.45 (m, 3H), 7.53 (s, 1H), 7.62 (s, 1H), 7.71 (s, 1H), 8.11-8.26 (m, 1H), 8.52-8.63 (m, 1H), 9.29-9.38 (m, 1H), 10.37-10.48 (m, 1H).

LC-MS (Method 1): Rₜ=0.87 min; MS (ESIneg): m/z=764 [M−H]⁻.

### Example 58A

### N-alpha-[(trans-4-{[(tert-Butoxycarbonyl)amino]methyl}cyclohexyl)carbonyl]-4-[2-isopropyl-5-(trifluoromethyl)-1H-benzimidazol-6-yl]-N-(2-oxo-2,3-dihydro-1H-benzimidazol-5-yl)-L-phenylalaninamide

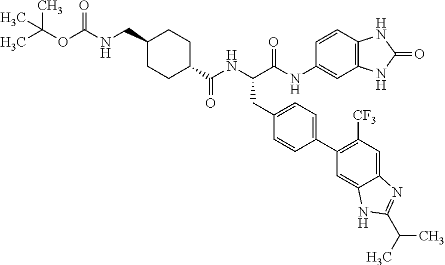 [SMILES: CC(C)c1nc2cc(C(F)(F)F)c(-c3ccc(C[C@H](NC(=O)[C@H]4CC[C@H](CNC(=O)OC(C)(C)C)CC4)C(=O)Nc4ccc5[nH]c(=O)[nH]c5c4)cc3)cc2[nH]1]

To a solution of 4-bromo-N-alpha-[(trans-4-{[(tert-butoxycarbonyl)amino]methyl}-cyclohexyl)carbonyl]-N-(2-oxo-2,3-dihydro-1H-benzimidazol-5-yl)-L-phenylalaninamide (80 mg, 0.13 mmol) and 2-isopropyl-6-(4,4,5,5-tetramethyl-1,3,2-dioxaborolan-2-yl)-5-(trifluoromethyl)-1H-benzimidazole (51 mg, 0.143 mmol) in 1,2-dimethoxyethane (2 ml) were added ethanol (0.66 ml), 2N aqueous sodium carbonate solution (0.13 ml, 0.26 mmol) and [1,1-bis(diphenylphosphino)-ferrocene]dichloropalladium-dichloromethane complex (10.6 mg, 0.013 mmol). The mixture was then stirred at reflux (oil bath temperature 100° C.) for 3 h. The reaction mixture was concentrated on a rotary evaporator and the residue was dissolved in DMSO/water/acetonitrile (about 5 ml). The solution was filtered through a Millipore filter and purified by preparative HPLC (eluent: gradient of acetonitrile/water with 0.1% trifluoroacetic acid). This gave 18.6 mg (19% of theory) of the title compound.

LC-MS (Method 1): Rₜ=0.88 min; MS (ESIneg): m/z=760 [M−H]⁻.

### Example 59A

### tert-Butyl 2-(4-chloro-2-iodobenzoyl)-2-isopropylhydrazinecarboxylate

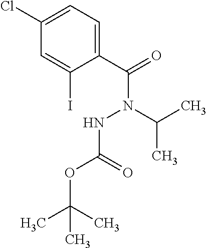 [SMILES: CC(C)N(NC(=O)OC(C)(C)C)C(=O)c1ccc(Cl)cc1I]

A solution of 4-chloro-2-iodobenzoic acid (500 mg, 1.77 mmol) in ethyl acetate (3.5 ml) was admixed with tert-butyl 2-isopropylhydrazinecarboxylate (339 mg, 1.95 mmol) and N,N-diisopropylethylamine (0.93 ml, 5.31 mmol). The solution was admixed with a 2,4,6-tripropyl-1,3,5,2,4,6-trioxatriphosphinane 2,4,6-trioxide solution (50% in DMF, 3.1 ml, 5.31 mmol) and then stirred at RT overnight. The reaction mixture was admixed with DMSO (about 3 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 411.7 mg (53% of theory) of the title compound.

LC-MS (Method 1): Rₜ=1.11 min; MS (ESIneg): m/z=437 [M−H]⁻.

### Example 60A

### tert-Butyl 6-chloro-2-isopropyl-3-oxo-2,3-dihydro-1H-indazole-1-carboxylate

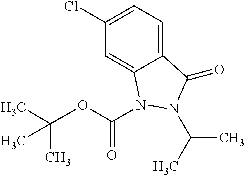

Under argon, caesium carbonate (424 mg, 1.30 mmol), 1,10-phenanthroline (16.8 mg, 0.093 mmol) and copper(I) iodide (1.8 mg, 0.09 mmol) were initially charged in a flask, and a solution of tert-butyl 2-(4-chloro-2-iodobenzoyl)-2-isopropylhydrazinecarboxylate (407.8 mg, 0.93 mmol) in DMF (1.2 ml) was added thereto. The reaction mixture was stirred at RT for 4 h, then added to water and extracted with ethyl acetate. The combined organic phases were washed with saturated aqueous sodium chloride solution and dried over sodium sulphate, and the solvent was removed on a rotary evaporator. The residue was purified by flash chromatography (eluent: ethyl acetate/cyclohexane, 1:5). The product-containing fractions were collected and the solvent was removed on a rotary evaporator. This gave 196.2 mg (68% of theory) of the title compound.

LC-MS (Method 1): Rₜ=1.22 min; MS (ESIpos): m/z=311 [M+H]⁺.

### Example 61A

### tert-Butyl 6-{4-[(2S)-2-[(trans-4-{[(tert-butoxycarbonyl)amino]methyl}cyclohexyl)-carbonyl]amino}-3-(1H-indazol-6-ylamino)-3-oxopropyl]phenyl}-2-isopropyl-3-oxo-2,3-dihydro-1H-indazole-1-carboxylate

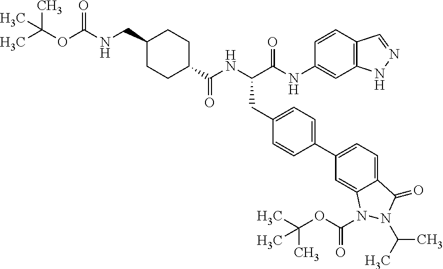 [SMILES: CC(C)n1c(=O)c2ccc(-c3ccc(C[C@H](NC(=O)[C@H]4CC[C@H](CNC(=O)OC(C)(C)C)CC4)C(=O)Nc4ccc5cn[nH]c5c4)cc3)cc2n1C(=O)OC(C)(C)C]

To a solution of N-alpha-[(trans-4-{[(tert-butoxy carbonyl)amino]methyl}cyclohexyl)carbonyl]-N-1H-indazol-6-yl-4-(4,4,5,5-tetramethyl-1,3,2-dioxaborolan-2-yl)-L-phenylalaninamide (50 mg, 0.077 mmol) and tert-butyl 6-chloro-2-isopropyl-3-oxo-2,3-dihydro-1H-indazole-1-carboxylate (26.4 mg, 0.085 mmol) in 1,2-dimethoxyethane (1 ml) were added ethanol (0.33 ml), 2N aqueous sodium carbonate solution (0.077 ml, 0.16 mmol) and [1,1-bis(diphenylphosphino)-ferrocene]dichloropalladium-dichloromethane complex (6.3 mg, 0.008 mmol). The mixture was stirred at reflux (oil bath temperature 100° C.) for 3 h. The reaction mixture was concentrated on a rotary evaporator and the residue was dissolved in DMSO/water/acetonitrile (about 5 ml). The solution was filtered through a Millipore filter and purified by preparative HPLC (eluent: gradient of acetonitrile/water with 0.1% trifluoroacetic acid). This gave 15.8 mg (19% of theory) of the title compound.

LC-MS (Method 1): Rₜ=1.19 min; MS (ESIneg): m/z=792 [M−H]⁻.

### Example 62A

### tert-Butyl 6-[4-{(2S)-2-{[(trans-4-{[(tert-butoxycarbonyl)amino]methyl}cyclohexyl)carbonyl]-amino}-3-oxo-3-[(2-oxo-2,3-dihydro-1H-benzimidazol-5-yl)amino]propyl}phenyl)-2-isopropyl-3-oxo-2,3-dihydro-1H-indazole-1-carboxylate

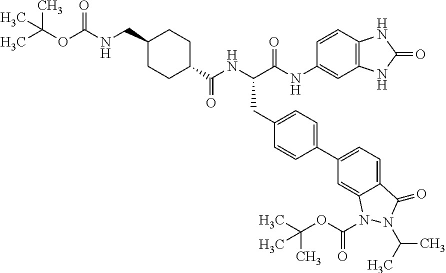 [SMILES: CC(C)n1c(=O)c2ccc(-c3ccc(C[C@H](NC(=O)[C@H]4CC[C@H](CNC(=O)OC(C)(C)C)CC4)C(=O)Nc4ccc5[nH]c(=O)[nH]c5c4)cc3)cc2n1C(=O)OC(C)(C)C]

To a solution of N-alpha-[(trans-4-{[(tert-butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenylalaninamide (150 mg, 0.227 mmol) and tert-butyl 6-chloro-2-isopropyl-3-oxo-2,3-dihydro-1H-indazole-1-carboxylate (77.5 mg, 0.249 mmol) in 1,2-dimethoxyethane (3 ml) were added ethanol (1 ml), 2N aqueous sodium carbonate solution (0.23 ml, 0.453 mmol) and [1,1-bis(diphenylphosphino)ferrocene]dichloropalladium-dichloromethane complex (18.5 mg, 0.023 mmol). The mixture was stirred at reflux (oil bath temperature 100° C.) for 3 h. The reaction mixture was concentrated on a rotary evaporator and the residue was dissolved in DMSO/water/acetonitrile (about 5 ml). The solution was filtered through a Millipore filter and purified by preparative HPLC (eluent: gradient of acetonitrile/water with 0.1% trifluoroacetic acid). This gave 52.3 mg (28% of theory) of the title compound.

LC-MS (Method 1): Rₜ=1.14 min; MS (ESIneg): m/z=808 [M−H]⁻.

### Example 63A

### tert-Butyl 2-(4-bromo-2-fluoro-5-methylbenzoyl)-2-isopropylhydrazinecarboxylate

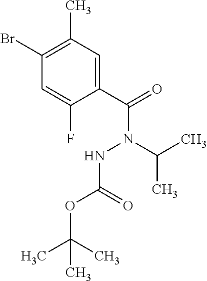 [SMILES: Cc1cc(C(=O)N(NC(=O)OC(C)(C)C)C(C)C)c(F)cc1Br]

A solution of 4-bromo-2-fluoro-5-methylbenzoic acid (500 mg, 2.15 mmol) and DMF (0.05 ml) in dichloromethane (5 ml) was admixed with oxalyl chloride and stirred at RT for 1 h. The reaction mixture was concentrated on a rotary evaporator and coevacuated once again with dichloromethane. The residue was dissolved in THF (7 ml) and admixed with N,N-diisopropylethylamine (0.45 ml, 2.57 mmol) and tert-butyl 2-isopropylhydrazinecarboxylate (411 mg, 2.36 mmol). The solution was stirred at RT for 1 h. The reaction mixture was diluted with water and acidified slightly with 1N hydrochloric acid. The mixture was extracted with ethyl acetate and the combined organic phases were washed with saturated aqueous sodium chloride solution, dried over sodium sulphate, filtered and concentrated on a rotary evaporator. This gave 825 mg (96% of theory) of the title compound, which was used further without purification.

LC-MS (Method 1): Rₜ=1.11 min; MS (ESIneg): m/z=387 [M−H]⁻.

### Example 64A

### 6-Bromo-2-isopropyl-5-methyl-1,2-dihydro-3H-indazol-3-one

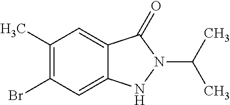

A solution of tert-butyl 2-(4-bromo-2-fluoro-5-methylbenzoyl)-2-isopropylhydrazinecarboxylate (785 mg, 2.02 mmol) in DMF (8 ml) was admixed with sodium hydride in oil (60%, 97 mg, 2.42 mmol) and stirred at RT overnight. The reaction mixture was admixed once again with sodium hydride in oil (60%, 81 mg, 2.02 mmol) and stirred at RT for a further 6 days. Additional sodium hydride in oil (60%, 81 mg, 2.02 mmol) was added thereto and the mixture was stirred for a further 4 days. The reaction was then admixed with water (150 ml) and extracted twice with ethyl acetate. The combined organic phases were washed once with water and concentrated on a rotary evaporator. The residue was dissolved in DMSO/water/acetonitrile (about 5 ml). The solution was filtered through a Millipore filter and purified by preparative HPLC (eluent: gradient of acetonitrile/water with 0.1% trifluoroacetic acid). This gave 289 mg (53% of theory) of the title compound.

LC-MS (Method 1): Rₜ=0.79 min; MS (ESIpos): m/z=269 [M+H]⁺.

### Example 65A

### N-alpha-[(trans-4-{[(tert-Butoxycarbonyl)amino]methyl}cyclohexyl)carbonyl]-4-(2-isopropyl-5-methyl-3-oxo-2,3-dihydro-1H-indazol-6-yl)-N-(2-oxo-2,3-dihydro-1H-benzimidazol-5-yl)-L-phenylalaninamide

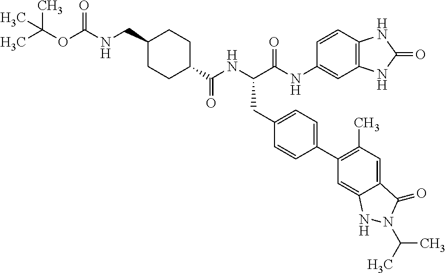 [SMILES: Cc1cc2c(=O)n(C(C)C)[nH]c2cc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

To a solution of N-alpha-[(trans-4-{[(tert-butoxy carbonyl)amino]methyl}cyclohexyl)-carbonyl]-N-(2-oxo-2,3-dihydro -1H-benzimidazol-5-yl)-4-(4,4,5,5-tetramethyl-1,3,2-dioxaborolan-2-yl)-L-phenylalaninamide (100 mg, 0.151 mmol) and 6-bromo-2-isopropyl-5-methyl-1,2-dihydro-3H-indazol-3-one (44.7 mg, 0.166 mmol) in 1,2-dimethoxyethane (2 ml) were added ethanol (0.7 ml), 2N aqueous sodium carbonate solution (0.15 ml, 0.30 mmol) and [1,1-bis(diphenylphosphino)-ferrocene]dichloropalladium-dichloromethane complex (12.3 mg, 0.015 mmol). The mixture was stirred at reflux (oil bath temperature 110° C.) for 4 h. The reaction mixture was concentrated on a rotary evaporator and the residue was dissolved in DMSO/water/acetonitrile (about 5 ml). The solution was filtered through a Millipore filter and purified by preparative HPLC (eluent: gradient of acetonitrile/water with 0.1% trifluoroacetic acid). This gave 70 mg (62% of theory) of the title compound.

LC-MS (Method 1): Rₜ=0.86 min; MS (ESIneg): m/z=722 [M−H]⁻.

### Example 66A

### 6-Bromo-4,5-difluoro-2-isopropyl-1H-benzimidazole

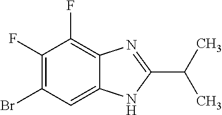

A solution of 5-bromo-3,4-difluorobenzene-1,2-diamine (400 mg, 1.79 mmol) in ethanol (8 ml) was admixed with 2-methylpropionaldehyde (0.20 ml, 2.15 mmol) and cobalt dihydroxide (16.7 mg, 0.18 mmol) and stirred at RT overnight. The reaction mixture was concentrated on a rotary evaporator and the residue was dissolved in DMSO/water/acetonitrile. The solution was filtered through a Millipore filter and purified by preparative HPLC (eluent: gradient of acetonitrile/water with 0.1% trifluoroacetic acid). This gave 270.1 mg (55% of theory) of the title compound.

LC-MS (Method 1): Rₜ=0.87 min; MS (ESIpos): m/z=275 [M+H]⁺.

### Example 67A

### N-alpha-[(trans-4-{[(tert-Butoxycarbonyl)amino]methyl}cyclohexyl)carbonyl]-4-(4,5-difluoro-2-isopropyl-1H-benzimidazol-6-yl)-N-(2-oxo-2,3-dihydro-1H-benzimidazol-5-yl)-L-phenylalaninamide

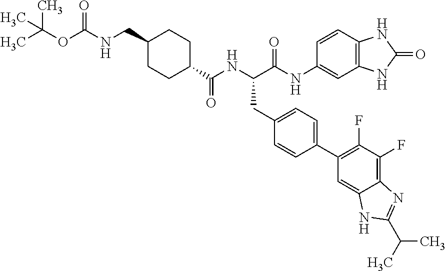 [SMILES: CC(C)c1nc2c(F)c(F)c(-c3ccc(C[C@H](NC(=O)[C@H]4CC[C@H](CNC(=O)OC(C)(C)C)CC4)C(=O)Nc4ccc5[nH]c(=O)[nH]c5c4)cc3)cc2[nH]1]

To a solution of N-alpha-[(trans-4-{[(tert-butoxycarbonyl)amino]methyl}cyclohexyl)carbonyl]-N-(2-oxo-2,3-dihydro -1H-benzimidazol-5-yl)-4-(4,4,5,5-tetramethyl-1,3,2-dioxaborolan-2-yl)-L-phenylalaninamide (100 mg, 0.151 mmol) and 6-bromo-4,5-difluoro-2-isopropyl-1H-benzimidazole (45.7 mg, 0.166 mmol) in 1,2-dimethoxyethane (2 ml) were added ethanol (0.7 ml), 2N aqueous sodium carbonate solution (0.15 ml, 0.30 mmol) and [1,1-bis(diphenylphosphino)-ferrocene]dichloropalladium-dichloromethane complex (12.3 mg, 0.015 mmol). The mixture was stirred at reflux (oil bath temperature 110° C.) for 4 h. The reaction mixture was concentrated on a rotary evaporator and the residue was dissolved in DMSO/water/acetonitrile (about 5 ml). The solution was filtered through a Millipore filter and purified by preparative HPLC (eluent: gradient of acetonitrile/water with 0.1% trifluoroacetic acid). This gave 47 mg (36% of theory) of the title compound.

LC-MS (Method 1): Rₜ=0.91 min; MS (ESIneg): m/z=728 [M−H]⁻.

### Example 68A

### 6-Bromo-2-cyclopropyl-7-methyl[1,2,4]triazolo[1,5-a]pyridine

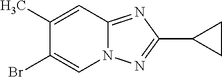

A solution of water (0.32 ml) and trifluoroacetic acid (4.2 ml) was cooled to −5° C., admixed with ethyl N-[(mesitylsulphonyl)oxy]ethanimidate (2.29 g, 8.02 mmol) and stirred at RT for 1 h. The reaction solution was admixed with ice-water (20 ml) and then extracted with dichloromethane (20 ml). The organic phase was dried over sodium sulphate and filtered, and the solution obtained was added dropwise at 0° C. to a solution of 5-bromo-4-methylpyridin-2-amine (1 g, 1.79 mmol) in dichloromethane (15 ml). The mixture was stirred at RT for 1 h and then admixed with diethyl ether (20 ml). The precipitated solid was filtered off and washed with diethyl ether. The solid was dried under high vacuum. This gave 1.22 g (57% of theory) of 1,2-diamino-5-bromo-4-methylpyridinium 2,4,6-trimethylbenzenesulphonate. A solution of 1,2-diamino-5-bromo-4-methylpyridinium 2,4,6-trimethylbenzenesulphonate (580 mg, 1.44 mmol) and cyclopropylcarboxylic anhydride (1.33 g, 8.65 mmol) in pyridine (2.5 ml) was stirred at 100° C. for 8 h. The reaction mixture was concentrated on a rotary evaporator and the residue was dissolved in DMSO/water/acetonitrile. The solution was filtered through a Millipore filter and purified by preparative HPLC (eluent: gradient of acetonitrile/water with 0.1% trifluoroacetic acid). This gave 279 mg (69% of theory) of the title compound.

LC-MS (Method 1): Rₜ=0.84 min; MS (ESIpos): m/z=252 [M+H]⁺.

### Example 69A

### N-alpha-[(trans-4-{[(tert-Butoxycarbonyl)amino]methyl}cyclohexyl)carbonyl]4-(2-cyclopropyl-7-methyl[1,2,4]triazolo[1,5-a]pyridin-6-yl)-N-(2-oxo-2,3-dihydro-1H-benzimidazol-5-yl)-L-phenylalaninamide

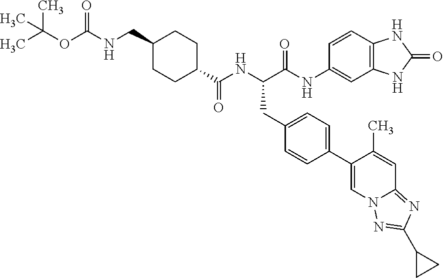 [SMILES: Cc1cc2nc(C3CC3)nn2cc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

To a solution of N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenylalaninamide (100 mg, 0.15 mmol) and 6-bromo-2-cyclopropyl-7-methyl[1,2,4]triazolo[1,5-a]pyridine (42 mg, 0.17 mmol) in 1,2-dimethoxyethane (2 ml) were added ethanol (0.7 ml), 2N aqueous sodium carbonate solution (0.15 ml, 0.30 mmol) and [1,1-bis(diphenylphosphino)-ferrocene]dichloropalladium-dichloromethane complex (12.3 mg, 0.015 mmol). The mixture was stirred at reflux (oil bath temperature 110° C.) for 4 h. The reaction mixture was diluted with DMSO and acetonitrile, filtered through a Millipore filter and purified by preparative HPLC (eluent: gradient of acetonitrile/water with 0.1% trifluoroacetic acid). This gave 68 mg (62% of theory) of the title compound.

LC-MS (Method 1): Rₜ=0.92 min; MS (ESIneg): m/z=705 [M−H]⁻.

### Example 70A

### 5-Chloro-2-isopropyl-6-methyl-3H-imidazo[4,5-b]pyridine

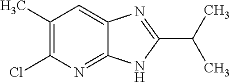

A solution of 6-chloro-5-methylpyridine-2,3-diamine (400 mg, 2.28 mmol) in ethanol (8 ml) was admixed with 2-methylpropionaldehyde (0.25 ml, 2.74 mmol) and cobalt dihydroxide (21.2 mg, 0.23 mmol) and stirred at RT overnight. The reaction mixture was concentrated on a rotary evaporator and the residue was dissolved in DMSO/water/acetonitrile. The solution was filtered through a Millipore filter and purified by preparative HPLC (eluent: gradient of acetonitrile/water with 0.1% trifluoroacetic acid). This gave 323.4 mg (68% of theory) of the title compound.

LC-MS (Method 1): Rₜ=0.63 min; MS (ESIpos): m/z=210 [M+H]⁺.

### Example 71A

### N-alpha-[(trans-4-{[(tert-Butoxycarbonyl)amino]methyl}cyclohexyl)carbonyl]-4-(2-isopropyl-6-methyl-3H-imidazo[4,5-b]pyridin-5-yl)-N-(2-oxo-2,3-dihydro-1H-benzimidazol-5-yl)-L-phenylalaninamide

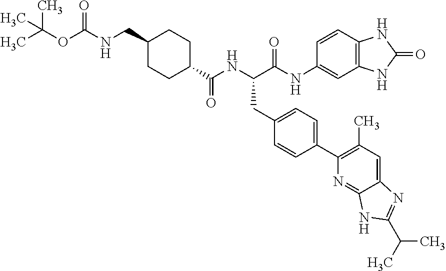 [SMILES: Cc1cc2nc(C(C)C)[nH]c2nc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

To a solution of N-alpha-[(trans-4-{[(tert-Butoxycarbonyl)amino]methyl}cyclohexyl)-carbonyl]-N-(2-oxo-2,3-dihydro-1H-benzimidazol-5-yl)-4-(4,4,5,5-tetramethyl-1,3,2-dioxaborolan-2-yl)-L-phenylalaninamide (150 mg, 0.23 mmol) and 5-chloro-2-isopropyl-6-methyl-3H-imidazo[4,5-b]pyridine (52 mg, 0.25 mmol) in 1,2-dimethoxyethane (3 ml) were added ethanol (1 ml), 2N aqueous sodium carbonate solution (0.23 ml, 0.45 mmol) and [1,1-bis(diphenylphosphino)-ferrocene]dichloropalladium-dichloromethane complex (18.5 mg, 0.023 mmol). The mixture was stirred at 120° C. in a microwave for 3 h. The reaction mixture was diluted with DMSO/water/acetonitrile, filtered through a Millipore filter and purified by preparative HPLC (eluent: gradient of acetonitrile/water with 0.1% trifluoroacetic acid). This gave 89 mg (37% of theory) of the title compound.

LC-MS (Method 1): Rₜ=0.76 min; MS (ESIneg): m/z=707 [M−H]⁻.

### Example 72A

### N-alpha-[(trans-4-{[(tert-Butoxycarbonyl)amino]methyl}cyclohexyl)carbonyl]-4-(2-isopropyl-6-methyl-1H-benzimidazol-5-yl)-N-(2-oxo-2,3-dihydro-1H-benzimidazol-5-yl)phenylalaninamide (Enantiomer Mixture)

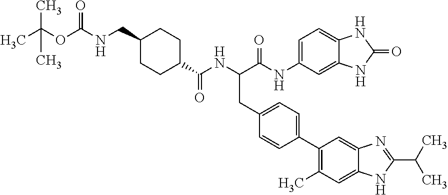

N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenyl-alaninamide (2.0 g, 3.0 mmol) and 5-bromo-2-isopropyl-6-methyl-1H-benzimidazole (842 mg, 3.33 mmol) were dissolved in dimethyl sulphoxide (18 ml) and admixed with 1,1′-bis(diphenylphosphino)ferrocene-dichloropalladium(II) (123 mg, 150 μmol), sodium carbonate (961 mg, 9.1 mmol) and water (4.6 ml, 252 mmol). The reaction mixture was stirred at 120° C. for 2 h. The reaction mixture was admixed with water, and the residue was filtered off, dried and purified by chromatography via silica gel (Biotage Isolera, SNAP NH 375 g, eluent: hexane/ethyl acetate/methanol). This gave 391 mg (18% of theory) of the title compound.

¹H NMR (300 MHz, DMSO-d₆): δ=ppm 0.72-0.89 (m, 2H), 1.08-1.28 (m, 3H), 1.34 (s, 9H), 1.48-1.71 (m, 4H), 2.04-2.15 (m, 1H), 2.20 (s, 3H), 2.70-2.77 (m, 2H), 2.84-2.94 (m, 1H), 3.02-3.16 (m, 2H), 4.63-4.74 (m, 1H), 6.74-6.79 (m, 1H), 6.80-6.85 (m, 1H), 6.98-7.03 (m, 1H), 7.21 (d, 2H), 7.28-7.36 (m, 2H), 7.43 (s, 1H), 8.04-8.10 (m, 1H), 8.18 (s, 1H), 9.91-9.99 (m, 1H), 10.45-10.50 (m, 1H), 10.52-10.58 (m, 1H).

LC-MS (Method 4): Rₜ=0.92 min; MS (ESIpos): m/z=708.5 [M+H]⁺.

### Example 73A

### N-alpha-[(trans-4-{[(tert-Butoxycarbonyl)amino]methyl}cyclohexyl)carbonyl]-4-(2-isopropyl-6-methyl-1H-benzimidazol-5-yl)-N-(2-oxo-2,3-dihydro-1H-benzimidazol-5-yl)-D-phenylalaninamide (Enantiomer 1)

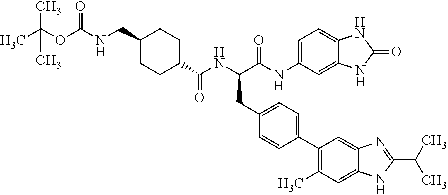

Enantiomer separation of 72 mg of the enantiomer mixture from Example 72A gave 16 mg of the title compound (enantiomer 1).

Chiral analytical HPLC: Rₜ=2.6 min; chiral HPLC: Rₜ=6.8-7.6 min; 100% ee.

Separation method (system: Agilent: Prep 1200, 2× Prep Pump, DLA, MWD, Prep FC): column: Chiralpak IC 5 μm 250 mm×20 mm; eluent: hexane/ethanol 70:30+0.1% diethylamine; temperature: RT; flow rate: 15 ml/min; UV detection: 254 nm.

Analysis (Agilent 1260/Agilent 1290): column: Chiralpak IC 3 μm 100 mm×4.6 mm; eluent: hexane/ethanol 70:30+0.1% diethylamine; temperature: 25° C.; flow rate: 1 ml/min; UV detection: 254 nm.

### Example 73A

### N-alpha-[(trans-4-{[(tert-Butoxycarbonyl)amino]methyl}cyclohexyl)carbonyl]-4-(2-isopropyl-6-methyl-1H-benzimidazol-5-yl)-N-(2-oxo-2,3-dihydro-1H-benzimidazol-5-yl)-L-phenylalaninamide (Enantiomer 2)

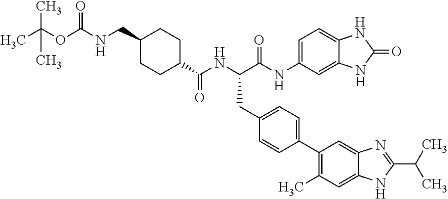

Enantiomer separation of 72 mg of the enantiomer mixture from Example 72A gave 18 mg of the title compound (enantiomer 2).

Chiral analytical HPLC: Rₜ=3.8 min; chiral HPLC: Rₜ=8.3-9.2 min; 94.7% ee.

Separation method (system: Agilent: Prep 1200, 2× Prep Pump, DLA, MWD, Prep FC): column: Chiralpak IC 5 μm 250 mm×20 mm; eluent: hexane/ethanol 70:30+0.1% diethylamine; temperature: RT; flow rate: 15 ml/min; UV detection: 254 nm.

Analysis (Agilent 1260/Agilent 1290): column: Chiralpak IC 3 μm 100 mm×4.6 mm; eluent: hexane/ethanol 70:30+0.1% diethylamine; temperature: 25° C.; flow rate: 1 ml/min; UV detection: 254 nm.

### Working Examples

### Example 1

### N-alpha-{[trans-4-(Aminomethyl)cyclohexyl]carbonyl}-4-(5-methyl-1H-benzimidazol-6-yl)-N-[4-(2H-tetrazol-5-yl)phenyl]-L-phenylalaninamide hydrochloride

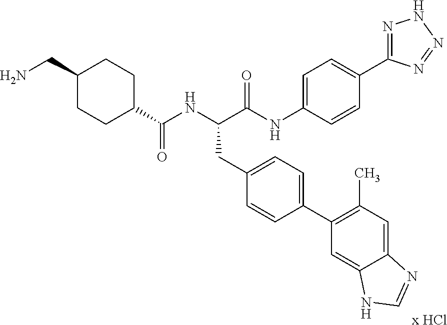

A suspension of 34 mg (0.05 mmol) of N-alpha-[(trans-4-{[(tert-butoxycarbonyl)amino]-methyl}cyclohexyl)carbonyl]-4-(5-methyl-1H-benzimidazol-6-yl)-N-[4-(2H-tetrazol-5-yl)phenyl]-L-phenylalaninamide in 1.5 ml of 1,4-dioxane was admixed with 0.13 ml (0 5 mmol) of 4M hydrogen chloride in 1,4-dioxane and stirred at RT for 16 h. After further addition of 0.04 ml (0.15 mmol) of 4M hydrogen chloride in 1,4-dioxane and stirring at RT for 48 h, the reaction mixture was admixed with 5 ml of acetonitrile, and the precipitate formed was filtered off with suction and dried under high vacuum. This gave 27 mg (83% of theory) of the title compound.

¹H NMR (300 MHz, DMSO-d₆): δ=ppm 0.81-0.98 (m, 2H), 1.06-1.35 (m, 2H), 1.38-1.61 (m, 2H), 1.67-1.80 (m, 3H), 2.05-2.18 (m, 1H), 2.25 (s, 3H), 2.56-2.66 (m, 2H), 2.94 (dd, 1H), 3.12 (dd, 1H), 4.68-4.80 (m, 1H), 7.26 (d, 2H), 7.37 (d, 2H), 7.49 (s, 1H), 7.68 (s, 1H), 7.80 (m, 5H), 7.97 (d, 2H), 8.24 (d, 1H), 9.37 (s, 1H), 10.50 (s, 1H).

LC-MS (Method 4): Rₜ=0.6 min; MS (ESIpos): m/z=578 [M+H−HCl]⁻.

### Example 2

### N-alpha-{[trans-4-(Aminomethyl)cyclohexyl]carbonyl}-4-(1H-pyrrolo[2,3-b]pyridin-5-yl)-N-[4-(2H-tetrazol-5-yl)phenyl]-L-phenylalaninamide hydrochloride

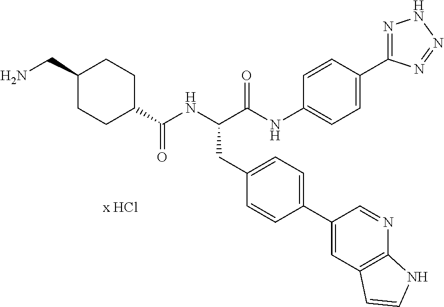

A solution of 88 mg (0.13 mmol) of N-alpha-[(trans-4-{[(tert-butoxycarbonyl)amino]methyl}-cyclohexyl)carbonyl]-4-(1H-pyrrolo[2,3-b]pyridin-5-yl)-N-[4-(2H-tetrazol-5-yl)phenyl]-L-phenylalaninamide trifluoroacetate in 3.5 ml of dioxane was admixed with 0.5 ml (2.00 mmol) of 4M hydrogen chloride in 1,4-dioxane and stirred at RT for 16 h. The solvent was removed on a rotary evaporator. The solid formed was dried under high vacuum. 77 mg (92% of theory) of the title compound were obtained.

¹H NMR (400 MHz, DMSO-d₆): δ=ppm 0.85-1.01 (m, 2H), 1.13-1.37 (m, 2H), 1.40-1.68 (m, 2H), 1.68-1.83 (m, 3H), 2.11-2.21 (m, 1H), 2.58-2.68 (m, 2H), 2.90-3.00 (m, 1H), 3.08-3.17 (m, 1H), 4.67-4.77 (m, 1H), 6.64 (s, 1H), 7.44 (d, 2H), 7.55 (s, 1H), 7.65 (d, 2H), 7.73-7.90 (m, 5H), 8.02 (d, 2H), 8.22-8.30 (m, 2H), 8.53 (s, 1H), 10.59 (s, 1H), 11.88 (bs, 1H).

LC-MS (Method 1): Rₜ=0.70 min; MS (ESIpos): m/z=564 [M+H−HCl]⁺.

### Example 3

### N-alpha-{[trans-4-(Aminomethyl)cyclohexyl]carbonyl}-4-isoquinolin-4-yl-N-[4-(2H-tetrazol-5-yl)phenyl]-L-phenylalaninamide hydrochloride

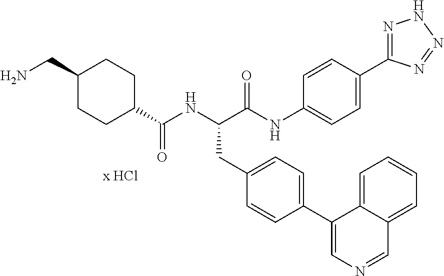

A solution of 65 mg (0.08 mmol) of tert-butyl[(trans-4-{[(2S)-3-[4-(isoquinolin-4-yl)phenyl]-1-oxo-1-{[4-(2H-tetrazol-5-yl)phenyl]amino}propan-2-yl]carbamoyl}cyclohexyl)methyl]carbamate trifluoroacetate in 2 ml of dioxane was admixed with 0.2 ml (0.83 mmol) of 4M hydrogen chloride in 1,4-dioxane and stirred at RT for 4 h. Another 0.2 ml (0.83 mmol) of 4M hydrogen chloride in 1,4-dioxane was added, and the mixture was stirred at RT for 18 h. The solid formed was filtered off, washed with acetonitrile and dried under high vacuum. 53 mg (96% of theory) of the title compound were obtained.

¹H NMR (400 MHz, DMSO-d₆): δ=ppm 0.88-1.01 (m, 2H), 1.15-1.37 (m, 2H), 1.43-1.69 (m, 2H), 1.70-1.83 (m, 3H), 2.14-2.24 (m, 1H), 2.60-2.70 (m, 2H), 3.05 (dd, 1H), 3.22 (dd, 1H), 4.77-4.85 (m, 1H), 7.50 (d, 2H), 7.57 (d, 2H), 7.70-7.96 (m, 7H), 7.97-8.06 (m, 3H), 8.35 (d, 1H), 8.47 (d, 1H), 8.54 (s, 1H), 9.70 (s, 1H), 10.62 (s, 1H).

LC-MS (Method 1): Rₜ=0.65 min; MS (ESIneg): m/z=573 [M−H−HCl]⁻.

### Example 4

### N-alpha-{[trans-4-(Aminomethyl)cyclohexyl]carbonyl}-4-(3-methyl-2-oxo-2,3-dihydro-1H-benzimidazol-5-yl)-N-[4-(2H-tetrazol-5-yl)phenyl]-L-phenylalaninamide hydrochloride

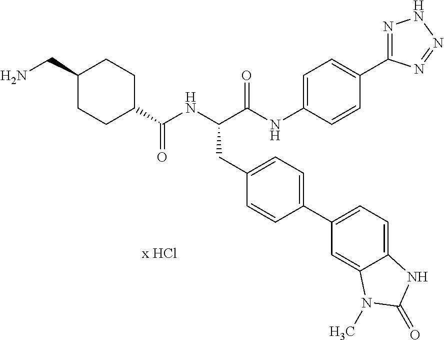

A solution of 30 mg (0.04 mmol) of N-alpha-[(trans-4-{[(tert-butoxycarbonyl)amino]methyl}-cyclohexyl)carbonyl]-4-(3-methyl-2-oxo-2,3-dihydro-1H-benzimidazol-5-yl)-N-[4-(2H-tetrazol-5-yl)phenyl]-L-phenylalaninamide in 2 ml of tetrahydrofuran was admixed with 2.00 ml (8.00 mmol) of 4M hydrogen chloride in 1,4-dioxane and stirred at RT for 16 h. The solvent was removed on a rotary evaporator, and the residue was stirred in acetonitrile and filtered. The solid formed was washed with acetonitrile and dried under high vacuum. 27 mg (90% of theory, 94% purity) of the title compound were obtained.

¹H NMR (400 MHz, DMSO-d₆): δ=ppm 0.93 (m, 2H), 1.07-1.37 (m, 2H), 1.40-1.83 (m, 5H), 2.09-2.21 (m, 1H), 2.59-2.67 (m, 2H), 2.85-3.00 (m, 1H), 3.02-3.18 (m, 1H), 3.30 (s, 3H), 4.61-4.78 (m, 1H), 7.10-7.22 (m, 2H), 7.26-7.45 (m, 3H), 7.54 (d, 2H), 7.85 (d, 5H), 8.05 (d, 1H), 8.28 (d, 1H), 10.61 (s, 1H), 10.96 (s, 1H).

LC-MS (Method 1): Rₜ=0.68 min; MS (ESIneg): m/z=592 [M−H−HCl]⁻.

### Example 5

### N-alpha-{[trans-4-(Aminomethyl)cyclohexyl]carbonyl}-4-(1-methyl-2,4-dioxo-1,2,3,4-tetrahydropyrimidin-5-yl)-N-[4-(2H-tetrazol-5-yl)phenyl]-L-phenylalaninamide hydrochloride

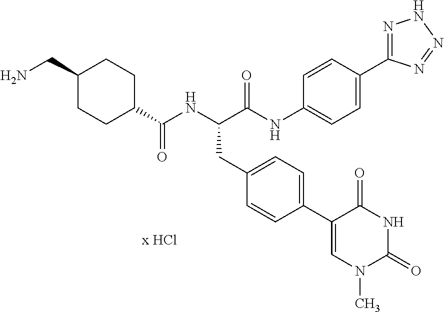

A solution of 155 mg (0.19 mmol) of N-alpha-[(trans-4-{[(tert-butoxycarbonyl)amino]methyl}-cyclohexyl)carbonyl]-4-(2,4-dimethoxypyrimidin-5-yl)-N-[4-(2H-tetrazol-5-yl)phenyl]-L-phenylalaninamide trifluoroacetate in 6 ml of 1,4-dioxane was admixed with 0.48 ml (1.93 mmol) of 4M hydrogen chloride in 1,4-dioxane and stirred at RT for 4 h. After the addition of a further 0.48 ml (1.93 mmol) of 4M hydrogen chloride in 1,4-dioxane, the reaction mixture was stirred at RT overnight. A further 0.48 ml (1.93 mmol) of the 4M hydrogen chloride in 1,4-dioxane was added, and the mixture was stirred at 50° C. overnight. The reaction mixture was concentrated on a rotary evaporator and the solid was stirred with a little methanol The precipitated solid was filtered off with suction, washed with methanol and then dried under high vacuum. This gave 77 mg (57% of theory) of the title compound.

¹H NMR (400 MHz, DMSO-d₆): δ=ppm 0.85-1.00 (m, 2H), 1.14-1.32 (m, 2H), 1.41-1.66 (m, 2H), 1.70-1.82 (m, 3H), 2.10-2.20 (m, 1H), 2.59-2.69 (m, 2H), 2.89 (dd, 1H), 3.07 (dd, 1H), 3.30 (s, 3H), 4.64-4.71 (m, 1H), 7.30 (d, 2H), 7.48 (d, 2H), 7.70-7.86 (m, 4H), 7.92 (s, 1H), 8.02 (d, 2H), 8.22 (d, 1H), 10.56 (s, 1H), 11.42 (s, 1H), 16.7 (bs, 1H).

LC-MS (Method 1): Rₜ=0.58 min; MS (ESIneg): m/z=570 [M−H−HCl]⁻.

### Example 6

### N-alpha-{[trans-4-(Aminomethyl)cyclohexyl]carbonyl}-4-(1H-indazol-4-yl)-N-[4-(2H-tetrazol-5-yl)phenyl]-L-phenylalaninamide hydrochloride

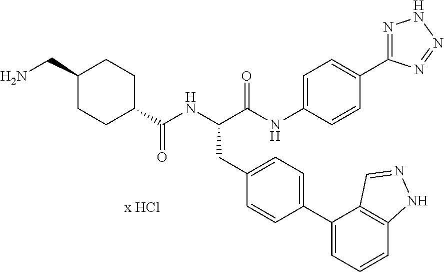 [SMILES: NC[C@H]1CC[C@H](C(=O)N[C@@H](Cc2ccc(-c3cccc4[nH]ncc34)cc2)C(=O)Nc2ccc(-c3nn[nH]n3)cc2)CC1 ; "x HCl"]

A solution of 50 mg (0.07 mmol) of N-alpha-[(trans-4-{[(tert-butoxycarbonyl)amino]methyl}-cyclohexyl)carbonyl]-4-(1H-indazol-4-yl)-N-[4-(2H-tetrazol-5-yl)phenyl]-L-phenylalaninamide in 2 ml of tetrahydrofuran was admixed with 0.5 ml (2 mmol) of 4M hydrogen chloride in 1,4-dioxane and stirred at RT for 16 h. A further 0.2 ml (1 mmol) of 4M hydrogen chloride in 1,4-dioxane was added, and the mixture was stirred at RT for 16 h. The solid formed was filtered off and washed with tetrahydrofuran, acetonitrile, ethyl acetate and dichloromethane and dried under high vacuum. 32.5 mg (69% of theory, 92% purity) of the title compound were obtained.

¹H NMR (400 MHz, DMSO-d₆): δ=ppm 0.93 (m, 2H), 1.10-1.38 (m, 2H), 1.49 (m, 1H), 1.57-1.86 (m, 4H), 2.09-2.23 (m, 1H), 2.58-2.72 (m, 2H), 2.89-3.06 (m, 1H), 3.16 (m, 1H), 4.76 (m, 1H), 7.20 (d, 1H), 7.33-7.69 (m, 7H), 7.72-7.91 (m, 5H), 7.94-8.19 (m, 3H), 8.32 (d, 1H), 10.60 (s, 1H), 13.22 (br. s, 1H).

LC-MS (Method 1): Rₜ=0.70 min; MS (ESIneg): m/z=562 [M−H−HCl]⁻.

### Example 7

### N-alpha-{[trans-4-(Aminomethyl)cyclohexyl]carbonyl}-4-(2-methyl-1H-benzimidazol-5-yl)-N-[4-(2H-tetrazol-5-yl)phenyl]-L-phenylalaninamide hydrochloride

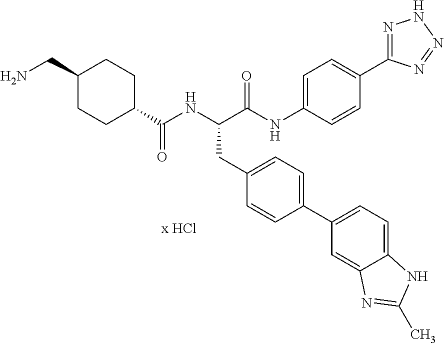 [SMILES: Cc1nc2cc(-c3ccc(C[C@H](NC(=O)[C@H]4CC[C@H](CN)CC4)C(=O)Nc4ccc(-c5nn[nH]n5)cc4)cc3)ccc2[nH]1 ; "x HCl"]

N-alpha-[(trans-4-{[(tert-Butoxycarbonyl)amino]methyl}cyclohexyl)carbonyl]-4-(2-methyl-1H-benzimidazol-5-yl)-N-[4-(2H-tetrazol-5-yl)phenyl]-L-phenylalaninamide (23 mg, 0.03 mmol) was dissolved in dichloromethane (2 ml), 4M hydrogen chloride in dioxane (0.17 mmol, 0.04 ml) was added and the mixture was stirred at 35° C. for 16 h. The reaction solution was admixed with acetonitrile, and the precipitated product was filtered off with suction, dried and purified by means of preparative HPLC (Method 7). This gave 8 mg (40% of theory) of the title compound.

¹H NMR (300 MHz, DMSO-d₆): δ=ppm 0.81-1.02 (m, 2H), 1.09-1.36 (m, 2H), 1.45 (br. s., 1H), 1.62 (d, 1H), 1.67-1.84 (m, 3H), 2.15 (t, 1H), 2.63 (d, 2H), 2.84-2.98 (m, 2H), 3.05-3.14 (m, 2H), 4.64-4.77 (m, 1H), 7.32-7.42 (m, 3H), 7.45-7.51 (m, 1H), 7.54-7.69 (m, 5H), 7.90 (d, 2H), 8.07-8.19 (m, 2H), 10.13 (s, 1H).

LC-MS (Method 4): Rₜ=0.58 min; MS (ESIneg): m/z=577 [M−H−HCl]⁻.

### Example 8

### N-alpha-{[trans-4-(Aminomethyl)cyclohexyl]carbonyl}-4-(5-methyl-1H-benzimidazol-6-yl)-N-(3-oxo-2,3-dihydro-1H-indazol-6-yl)-L-phenylalaninamide hydrochloride

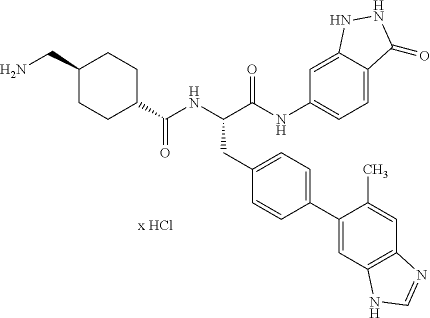

N-alpha-[(trans-4-{[(tert-Butoxycarbonyl)amino]methyl}cyclohexyl)carbonyl]-4-(5-methyl-1H-benzimidazol-6-yl)-N-(3-oxo-2,3-dihydro-1H-indazol-6-yl)-L-phenylalaninamide (20.1 mg, 0.03 mmol) was dissolved in dichloromethane (1 ml), 4M hydrogen chloride in dioxane (0.31 mmol, 0.08 ml) was added and the mixture was stirred at 40° C. for 16 h. The suspension was admixed with acetonitrile, and the solid was filtered off with suction, washed with acetonitrile and dried. This gave 11 mg (53% of theory) of the title compound.

¹H NMR (400 MHz, DMSO-d₆): δ=ppm 0.82-1.00 (m, 2H), 1.09-1.35 (m, 2H), 1.39-1.60 (m, 2H), 1.66-1.81 (m, 3H), 2.14 (m, 1H), 2.29 (s, 3H), 2.62 (t, 2H), 2.93 (m, 1H), 3.11 (dd, 1H), 4.71 (td, 1H), 6.83 (d, 1H), 7.03 (dd, 1H), 7.27 (d, 2H), 7.39 (d, 2H), 7.43 (d, 1H), 7.53 (s, 1H), 7.72 (s, 1H), 7.81 (br. s., 3H), 8.17 (d, 1H), 9.43 (s, 1H), 10.00 (s, 1H), 10.48 (s, 1H), 10.54 (s, 1H).

LC-MS (Method 4): Rₜ=0.59 min; MS (ESIneg): m/z=565 [M−H−HCl]⁻.

### Example 9

### N-alpha-{[trans-4-(Aminomethyl)cyclohexyl]carbonyl}-4-(2-oxo-1,2,3,4-tetrahydroquinolin-7-yl)-N-[4-(2H-tetrazol-5-yl)phenyl]-L-phenylalaninamide hydrochloride

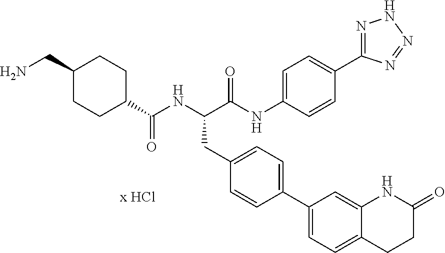

tert-Butyl[(trans-4-{[(2S)-1-oxo-3-[4-(2-oxo-1,2,3,4-tetrahydroquinolin-7-yl)phenyl]-1-{[4-(2H-tetrazol-5-yl)phenyl]amino}propan-2-yl]carbamoyl}cyclohexyl)methyl]carbamate (59 mg, 0.09 mmol) was dissolved in dichloromethane (5.2 ml), 4M hydrogen chloride in dioxane (0.86 mmol, 0.21 ml) was added and the mixture was stirred at RT for 16 h. The reaction mixture was admixed with acetonitrile and the precipitate formed was filtered off with suction and dried. Purification was effected by means of preparative HPLC (Method 7). This gave 24.2 mg (44% of theory) of the title compound.

¹H NMR (300 MHz, DMSO-d₆): δ=ppm 0.81-1.02 (m, 2H), 1.09-1.31 (m, 2H), 1.36-1.51 (m, 1H), 1.53-1.63 (m, 1H), 1.65-1.80 (m, 3H), 2.07-2.20 (m, 1H), 2.63 (d, 2H), 2.84-2.92 (m, 3H), 3.01-3.18 (m, 2H), 4.56-4.85 (m, 1H), 7.06-7.11 (m, 1H), 7.13-7.26 (m, 2H), 7.37 (d, 2H), 7.47 (d, 2H), 7.60 (d, 2H), 7.89 (d, 2H), 8.10-8.19 (m, 1H), 10.10 (s, 1H), 10.15 (s, 1H).

LC-MS (Method 4): Rₜ=0.79 min; MS (ESIneg): m/z=592 [M−H−HCl]⁻.

### Example 10

### 4-(3-Amino-1H-indazol-4-yl)-N-alpha-{[trans-4-(aminomethyl)cyclohexyl]carbonyl}-N-[4-(2H-tetrazol-5-yl)phenyl]-L-phenylalaninamide hydrochloride

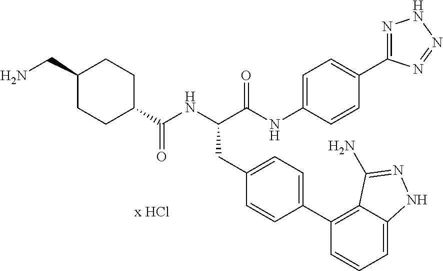 [SMILES: NC[C@H]1CC[C@H](C(=O)N[C@@H](Cc2ccc(-c3cccc4[nH]nc(N)c34)cc2)C(=O)Nc2ccc(-c3nn[nH]n3)cc2)CC1 ; "x HCl"]

4-(3-Amino-1H-indazol-4-yl)-N-alpha-[(trans-4-{[(tert-butoxycarbonyl)amino]methyl}-cyclohexyl)carbonyl]-N-[4-(2H-tetrazol-5-yl)phenyl]-L-phenylalaninamide (16.7 mg, 0.0246 mmol) was dissolved in dichloromethane (1.5 ml), 4M hydrogen chloride in dioxane (0.25 mmol, 0.06 ml) was added and the mixture was stirred at RT for 16 h. The reaction mixture was concentrated by rotary evaporation and purified by means of preparative HPLC (Method 8). This gave 5 mg (33% of theory) of the title compound.

¹H NMR (300 MHz, DMSO-d₆): δ=ppm 0.91 (d, 2H), 1.09-1.36 (m, 2H), 1.45 (m, 1H), 1.60 (m, 1H), 1.74 (br. s., 3H), 2.15 (m, 1H), 2.63 (d, 2H), 2.88-3.01 (m, 1H), 4.24 (br. s., 2H), 4.69-4.83 (m, 1H), 6.75 (t, 1H), 7.26 (d, 2H), 7.34-7.39 (d, 2H), 7.40-7.45 (d, 2H), 7.60 (d, 2H), 7.90 (d, 2H), 8.08-8.24 (m, 2H), 10.13 (s, 1H), 11.71 (br. s., 1H).

LC-MS (Method 4): Rₜ=0.76 min; MS (ESIneg): m/z=578 [M−H−HCl]⁻.

### Example 11

### N-alpha-{[trans-4-(Aminomethyl)cyclohexyl]carbonyl}-4-(1,3-dimethyl-2,4-dioxo-1,2,3,4-tetrahydropyrimidin-5-yl)-N-[4-(2H-tetrazol-5-yl)phenyl]-L-phenylalaninamide hydrochloride

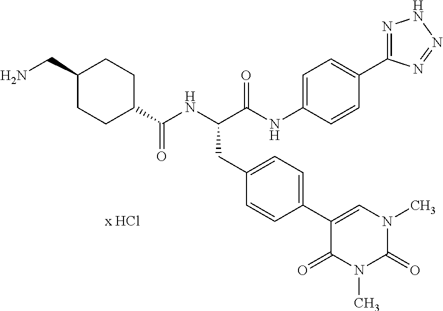

N-alpha-[(trans-4-{[(tert-Butoxycarbonyl)amino]methyl}cyclohexyl)carbonyl]-4-(1,3-dimethyl-2,4-dioxo-1,2,3,4-tetrahydropyrimidin-5-yl)-N-[4-(2H-tetrazol-5-yl)phenyl]-L-phenylalaninamide (14 mg, 0.02 mmol) was dissolved in dioxane (0.9 ml), 4M hydrogen chloride in dioxane (0.20 mmol, 0.05 ml) was added and the mixture was stirred at 40° C. for 4 hours. The reaction solution was admixed with acetonitrile, and the precipitated product was filtered off with suction and dried. This gave 8.7 mg (65% of theory) of the title compound.

¹H NMR (300 MHz, DMSO-d₆): δ=ppm 0.79-0.99 (m, 2H), 1.09-1.29 (m, 2H), 1.46 (m, 1H), 1.59 (d, 1H), 1.70 (m, 2H), 2.04-2.18 (m, 1H), 2.55-2.65 (m, 2H), 2.80-2.94 (m, 1H), 3.05 (dd, 1H), 3.20 (s, 3H), 3.35 (s, 3H), 4.59-4.72 (m, 1H), 7.28 (d, 2H), 7.47 (d, 2H), 7.67-7.83 (m, 5H), 7.92 (s, 1H), 7.98 (d, 2H), 8.16 (d, 1H), 10.48 (s, 1H).

LC-MS (Method 4): Rₜ=0.67 min; MS (ESIneg): m/z=585 [M−H−HCl]⁻.

### Example 12

### N-alpha-{[trans-4-(Aminomethyl)cyclohexyl]carbonyl}-N-[3-fluoro-4-(2H-tetrazol-5-yl)phenyl]-4-(2-oxo-1,2,3,4-tetrahydroquinolin-7-yl)-L-phenylalaninamide hydrochloride

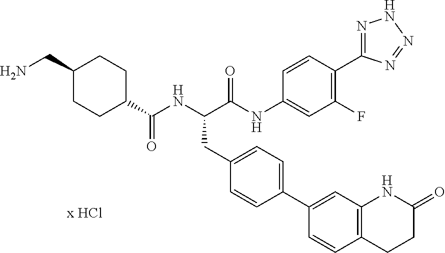

tert-Butyl[trans-4-({(2S)-1-{[3-fluoro-4-(2H-tetrazol-5-yl)phenyl]amino}-1-oxo-3-[4-(2-oxo-1,2,3,4-tetrahydroquinolin-7-yl)phenyl]propan-2-yl}carbamoyl)cyclohexyl]methyl}carbamate (51.7 mg, 0.07 mmol) was dissolved in dichloromethane (2 ml), 4M hydrogen chloride in dioxane (0.36 mmol, 0.09 ml) was added and the mixture was stirred at RT for 16 h. The reaction solution was admixed with acetonitrile, and the precipitated product was filtered off with suction and dried. This gave 38 mg (79% of theory) of the title compound.

¹H NMR (300 MHz, DMSO-d₆): δ=ppm 0.83-1.00 (m, 2H), 1.10-1.34 (m, 2H), 1.42-1.49 (m, 1H), 1.60 (m, 1H), 1.68-1.82 (m, 3H), 2.06 (s, 2H), 2.14 (t, 1H), 2.62 (m, 2H), 2.85-2.98 (m, 3H), 3.11 (dd, 1H), 4.64-4.74 (m, 1H), 7.10 (s, 1H), 7.13-7.18 (m, 1H), 7.20-7.25 (m, 1H), 7.38 (d, 2H), 7.47 (d, 2H), 7.53 (dd, 1H), 7.74 (br. s., 3H), 7.86 (dd, 1H), 8.02 (t, 1H), 8.24 (d, 1H), 10.08 (s, 1H), 10.75 (s, 1H).

LC-MS (Method 4): Rₜ=0.78 min; MS (ESIneg): m/z=610 [M−H−HCl]⁻.

### Example 13

### 4-[1-(2-Aminoethyl)-1H-benzimidazol-5-yl]-N-alpha-{[trans-4-(aminomethyl)cyclohexyl]-carbonyl]-N-[4-(2H-tetrazol-5-yl)phenyl]-L-phenylalaninamide hydrochloride

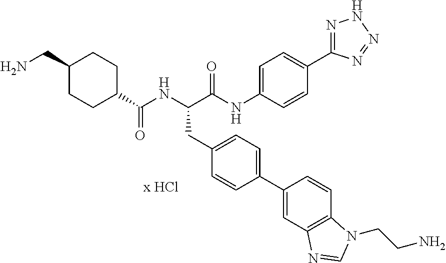

4-(1-{2-[(tert-Butoxycarbonyl)amino]ethyl -1H-benzimidazol-5-yl)-N-alpha-[(trans-4-{[(tert-butoxycarbonyl)amino]methyl}cyclohexyl)carbonyl]-N-[4-(2H-tetrazol-5-yl)phenyl]-L-phenylalaninamide (24.9 mg, 0.031 mmol) was dissolved in dichloromethane (1.9 ml), 4M hydrogen chloride in dioxane (0.31 mmol, 0.08 ml) was added and the mixture was stirred at RT for 16 h. The reaction solution was admixed with acetonitrile, and the precipitated product was filtered off with suction and dried. This gave 17 mg (82% of theory) of the title compound.

¹H NMR (300 MHz, DMSO-d₆): δ=ppm 0.91 (m, 2H), 1.12-1.32 (m, 2H), 1.48 (m, 1H), 1.61 (m, 1H), 1.74 (m, 3H), 2.16 (t, 1H), 2.61 (m, 2H), 2.90-3.01 (m, 1H), 3.08-3.19 (m, 1H), 3.33-3.60 (m, 2H), 4.76 (m, 3H), 7.45 (d, 2H), 7.68 (d, 2H), 7.84 (d, 5H), 7.99-8.11 (m, 4H), 8.24-8.50 (m, 4H), 9.33 (br. s., 1H), 10.63 (s, 1H).

LC-MS (Method 4): Rₜ=0.53 min; MS (ESIneg): m/z=606 [M−H−HCl]⁻.

### Example 14

### N-alpha-{[trans-4-(Aminomethyl)cyclohexyl]carbonyl}-4-(2,4-dioxo-1,2,3,4-tetrahydropyrimidin-5-yl)-N-[4-(2H-tetrazol-5-yl)phenyl]-L-phenylalaninamide hydrochloride

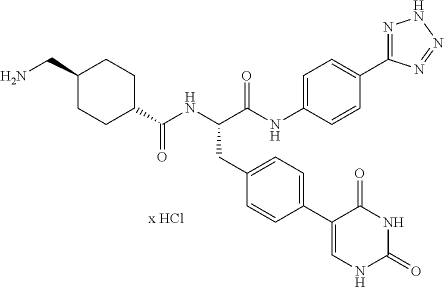

N-alpha-[(trans-4-{[(tert-Butoxycarbonyl)amino]methyl}cyclohexyl)carbonyl]-4-(2,4-dioxo-1,2,3,4-tetrahydropyrimidin-5-yl)-N-[4-(2H-tetrazol-5-yl)phenyl]-L-phenylalaninamide (30 mg, 0.05 mmol) was dissolved in dichloromethane (1 ml), 4M hydrogen chloride in dioxane (0.46 mmol, 0.11 ml) was added and the mixture was stirred at RT for 3 d. The reaction solution was admixed with acetonitrile, and the precipitated product was filtered off with suction, dried and purified by means of preparative HPLC (Method 7). This gave 5.8 mg (21% of theory) of the title compound.

¹H NMR (500 MHz, DMSO-d₆): δ=ppm 0.87-1.00 (m, 2H), 1.18-1.33 (m, 2H), 1.47 (m, 1H), 1.63 (m, 1H), 1.71-1.83 (m, 2H), 2.16 (t, 1H), 2.37 (m, 1H), 2.62-2.67 (m, 3H), 2.88 (dd, 1H), 3.07 (dd, 1H), 4.68 (td, 1H), 7.30 (d, 2H), 7.48 (d, 2H), 7.58 (s, 1H), 7.62 (d, 2H), 7.91 (d, 2H), 8.11 (d, 1H), 8.15 (s, 1H), 10.13 (s, 1H), 11.21 (br. s., 1H).

LC-MS (Method 4): Rₜ=0.57 min; MS (ESIneg): m/z=557 [M−H−HCl]⁻.

### Example 15

### N-alpha-{[trans-4-(Aminomethyl)cyclohexyl]carbonyl}-4-(1-methyl-1H-benzimidazol-6-yl)-N-[4-(2H-tetrazol-5-yl)phenyl]-L-phenylalaninamide hydrochloride

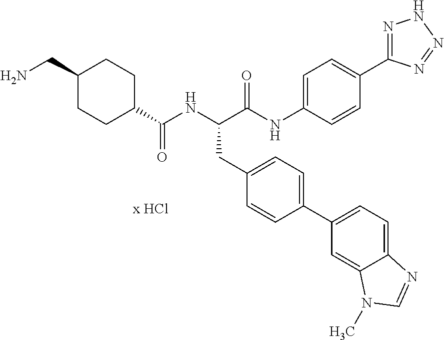

N-alpha-[(trans-4-{[(tert-Butoxycarbonyl)amino]methyl}cyclohexyl)carbonyl]-4-(1-methyl-1H-benzimidazol-6-yl)-N-[4-(2H-tetrazol-5-yl)phenyl]-L-phenylalaninamide (27 mg, 0.04 mmol) was dissolved in dichloromethane (2.4 ml), 4M hydrogen chloride in dioxane (0.40 mmol, 0.10 ml) was added and the mixture was stirred at 35° C. for 16 h. The reaction solution was purified by means of preparative HPLC (Method 7). This gave 7 mg (27% of theory) of the title compound.

¹H NMR (300 MHz, DMSO-d₆): δ=ppm 0.81-1.02 (m, 2H), 1.15-1.35 (m, 2H), 1.44 (m, 1H), 1.57-1.84 (m, 4H), 2.16 (m, 1H), 2.63 (d, 2H), 2.85-3.00 (m, 1H), 3.11 (dd, 1H), 3.87 (s, 3H), 4.61-4.79 (m, 1H), 7.40 (d, 2H), 7.49 (dd, 1H), 7.60 (d, 2H), 7.64-7.71 (m, 3H), 7.82 (d, 1H), 7.90 (d, 2H), 8.09-8.22 (m, 2H), 10.12 (s, 1H).

LC-MS (Method 4): Rₜ=0.69 min; MS (ESIneg): m/z=577 [M−H−HCl]⁻.

### Example 16

### N-alpha-{[trans-4-(Aminomethyl)cyclohexyl]carbonyl}-4-(1-methyl-1H-benzimidazol-5-yl)-N-[4-(2H-tetrazol-5-yl)phenyl]-L-phenylalaninamide hydrochloride

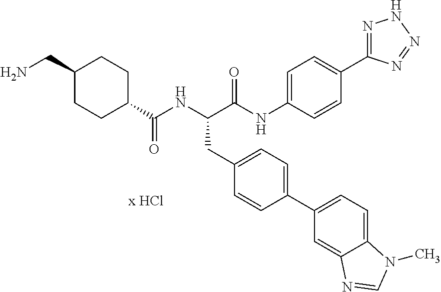 [SMILES: Cn1cnc2cc(-c3ccc(C[C@H](NC(=O)[C@H]4CC[C@H](CN)CC4)C(=O)Nc4ccc(-c5nn[nH]n5)cc4)cc3)ccc21]

N-alpha-[(trans-4-{[(tert-Butoxycarbonyl)amino]methyl}cyclohexyl)carbonyl]-4-(1-methyl-1H-benzimidazol-5-yl)-N-[4-(2H-tetrazol-5-yl)phenyl]-L-phenylalaninamide (11 mg, 0.017 mmol) was dissolved in dichloromethane (1 ml), 4M hydrogen chloride in dioxane (0.08 mmol, 0.02 ml) was added and the mixture was stirred at 35° C. for 16 h. The reaction solution was admixed with acetonitrile, and the precipitated product was filtered off with suction, dried and purified by means of preparative HPLC (Method 7). This gave 2 mg (20% of theory) of the title compound.

¹H NMR (300 MHz, DMSO-d₆): δ=ppm 0.82-1.01 (m, 3H), 1.12-1.35 (m, 3H), 1.45 (m, 1H), 1.62 (m, 1H), 1.77 (m, 3H), 2.15 (m, 1H), 2.63 (d, 1H), 2.91 (dd, 1H), 3.85 (s, 3H), 4.70 (m, 1H), 7.38 (d, 2H), 7.56-7.66 (m, 7H), 7.85-7.94 (m, 3H), 8.09-8.16 (m, 1H), 8.19 (s, 1H), 10.13 (s, 1H).

LC-MS (Method 4): Rₜ=0.62 min; MS (ESIneg): m/z=577 [M−H−HCl]⁻.

### Example 17

### N-alpha-{[trans-4-(Aminomethyl)cyclohexyl]carbonyl}-4-(1-benzyl-2,4-dioxo-1,2,3,4-tetrahydropyrimidin-5-yl)-N-[4-(2H-tetrazol-5-yl)phenyl]-L-phenylalaninamide hydrochloride

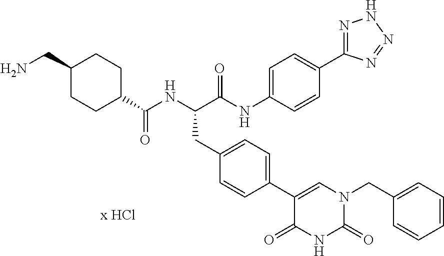

4-(1-Benzyl-2,4-dioxo-1,2,3,4-tetrahydropyrimidin-5-yl)-N-alpha-[(trans-4-{[(tert-butoxy-carbonyl)amino]methyl}cyclohexyl)carbonyl]-N-[4-(2H-tetrazol-5-yl)phenyl]-L-phenylalaninamide (25 mg, 0.034 mmol) was dissolved in dichloromethane (1 ml), 4M hydrogen chloride in dioxane (0.3 mmol, 0.08 ml) was added and the mixture was stirred at 35° C. for 16 h. The reaction solution was purified by preparative HPLC (Method 7). This gave 8 mg (33% of theory) of the title compound.

¹H NMR (300 MHz, DMSO-d₆): δ=ppm 0.76-0.99 (m, 2H), 1.08-1.28 (m, 2H), 1.34-1.49 (m, 1H), 1.51-1.63 (m, 1H), 1.65-1.78 (m, 3H), 2.04-2.18 (m, 1H), 2.55-2.64 (m, 2H), 2.79-2.93 (m, 1H), 2.98-3.09 (m, 1H), 4.58-4.70 (m, 1H), 4.88-4.96 (m, 2H), 7.19-7.35 (m, 7H), 7.45 (d, 2H), 7.63 (br. s, 3H), 7.77 (d, 2H), 7.95 (d, 2H), 8.03 (s, 1H), 8.12 (d, 1H), 10.43 (s, 1H), 11.46 (s, 1H).

LC-MS (Method 4): Rₜ=0.79 min; MS (ESIneg): m/z=647.2 [M−H−HCl]⁻.

### Example 18

### N-alpha-{[trans-4-(Aminomethyl)cyclohexyl]carbonyl}-4-(5-methyl-1H-indazol-4-yl)-N-[4-(2H-tetrazol-5-yl)phenyl]-L-phenylalaninamide hydrochloride

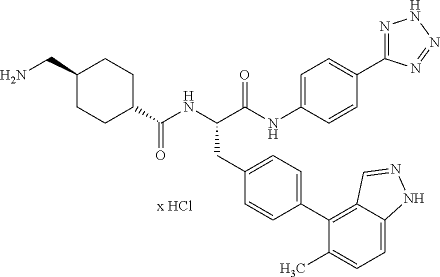

N-alpha-[(trans-4-{[(tert-Butoxycarbonyl)amino]methyl}cyclohexyl)carbonyl]-4-(5-methyl-1H-indazol-4-yl)-N-[4-(2H-tetrazol-5-yl)phenyl]-L-phenylalaninamide (20 mg, 0.03 mmol) was dissolved in dioxane (1 ml), 4M hydrogen chloride in dioxane (0.3 mmol, 0.08 ml) was added and the mixture was stirred at 30° C. for 3 days. The reaction solution was admixed with acetonitrile, and the precipitated product was filtered off with suction and dried. This gave 14 mg (67% of theory) of the title compound.

¹H NMR (400 MHz, DMSO-d₆): δ=ppm 0.80-0.97 (m, 2H), 1.09-1.32 (m, 2H), 1.37-1.49 (m, 1H), 1.54-1.62 (m, 1H), 1.64-1.78 (m, 4H), 2.09-2.16 (m, 1H), 2.18 (s, 3H), 2.56-2.63 (m, 2H), 2.90-3.00 (m, 1H), 3.08-3.17 (m, 1H), 4.71-4.81 (m, 1H), 7.20-7.30 (m, 3H), 7.36-7.46 (m, 4H), 7.68 (br. s, 3H), 7.80 (d, 2H), 7.97 (d, 2H), 8.23 (d, 1H), 10.45 (s, 1H).

LC-MS (Method 4): Rₜ=0.83 min; MS (ESIneg): m/z=577.2 [M−H−HCl]⁻.

### Example 19

### N-alpha-{[trans-4-(Aminomethyl)cyclohexyl]carbonyl}-4-(2-isopropyl-6-methyl-1H-benzimidazol-5-yl)-N-(2-oxo-2,3-dihydro-1H-benzimidazol-5-yl)-L-phenylalaninamide

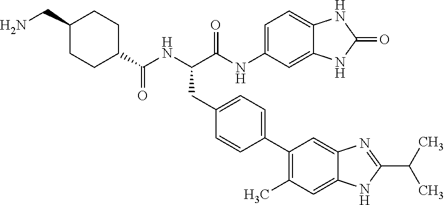

A solution of 390 mg (0.55 mmol) of N-alpha-[(trans-4-{[(tert-butoxycarbonyl)amino]methyl}-cyclohexyl)carbonyl]-4-(2-isopropyl-6-methyl-1H-benzimidazol-5-yl)-N-(2-oxo-2,3-dihydro-1H-benzimidazol-5-yl)-L-phenylalaninamide in 11 ml of dichloromethane was admixed with 0.7 ml (2.75 mmol) of 4M hydrogen chloride in 1,4-dioxane and stirred at 35° C. for 1 h, and admixed with a further 0.14 ml (0.55 mmol) of 4M hydrogen chloride in 1,4-dioxane. After stirring at 35° C. for 1 h, the reaction mixture was admixed with acetonitrile, and the precipitate was filtered off, washed with acetonitrile and dried under high vacuum. The residue was suspended in 2M aqueous sodium hydrogencarbonate solution and the mixture was stirred for 15 min Subsequently, the residue was filtered off with suction, washed with water and dried via freeze-drying. This gave 205 mg (61% of theory) of the title compound.

¹1-1NMR (400 MHz, DMSO-d₆): 6=0.70-0.88 (m, 2H), 1.02-1.29 (m, 3H), 1.32 (d, 6H), 1.48-1.60 (m, 1H), 1.70 (m, 3H), 2.04-2.16 (m, 1H), 2.20 (s, 3H), 2.32 (d, 2H), 2.83-2.96 (m, 1H), 3.02-3.16 (m, 2H), 4.57-4.79 (m, 1H), 6.83 (d, 1H), 6.97-7.05 (m, 1H), 7.21 (d, 2H), 7.27-7.37 (m, 3H), 7.44 (d, 1H), 8.08 (d, 1H), 9.96 (s, 1H).

LC-MS (Method 4): Rₜ=0.97 min; MS (ESIpos): m/z=708.5 [M+H]⁺.

### Example 20

### N-alpha-{[trans-4-(Aminomethyl)cyclohexyl]carbonyl}-N-(7-chloro-2-oxo-2,3-dihydro-1H-benzimidazol-5-yl)-4-(2-cyclopropyl-6-methyl-1H-benzimidazol-5-yl)-L-phenylalaninamide

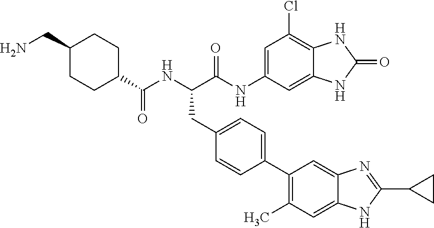

A solution of 29 mg (0.04 mmol) of N-alpha-[(trans-4-{[(tert-butoxycarbonyl)amino]-methyl}cyclohexyl)carbonyl]-N-(7-chloro-2-oxo-2,3-dihydro-1H-benzimidazol-5-yl)-4-(2-cyclopropyl-6-methyl-1H-benzimidazol-5-yl)-L-phenylalaninamide in 1 ml of dichloromethane was admixed with 0.05 ml (0 2 mmol) of 4M hydrogen chloride in 1,4-dioxane and stirred at 35° C. for 1 h and at RT overnight. The reaction mixture was then admixed with acetonitrile, and the precipitate was filtered off, washed with acetonitrile and dried under high vacuum. The residue was purified by chromatography via HPLC (Method 11). This gave 6 mg (24% of theory) of the title compound.

¹1-1NMR (400 MHz, DMSO-d₆): δ=ppm 0.75-0.89 (m, 2H), 0.97-1.07 (m, 4H), 1.09-1.20 (m, 2H), 1.20-1.32 (m, 2H), 1.51-1.61 (m, 1H), 1.66-1.80 (m, 3H), 2.03-2.14 (m, 2H), 2.20 (s, 3H), 2.31-2.39 (m, 2H), 2.91 (dd, 1H), 3.07 (dd, 1H), 4.61-4.71 (m, 1H), 7.06-7.18 (m, 1H), 7.19-7.24 (m, 3H), 7.26-7.29 (m, 1H), 7.29-7.35 (m, 3H), 8.07-8.17 (m, 1H), 10.01-10.11 (m, 1H).

LC-MS (Method 4): Rₜ=0.62 min; MS (ESIneg): m/z=640.1 [M−H]⁻.

### Example 21

### N-alpha-{[trans-4-(Aminomethyl)cyclohexyl]carbonyl}-4-(2-cyclopropyl-5-methyl-1H-imidazo[4,5-b]pyridin-6-yl)-N-(2-oxo-2,3-dihydro-1H-benzimidazol-5-yl)-L-phenylalaninamide

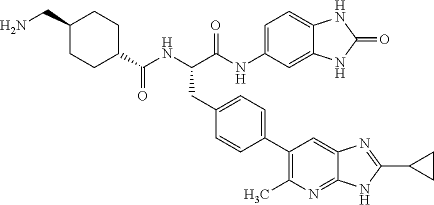

A suspension of 471 mg (0.66 mmol) of N-alpha-[(trans-4-{[(tert-butoxycarbonyl)amino]-methyl}cyclohexyl)carbonyl]-4-(2-cyclopropyl-5-methyl-1H-imidazo[4,5-b]pyridin-6-yl)-N-(2-oxo-2,3-dihydro-1H-benzimidazol-5-yl)-L-phenylalaninamide in 56 ml of dichloromethane was admixed with 1.2 ml (4.66 mmol) of 4M hydrogen chloride in 1,4-dioxane and stirred at RT overnight. After addition of a further 0.33 ml (1.33 mmol) of 4M hydrogen chloride in 1,4-dioxane and stirring overnight, acetonitrile was added, and the precipitate was filtered off, washed with acetonitrile and dried under high vacuum. The residue was purified by chromatography via HPLC (Method 11). This gave 14 mg (3% of theory) of the title compound.

¹H NMR (400 MHz, DMSO-d₆): δ=ppm 0.75-0.90 (m, 2H), 1.01-1.13 (m, 5H), 1.14-1.19 (m, 1H), 1.20-1.31 (m, 2H), 1.51-1.60 (m, 1H), 1.65-1.81 (m, 3H), 2.07-2.17 (m, 2H), 2.37 (s, 3H), 2.91 (dd, 1H), 3.09 (dd, 1H), 4.66-4.75 (m, 1H), 6.84 (d, 1H), 7.00-7.05 (m, 1H), 7.25-7.31 (m, 2H), 7.33-7.39 (m, 2H), 7.43-7.46 (m, 1H), 7.48-7.54 (m, 1H), 8.07-8.13 (m, 1H), 9.94-9.99 (m, 1H).

LC-MS (Method 4): Rₜ=0.58 min; MS (ESIpos): m/z=607.4 [M+H]⁺.

### Example 22

### N-alpha-{[trans-4-(Aminomethyl)cyclohexyl]carbonyl}-4-(2-cyclopropyl-6-methyl-1H-benzimidazol-5-yl)-N-(4-fluoro-3-oxo-2,3-dihydro-1H-indazol-6-yl)-L-phenylalaninamide hydrochloride

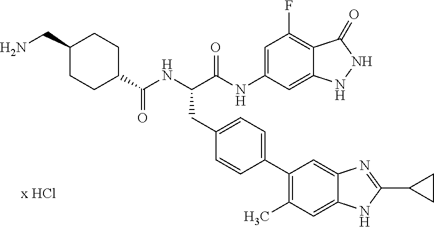

A suspension of 34 mg (0.05 mmol) of N-alpha-[(trans-4-{[(tert-butoxycarbonyl)amino]-methyl}cyclohexyl)carbonyl]-4-(2-cyclopropyl-6-methyl-1H-benzimidazol-5-yl)-N-(4-fluoro-3-oxo-2,3-dihydro-1H-indazol-6-yl)-L-phenylalaninamide in 1.2 ml of dichloromethane was admixed with 0.06 ml (0.24 mmol) of 4M hydrogen chloride in 1,4-dioxane and stirred at RT overnight. The reaction mixture was admixed with acetonitrile, and the precipitate was filtered off, washed with acetonitrile and dried under high vacuum. The residue was purified by chromatography via HPLC (Method 7). This gave 13 mg (40% of theory) of the title compound.

¹H NMR (400 MHz, DMSO-d₆): δ=ppm 0.84-1.00 (m, 2H), 1.10-1.21 (m, 1H), 1.22-1.34 (m, 1H), 1.37-1.43 (m, 4H), 1.45-1.52 (m, 1H), 1.53-1.61 (m, 1H), 1.69-1.83 (m, 4H), 2.10-2.21 (m, 1H), 2.26 (s, 3H), 2.43-2.47 (m, 1H), 2.61-2.70 (m, 2H), 2.96 (dd, 1H), 3.14 (dd, 1H), 4.68-4.79 (m, 1H), 6.82-6.90 (m, 1H), 7.24-7.30 (m, 2H), 7.35-7.44 (m, 3H), 7.57-7.63 (m, 2H), 7.76-7.90 (m, 3H), 8.25-8.33 (m, 1H), 10.31-10.45 (m, 1H).

LC-MS (Method 4): Rₜ=0.59 min; MS (ESIpos): m/z=623.9 [M+H−HCl]⁺.

### Example 23

### N-alpha-{[trans-4-(Aminomethyl)cyclohexyl]carbonyl}-4-(6-chloro-2-cyclopropyl-1H-benzimidazol-5-yl)-N-(2-oxo-2,3-dihydro-1H-benzimidazol-5-yl)-L-phenylalaninamide hydrochloride

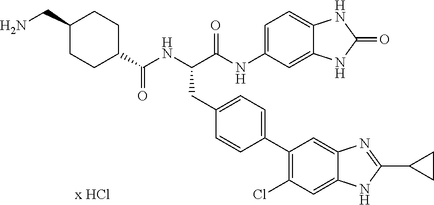

A suspension of 36 mg (50 μmol) of N-alpha-[(trans-4-{[(tert-butoxycarbonyl)amino]methyl}-cyclohexyl)carbonyl]-4-(6-chloro-2-cyclopropyl-1H-benzimidazol-5-yl)-N-(2-oxo-2,3-dihydro-1H-benzimidazol-5-yl)-L-phenylalaninamide in 4 ml of dichloromethane was admixed with 0.09 ml (0.35 mmol) of 4M hydrogen chloride in 1,4-dioxane and stirred at RT overnight. The reaction mixture was admixed with acetonitrile, and the precipitate was filtered off, washed with acetonitrile and dried under high vacuum. The residue was purified by chromatography via HPLC (Method 8). This gave 15 mg (45% of theory) of the title compound.

¹H NMR (400 MHz, DMSO-d₆): δ=ppm 0.82-1.00 (m, 2H), 1.06-1.23 (m, 1H), 1.24-1.35 (m, 4H), 1.40-1.50 (m, 1H), 1.52-1.60 (m, 1H), 1.68-1.81 (m, 3H), 2.08-2.18 (m, 1H), 2.33-2.37 (m, 1H), 2.59-2.65 (m, 2H), 2.91 (dd, 1H), 3.11 (dd, 1H), 4.66-4.76 (m, 1H), 6.83 (d, 1H), 7.00-7.05 (m, 1H), 7.30-7.35 (m, 2H), 7.36-7.40 (m, 2H), 7.41-7.44 (m, 1H), 7.45-7.50 (m, 1H), 7.70-7.83 (m, 4H), 8.11-8.19 (m, 1H), 9.97-10.04 (m, 1H), 10.44-10.51 (m, 1H), 10.51-10.59 (m, 1H).

LC-MS (Method 4): Rₜ=0.69 min; MS (ESIpos): m/z=626.3 [M+H−HCl]⁺.

### Example 24

### 3-[5-(4-{[N-{[trans-4-(Aminomethyl)cyclohexyl]carbonyl}-4-(2-isopropyl-6-methyl-1H-benzimidazol-5-yl)-L-phenylalanyl]amino}phenyl)-4H-1,2,4-triazol-3-yl]-2,2,3,3-tetrafluoropropanoic acid hydrochloride

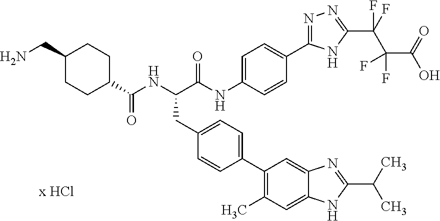

A suspension of 16 mg (18 μmol) of 3-{5-[4-({N-[(trans-4-{[(tert-butoxycarbonyl)amino]-methyl}cyclohexyl)carbonyl]-4-(2-isopropyl-6-methyl-1H-benzimidazol-5-yl)-L-phenylalany}-amino)phenyl]-4H-1,2,4-triazol-3-yl]-2,2,3,3-tetrafluoropropanoic acid in 2 ml of dichloromethane was admixed with 0.02 ml (0.09 mmol) of 4M hydrogen chloride in 1,4-dioxane and stirred at 35° C. for 2 h. The reaction mixture was admixed with acetonitrile, and the precipitate was filtered off, washed with acetonitrile and dried under high vacuum. The residue was purified by chromatography via HPLC (Method 7). This gave 2 mg (12% of theory) of the title compound.

¹H NMR (400 MHz, DMSO-d₆): δ=ppm 0.85-0.99 (m, 2H), 1.11-1.38 (m, 2H), 1.47 (d, 6H), 1.55-1.62 (m, 1H), 1.67-1.83 (m, 3H), 2.12-2.21 (m, 1H), 2.28 (s, 3H), 2.62-2.66 (m, 2H), 2.98 (dd, 1H), 3.15 (dd, 1H), 3.45-3.53 (m, 1H), 4.73-4.82 (m, 1H), 7.24-7.29 (m, 2H), 7.39-7.44 (m, 3H), 7.63-7.67 (m, 1H), 7.75-7.80 (m, 3H), 7.81-7.91 (m, 3H), 7.95-8.01 (m, 3H), 8.27-8.32 (m, 1H), 10.40-10.52 (m, 1H).

LC-MS (Method 4): Rₜ=0.67 min; MS (ESIpos): m/z=763.5 [M+H−HCl]⁺.

### Example 25

### N-alpha-{[trans-4-(Aminomethyl)cyclohexyl]carbonyl}-4-(2-isopropyl-6-methyl-1H-benzimidazol-5-yl)-N-[2-(pentafluoroethyl)-1H-benzimidazol-6-yl]-L-phenylalaninamide hydrochloride

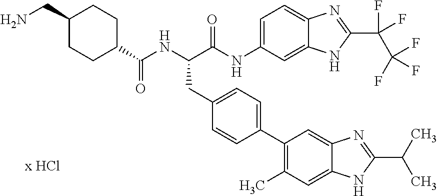

A suspension of 18 mg (22 μmol) of N-alpha-[(trans-4-{[(tert-butoxycarbonyl)amino]methyl}-cyclohexyl)carbonyl]-4-(2-isopropyl-6-methyl-1H-benzimidazol-5-yl)-N-[2-(pentafluoroethyl)-1H-benzimidazol-6-yl]-L-phenylalaninamide in 2 ml of dichloromethane was admixed with 0.03 ml (0.11 mmol) of 4M hydrogen chloride in 1,4-dioxane and stirred at 35° C. for 2 h. The reaction mixture was admixed with acetonitrile, and the precipitate was filtered off, washed with acetonitrile and dried under high vacuum. This gave 11 mg (63% of theory) of the title compound.

¹H NMR (400 MHz, DMSO-d₆): δ=ppm 0.85-1.00 (m, 2H), 1.13-1.34 (m, 2H), 1.47 (d, 6H), 1.55-1.63 (m, 1H), 1.71-1.82 (m, 3H), 2.13-2.23 (m, 1H), 2.27 (s, 3H), 2.63-2.66 (m, 2H), 2.98 (dd, 1H), 3.16 (dd, 1H), 4.75-4.83 (m, 1H), 7.24-7.30 (m, 2H), 7.38-7.43 (m, 2H), 7.44-7.47 (m, 1H), 7.63-7.66 (m, 1H), 7.73-7.79 (m, 3H), 8.21-8.24 (m, 1H), 8.24-8.29 (m, 1H), 10.31-10.40 (m, 1H).

LC-MS (Method 4): Rₜ=0.81 min; MS (ESIpos): m/z=710.4 [M+H−HCl]⁺.

### Example 26

### N-alpha-{[trans-4-(Aminomethyl)cyclohexyl]carbonyl}-4-(2-cyclopropyl-6-methyl-1,3-benzoxazol-5-yl)-N-[4-(1H-tetrazol-5-yl)phenyl]-L-phenylalaninamide hydrochloride

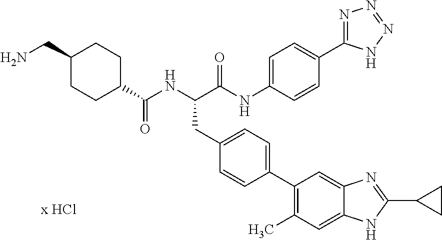

A suspension of 74 mg (0 1 mmol) of N-alpha-[(trans-4-{[(tert-butoxycarbonyl)amino]methyl}-cyclohexyl)carbonyl]-4-(2-cyclopropyl-6-methyl-1,3-benzoxazol-5-yl)-N-[4-(1H-tetrazol-5-yl)phenyl]-L-phenylalaninamide in 9 ml of dichloromethane was admixed with 0.13 ml (0 5 mmol) of 4M hydrogen chloride in 1,4-dioxane and stirred at RT overnight. The reaction mixture was admixed with acetonitrile, and the precipitate was filtered off, washed with acetonitrile and dried under high vacuum. This gave 56 mg (80% of theory) of the title compound.

¹H NMR (400 MHz, DMSO-d₆): δ=ppm 0.84-1.02 (m, 2H), 1.06-1.32 (m, 7H), 1.43-1.54 (m, 1H), 1.55-1.65 (m, 1H), 1.69-1.85 (m, 4H), 2.10-2.18 (m, 1H), 2.22 (br. s., 3H), 2.24-2.31 (m, 2H), 2.64-2.71 (m, 2H), 2.90-3.03 (m, 1H), 3.08-3.19 (m, 1H), 4.69-4.83 (m, 1H), 7.24 (d, 2H), 7.31 (br. s., 1H), 7.38 (d, 2H), 7.53 (br. s., 1H), 7.79 (br. s., 3H), 7.84 (d, 2H), 8.02 (d, 2H), 8.22-8.34 (m, 1H), 10.47-10.60 (m, 1H).

LC-MS (Method 4): Rₜ=1.02 min; MS (ESIpos): m/z=619.4 [M+H−HCl]⁺.

### Example 27

### N-alpha-{[trans-4-(Aminomethyl)cyclohexyl]carbonyl}-4-(2-cyclobutyl-7-fluoro-6-methyl-1H-benzimidazol-5-yl)-N-(2-oxo-2,3-dihydro-1H-benzimidazol-5-yl)-L-phenylalaninamide

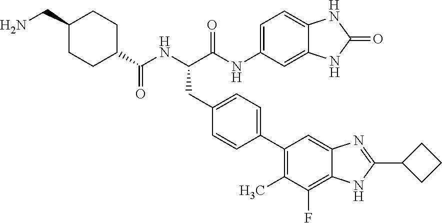

A suspension of 65 mg (0.09 mmol) of N-alpha-[(trans-4-{[(tert-butoxy carbonyl)amino]methyl}-cyclohexyl)carbonyl]-4-(2-cyclobutyl-7-fluoro-6-methyl-1H-benzimidazol-5-yl)-N-(2-oxo-2,3-dihydro-1H-benzimidazol-5-yl)-L-phenylalaninamide in 7 ml of dichloromethane was admixed with 0.16 ml (0.6 mmol) of 4M hydrogen chloride in 1,4-dioxane and stirred at RT overnight. The reaction mixture was admixed with acetonitrile, and the precipitate was filtered off, washed with acetonitrile and dried under high vacuum. The residue was purified by chromatography via HPLC (Method 11). This gave 30 mg (57% of theory) of the title compound.

¹H NMR (400 MHz, DMSO-d₆): δ=ppm 0.73-0.89 (m, 2H), 1.05-1.34 (m, 2H), 1.49-1.59 (m, 1H), 1.63-1.80 (m, 3H), 1.88-1.99 (m, 1H), 2.02-2.09 (m, 1H), 2.10-2.14 (m, 3H), 2.31-2.45 (m, 6H), 2.87-2.95 (m, 1H), 3.06-3.12 (m, 1H), 3.67-3.76 (m, 1H), 4.64-4.77 (m, 1H), 6.81-6.88 (m, 1H), 6.99-7.06 (m, 2H), 7.22-7.28 (m, 2H), 7.33-7.38 (m, 2H), 7.42-7.46 (m, 1H), 8.06-8.12 (m, 1H), 9.92-9.99 (m, 1H).

LC-MS (Method 4): Rₜ=0.74 min; MS (ESIpos): m/z=638.4 [M+H]⁺.

### Example 28

### N-alpha-{[trans-4-(Aminomethyl)cyclohexyl]carbonyl}-4-(6-methyl-1,2,3,4-tetrahydropyrido[2,3-b]pyrazin-7-yl)-N-(2-oxo-2,3-dihydro-1H-benzimidazol-5-yl)-L-phenylalaninamide hydrochloride

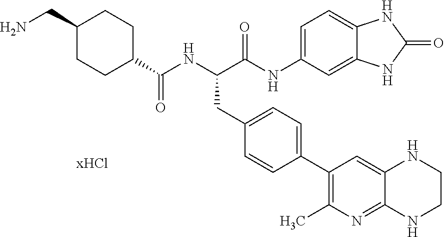

A suspension of 14 mg (0.02 mmol) of tert-butyl{[trans-4-({(2S)-3-[4-(6-methyl-1,2,3,4-tetrahydropyrido[2,3-b]pyrazin-7-yl)phenyl]-1-oxo-1-[(2-oxo-2,3-dihydro-1H-benzimidazol-5-yl)amino}propan-2-yl}carbamoyl)cyclohexyl]methyl}carbamate in 1 ml of dichloromethane was admixed with 0.02 ml (0.08 mmol) of 4M hydrogen chloride in 1,4-dioxane and stirred at RT for 48 h. The reaction mixture was admixed with acetonitrile, and the precipitate was filtered off, washed with acetonitrile and dried under high vacuum. This gave 12 mg (92% of theory) of the title compound.

¹H NMR (400 MHz, DMSO-d₆): δ=ppm 0.82-1.00 (m, 2H), 1.09-1.34 (m, 2H), 1.40-1.60 (m, 2H), 1.67-1.83 (m, 3H), 2.09-2.16 (m, 1H), 2.21 (s, 3H), 2.60-2.66 (m, 2H), 2.85-2.97 (m, 1H), 3.04-3.12 (m, 1H), 3.23-3.29 (m, 2H), 3.49-3.58 (m, 2H), 4.61-4.78 (m, 1H), 6.54-6.69 (m, 1H), 6.84 (s, 2H), 6.97-7.06 (m, 1H), 7.20-7.26 (m, 2H), 7.33-7.38 (m, 2H), 7.40-7.43 (m, 1H), 7.72-7.92 (m, 4H), 8.13-8.23 (m, 1H), 9.93-10.08 (m, 1H), 10.46-10.53 (m, 1H), 10.53-10.61 (m, 1H), 13.60-13.98 (m, 1H).

LC-MS (Method 4): Rₜ=0.65 min; MS (ESIpos): m/z=583.3 [M+H−HCl]⁺.

### Example 29

### 3-[5-(4-{[N-{[trans-4-(Aminomethyl)cyclohexyl]carbonyl}-4-(1-oxo-2,3-dihydro-1H-isoindol-5-yl)-L-phenylalanyl}amino}phenyl)-1H-1,2,4-triazol-3-yl]-2,2,3,3-tetrafluoropropanoic acid hydrochloride

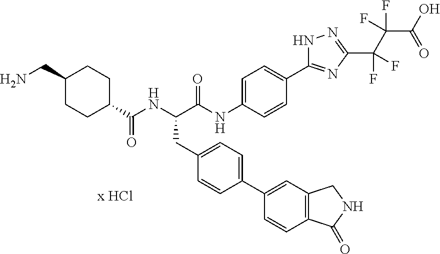 [SMILES: NC[C@H]1CC[C@H](C(=O)N[C@@H](Cc2ccc(-c3ccc4c(c3)CNC4=O)cc2)C(=O)Nc2ccc(-c3nc(C(F)(F)C(F)(F)C(=O)O)n[nH]3)cc2)CC1]

To a solution of 77 mg (93 μmol) of 3-{5-[4-({N-[(trans-4-{[(tert-butoxycarbonyl)-amino]methyl}cyclohexyl)carbonyl]-4-(1-oxo-2,3-dihydro-1H-isoindol-5-yl)-L-phenylalanyl}-amino)phenyl]-1H-1,2,4-triazol-3-yl]-2,2,3,3-tetrafluoropropanoic acid in 2 ml of dioxane were added 350 μl (1.4 mmol) of 4M hydrogen chloride in dioxane. The mixture was then stirred at RT for 18 h. Acetonitrile was added and the solid obtained was filtered, washed with acetonitrile and dried under high vacuum. 69 mg (89% of theory) of the title compound were obtained.

¹H NMR (400 MHz, DMSO-d₆): δ=ppm 0.81-1.02 (m, 2H), 1.11-1.36 (m, 2H), 1.41-1.53 (m, 1H), 1.55-1.66 (m, 1H), 1.69-1.85 (m, 3H), 2.10-2.23 (m, 1H), 2.58-2.67 (m, 2H), 2.90-3.01 (m, 1H), 3.09-3.19 (m, 1H), 4.42 (s, 2H), 4.68-4.78 (m, 1H), 7.45 (d, 2H), 7.66 (d, 2H), 7.73 (m, 2H), 7.78-7.90 (m, 5H), 8.00 (d, 2H), 8.26 (d, 1H), 8.56 (s, 1H), 10.56 (s, 1H), 14.96-15.38 (m, 1H).

LC-MS (Method 1): Rₜ=0.60 min; MS (ESIpos): m/z=722.4 [M+H−HCl]⁺.

### Example 30

### N-alpha-{[trans-4-(Aminomethyl)cyclohexyl]carbonyl}-4-(5-methyl-1H-benzimidazol-6-yl)-N-(3-oxo-2,3-dihydro-1H-indazol-6-yl)-L-phenylalaninamide hydrochloride

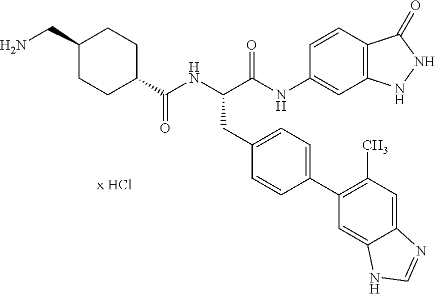 [SMILES: Cc1cc2nc[nH]c2cc1-c1ccc(C[C@H](NC(=O)[C@H]2CC[C@H](CN)CC2)C(=O)Nc2ccc3c(=O)[nH][nH]c3c2)cc1]

A solution of N-alpha-[(trans-4-{[(tert-Butoxycarbonyl)amino]methyl}cyclohexyl)carbonyl]-4-(5-methyl-1H-benzimidazol-6-yl)-N-(3-oxo-2,3-dihydro-1H-indazol-6-yl)-L-phenylalaninamide (45 mg, 0.059 mmol) in dioxane (2.5 ml) was admixed with 4M hydrogen chloride in 1,4-dioxane (0.22 ml, 0.89 mmol) and stirred at RT for 4 days. The solvent was removed on a rotary evaporator and the residue was dissolved in DMSO/acetonitrile (about 3 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 13.9 mg (36% of theory) of the title compound.

¹H NMR (400 MHz, DMSO-d₆): δ=ppm 0.93 (d, 2H), 1.10-1.34 (m, 2H), 1.46-1.61 (m, 2H), 1.76 (m, 3H), 2.16 (m, 1H), 2.30 (s, 3H), 2.60-2.69 (m, 2H), 2.92-3.03 (m, 1H), 3.10-3.21 (m, 1H), 4.78 (dd, 1H), 7.04 (d, 1H), 7.28 (d, 2H), 7.42 (d, 2H), 7.51-7.60 (m, 2H), 7.75 (s, 1H), 7.83-7.98 (m, 4H), 8.27 (d, 1H), 9.55 (s, 1H), 10.33 (br. s., 1H), 11.20 (br. s., 1H), 15.09 (br. s., 1H).

LC-MS (Method 1): Rₜ=0.45 min; MS (ESIneg): m/z=564 [M−H−HCl]⁻.

### Example 31

### N-alpha-{[trans-4-(Aminomethyl)cyclohexyl]carbonyl}-4-[2-isopropyl-5-(trifluoromethyl)-1H-benzimidazol-6-yl]-N-(2-oxo-2,3-dihydro-1H-benzimidazol-5-yl)-L-phenylalaninamide hydrochloride

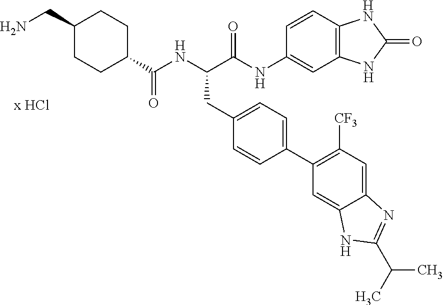 [SMILES: CC(C)c1nc2cc(C(F)(F)F)c(-c3ccc(C[C@H](NC(=O)[C@H]4CC[C@H](CN)CC4)C(=O)Nc4ccc5[nH]c(=O)[nH]c5c4)cc3)cc2[nH]1]

A solution of N-alpha-[(trans-4-{[(tert-butoxycarbonyl)amino]methyl}cyclohexyl)carbonyl]-4-[2-isopropyl-5-(trifluoromethyl)-1H-benzimidazol-6-yl]-N-(2-oxo-2,3-dihydro-1H-benzimidazol-5-yl)-L-phenylalaninamide (18.3 mg, 0.024 mmol) in dioxane (1 ml) was admixed with 4M hydrogen chloride in 1,4-dioxane (0.06 ml, 0.24 mmol) and stirred at RT for 3 days. The precipitated solid was filtered off and washed with dioxane/acetonitrile. The solid was dried under high vacuum. This gave 14.2 mg (85% of theory) of the title compound.

¹H NMR (400 MHz, DMSO-d₆): δ=ppm 0.91 (br. s., 2H), 1.06-1.20 (m, 1H), 1.21-1.34 (m, 1H), 1.46 (s, 3H), 1.47 (s, 3H), 1.50 (br. s., 2H), 1.77 (d, 3H), 2.05-2.19 (m, 1H), 2.64 (t, 2H), 2.94 (dd, 1H), 3.13 (dd, 1H), 3.42-3.50 (m, 1H), 4.69-4.80 (m, 1H), 6.85 (d, 1H), 7.06 (dd, 1H), 7.24 (d, 2H), 7.38 (d, 2H), 7.47 (s, 1H), 7.54 (s, 1H), 7.84 (br. s., 3H), 8.09 (s, 1H), 8.18 (d, 1H), 10.06 (s, 1H), 10.50 (s, 1H), 10.57 (s, 1H).

LC-MS (Method 1): Rₜ=0.61 min; MS (ESIneg): m/z=660 [M−H−HCl]⁻.

### Example 32

### N-alpha-{[trans-4-(Aminomethyl)cyclohexyl]carbonyl}-N-1H-indazol-6-yl-4-(2-isopropyl-3-oxo-2,3-dihydro-1H-indazol-6-yl)-L-phenylalaninamide hydrochloride

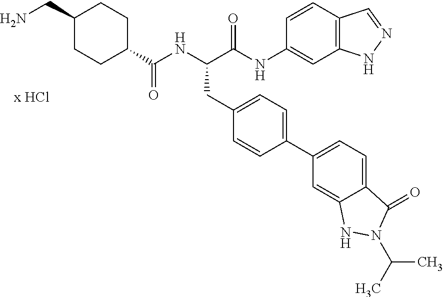 [SMILES: CC(C)n1[nH]c2cc(-c3ccc(C[C@H](NC(=O)[C@H]4CC[C@H](CN)CC4)C(=O)Nc4ccc5cn[nH]c5c4)cc3)ccc2c1=O]

A solution of tert-butyl 6-{4-[(2S)-2-{[(trans-4-{[(tert-butoxycarbonyl)amino]methyl}-cyclohexyl)carbonyl]amino}-3-(1H-indazol-6-ylamino)-3-oxopropyl]phenyl}-2-isopropyl-3-oxo-2,3-dihydro-1H-indazole-1-carboxylate (15.8 mg, 0.02 mmol) in dioxane (1 ml) was admixed with 4M hydrogen chloride in 1,4-dioxane (0.05 ml, 0.20 mmol) and stirred at RT for 6 days. The solvent was removed on a rotary evaporator and the residue was dissolved in DMSO/acetonitrile (about 3 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 6.2 mg (45% of theory) of the title compound.

¹H NMR (400 MHz, DMSO-d₆): δ=ppm 0.83-1.00 (m, 2H), 1.10-1.28 (m, 2H), 1.30 (s, 3H), 1.32 (s, 3H), 1.46 (br. s, 1H), 1.55-1.65 (m, 1H), 1.67-1.83 (m, 3H), 2.11-2.22 (m, 1H), 2.59-2.68 (m, 2H), 2.96 (dd, 1H), 3.13 (dd, 1H), 4.55-4.67 (m, 1H), 4.70-4.82 (m, 1H), 7.15 (d, 1H), 7.35 (dd, 1H), 7.44 (m, 3H), 7.63 (d, 2H), 7.68 (d, 2H), 7.74-7.92 (m, 3H), 7.98 (s, 1H), 8.14 (s, 1H), 8.24 (d, 1H), 10.15 (br. s, 1H), 10.37 (s, 1H).

LC-MS (Method 1): Rₜ=0.60 min; MS (ESIneg): m/z=592 [M−H−HCl]⁻.

### Example 33

### N-alpha-{[trans-4-(Aminomethyl)cyclohexyl]carbonyl}-4-(2-isopropyl-3-oxo-2,3-dihydro-1H-indazol-6-yl)-N-(2-oxo-2,3-dihydro-1H-benzimidazol-5-yl)-L-phenylalaninamide hydrochloride

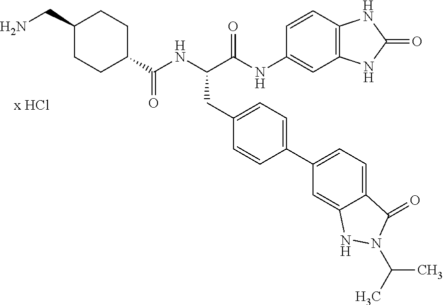 [SMILES: CC(C)n1[nH]c2cc(-c3ccc(C[C@H](NC(=O)[C@H]4CC[C@H](CN)CC4)C(=O)Nc4ccc5[nH]c(=O)[nH]c5c4)cc3)ccc2c1=O]

A solution of tert-butyl 6-(4-{(2S)-2-{[(trans-4-{[(tert-butoxycarbonyl)amino]methyl}-cyclohexyl)carbonyl]amino}-3-oxo-3-[(2-oxo-2,3-dihydro-1H-benzimidazol-5-yl)amino]propyl}-phenyl)-2-isopropyl-3-oxo-2,3-dihydro-1H-indazole-1-carboxylate (45.7 mg, 0.056 mmol) in dioxane (2 ml) was admixed with 4M hydrogen chloride in 1,4-dioxane (0.14 ml, 0.56 mmol) and stirred at RT for 5 days. The solvent was removed on a rotary evaporator and the residue was dissolved in methanol (about 3 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 24.3 mg (67% of theory) of the title compound.

¹H NMR (400 MHz, DMSO-d₆): δ=ppm 0.84-0.99 (m, 2H), 1.10-1.29 (m, 2H), 1.30 (s, 3H), 1.32 (s, 3H), 1.42-1.51 (m, 1H), 1.58 (d, 1H), 1.67-1.85 (m, 3H), 2.07-2.21 (m, 1H), 2.62 (t, 2H), 2.88-2.98 (m, 1H), 3.06-3.14 (m, 1H), 4.55-4.63 (m, 1H), 4.64-4.73 (m, 1H), 6.84 (d, 1H), 7.05 (dd, 1H), 7.35 (dd, 1H), 7.39-7.48 (m, 4H), 7.62 (d, 2H), 7.68 (d, 1H), 7.85 (br. s., 3H), 8.18 (d, 1H), 10.06 (s, 1H), 10.21 (br. s, 1H), 10.50 (s, 1H), 10.56 (s, 1H).

LC-MS (Method 1): Rₜ=0.56 min; MS (ESIneg): m/z=608 [M−H−HCl]⁻.

### Example 34

### N-alpha-{[trans-4-(Aminomethyl)cyclohexyl]carbonyl}-4-(2-isopropyl-5-methyl-3-oxo-2,3-dihydro-1H-indazol-6-yl)-N-(2-oxo-2,3-dihydro-1H-benzimidazol-5-yl)-L-phenylalaninamide hydrochloride

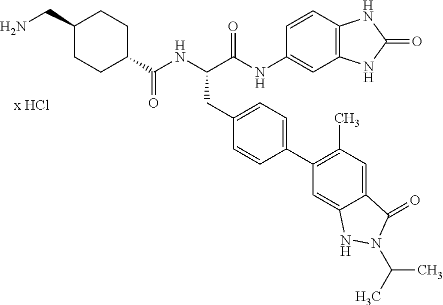 [SMILES: Cc1cc2c(=O)n(C(C)C)[nH]c2cc1-c1ccc(C[C@H](NC(=O)[C@H]2CC[C@H](CN)CC2)C(=O)Nc2ccc3[nH]c(=O)[nH]c3c2)cc1]

A solution of N-alpha-[(trans-4-{[(tert-butoxycarbonyl)amino]methyl}cyclohexyl)carbonyl]-4-(2-isopropyl-5-methyl-3-oxo-2,3-dihydro-1H-indazol-6-yl)-N-(2-oxo-2,3-dihydro-1H-benzimidazol-5-yl)-L-phenylalaninamide (66 mg, 0.091 mmol) in dioxane (2 ml) was admixed with 4M hydrogen chloride in 1,4-dioxane (0.34 ml, 1.37 mmol) and stirred at RT overnight. The precipitated solid was filtered off and washed with dioxane/acetonitrile. The solid was dried under high vacuum. This gave 59 mg (95% of theory) of the title compound.

¹H NMR (400 MHz, DMSO-d₆): δ=ppm 0.82-1.00 (m, 2H), 1.08-1.20 (m, 1H), 1.22-1.32 (m, 1H), 1.28 (s, 3H), 1.30 (s, 3H), 1.41-1.49 (m, 1H), 1.55 (d, 1H), 1.65-1.81 (m, 3H), 2.16 (s, 4H), 2.59-2.69 (m, 2H), 2.92 (dd, 1H), 3.11 (dd, 1H), 4.53-4.64 (m, 1H), 4.67-4.78 (m, 1H), 6.84 (d, 1H), 6.97 (s, 1H), 7.04 (dd, 1H), 7.25 (d, 2H), 7.38 (d, 2H), 7.44 (d, 1H), 7.51 (s, 1H), 7.80 (br. s, 3H), 8.16 (d, 1H), 9.85 (br. s, 1H), 10.02 (s, 1H), 10.49 (s, 1H), 10.57 (s, 1H).

LC-MS (Method 1): Rₜ=0.61 min; MS (ESIneg): m/z=622 [M−H−HCl]⁻.

### Example 35

### N-alpha-{[trans-4-(Aminomethyl)cyclohexyl]carbonyl}-4-(4,5-difluoro-2-isopropyl-1H-benzimidazol-6-yl)-N-(2-oxo-2,3-dihydro-1H-benzimidazol-5-yl)-L-phenylalaninamide hydrochloride

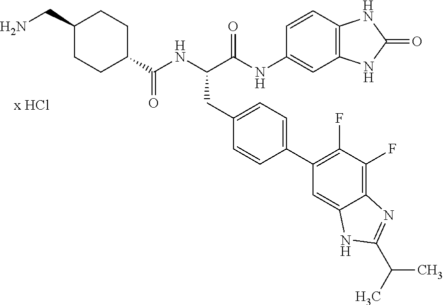 [SMILES: CC(C)c1nc2c(F)c(F)c(-c3ccc(C[C@H](NC(=O)[C@H]4CC[C@H](CN)CC4)C(=O)Nc4ccc5[nH]c(=O)[nH]c5c4)cc3)cc2[nH]1]

A solution of N-alpha-[(trans-4-{[(tert-butoxycarbonyl)amino]methyl}cyclohexyl)carbonyl]-4-(4,5-difluoro-2-isopropyl-1H-benzimidazol-6-yl)-N-(2-oxo-2,3-dihydro-1H-benzimidazol-5-yl)-L-phenylalaninamide (48 mg, 0.055 mmol) in dioxane (1.5 ml) was admixed with 4M hydrogen chloride in 1,4-dioxane (0.21 ml, 0.83 mmol) and stirred at RT overnight. The solvent was removed on a rotary evaporator and the residue was dissolved in DMSO/acetonitrile (about 3 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 30 mg (81% of theory) of the title compound.

¹H NMR (400 MHz, DMSO-d₆): δ=ppm 0.85-1.00 (m, 2H), 1.13-1.33 (m, 2H), 1.35 (s, 3H), 1.37 (s, 3H), 1.41-1.52 (m, 1H), 1.60 (d, 1H), 1.68-1.81 (m, 3H), 2.07-2.21 (m, 1H), 2.61-2.69 (m, 2H), 2.92 (dd, 1H), 3.10 (dd, 1H), 3.15-3.23 (m, 1H), 4.65-4.75 (m, 1H), 6.84 (d, 1H), 7.02 (dd, 1H), 7.28 (d, 1H), 7.37-7.51 (m, 5H), 7.65 (br. s, 3H), 8.13 (d, 1H), 9.99 (s, 1H), 10.49 (s, 1H), 10.57 (s, 1H).

LC-MS (Method 1): Rₜ=0.60 min; MS (ESIneg): m/z=628 [M−H−HCl]⁻.

### Example 36

### N-alpha-{[trans-4-(Aminomethyl)cyclohexyl]carbonyl}-4-(2-cyclopropyl-7-methyl[1,2,4]triazolo[1,5-a]pyridin-6-yl)-N-(2-oxo-2,3-dihydro-1H-benzimidazol-5-yl)-L-phenylalaninamide hydrochloride

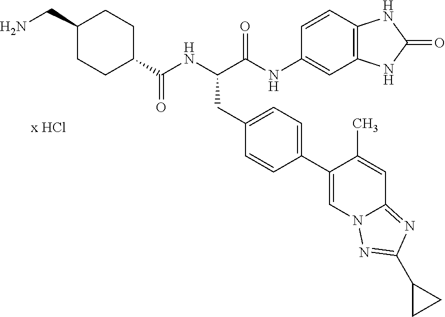 [SMILES: Cc1cc2nc(C3CC3)nn2cc1-c1ccc(C[C@H](NC(=O)[C@H]2CC[C@H](CN)CC2)C(=O)Nc2ccc3[nH]c(=O)[nH]c3c2)cc1]

A solution of N-alpha-[(trans-4-{[(tert-butoxycarbonyl)amino]methyl}cyclohexyl)carbonyl]-4-(2-cyclopropyl-7-methyl[1,2,4]triazolo[1,5-a]pyridin-6-yl)-N-(2-oxo -2,3-dihydro-1H-benzimidazol-5-yl)-L-phenylalaninamide (63 mg, 0.089 mmol) in dioxane (2 ml) was admixed with 4M hydrogen chloride in 1,4-dioxane (0.33 ml, 0.134 mmol) and stirred at RT overnight. The precipitated solid was filtered off and washed with dioxane/acetonitrile. The solid was dried under high vacuum. This gave 56 mg (98% of theory) of the title compound.

¹H NMR (400 MHz, DMSO-d₆): δ=ppm 0.85-0.95 (m, 2H), 0.99 (dd, 2H), 1.07 (dt, 2H), 1.11-1.33 (m, 2H), 1.42-1.53 (m, 1H), 1.53-1.62 (m, 1H), 1.55-1.56 (m, 1H), 1.67-1.82 (m, 3H), 2.15 (d, 2H), 2.24 (s, 3H), 2.63 (br. s., 2H), 2.94 (dd, 1H), 3.11 (dd, 1H), 4.63-4.76 (m, 1H), 6.84 (d, 1H), 7.04 (dd, 1H), 7.34 (d, 2H), 7.39-7.46 (m, 3H), 7.62 (s, 1H), 7.83 (br. s., 3H), 8.22 (s, 1H), 8.61 (s, 1H), 10.04 (s, 1H), 10.50 (s, 1H), 10.56 (s, 1H).

LC-MS (Method 1): Rₜ=0.60 min; MS (ESIneg): m/z=605 [M−H−HCl]⁻.

### Example 37

### N-alpha-{[trans-4-(Aminomethyl)cyclohexyl]carbonyl}-4-(2-isopropyl-6-methyl-3H-imidazo[4,5-b]pyridin-5-yl)-N-(2-oxo-2,3-dihydro-1H-benzimidazol-5-yl)-L-phenylalaninamide hydrochloride

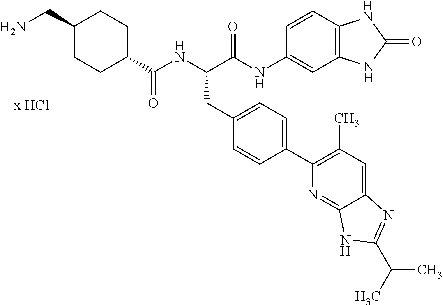 [SMILES: Cc1cc2nc(C(C)C)[nH]c2nc1-c1ccc(C[C@H](NC(=O)[C@H]2CC[C@H](CN)CC2)C(=O)Nc2ccc3[nH]c(=O)[nH]c3c2)cc1]

A solution of N-alpha-[(trans-4-{[(tert-butoxycarbonyl)amino]methyl}cyclohexyl)carbonyl]-4-(2-isopropyl-6-methyl-3H-imidazo[4,5-b]pyridin-5-yl)-N-(2-oxo-2,3-dihydro-1H-benzimidazol-5-yl)-L-phenylalaninamide (87 mg, 0.12 mmol) in dioxane (2 ml) was admixed with 4M hydrogen chloride in 1,4-dioxane (0.31 ml, 1.23 mmol) and stirred at RT for 6 days. Subsequently, the solution was admixed once again with 4M hydrogen chloride in 1,4-dioxane (0.2 ml, 0.8 mmol) and stirred at RT for a further 3 days. The solvent was removed on a rotary evaporator and the residue was dissolved in DMSO/acetonitrile (about 3 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 55 mg (66% of theory) of the title compound.

¹H NMR (400 MHz, DMSO-d₆): δ=ppm 0.83-1.01 (m, 2H), 1.12-1.36 (m, 2H), 1.45 (s, 3H), 1.46 (s, 3H), 1.48-1.53 (m, 1H), 1.56-1.65 (m, 1H), 1.69-1.83 (m, 3H), 2.10-2.21 (m, 1H), 2.41 (s, 3H), 2.59-2.68 (m, 2H), 2.96 (dd, 1H), 3.13 (dd, 1H), 3.41-3.48 (m, 1H), 4.66-4.78 (m, 1H), 6.83 (d, 1H), 7.04 (dd, 1H), 7.40-7.45 (m, 3H), 7.46-7.50 (m, 2H), 7.84 (br. s, 3H), 8.15 (s, 1H), 8.21 (d, 1H), 10.04 (s, 1H), 10.48 (s, 1H), 10.56 (s, 1H).

LC-MS (Method 1): Rₜ=0.60 min; MS (ESIneg): m/z=607 [M−H−HCl]⁻.

### Example 38

### N-alpha-{[trans-4-(Aminomethyl)cyclohexyl]carbonyl}-4-(2-isopropyl-6-methyl-1H-benzimidazol-5-yl)-N-(2-oxo-2,3-dihydro-1H-benzimidazol-5-yl)-D-phenylalaninamide hydrochloride (Enantiomer 1)

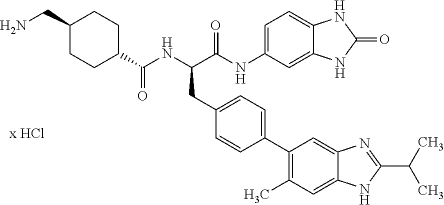

N-alpha-[(trans-4-{[(tert-Butoxycarbonyl)amino]methyl}cyclohexyl)carbonyl]-4-(2-isopropyl-6-methyl-1H-benzimidazol-5-yl)-N-(2-oxo-2,3-dihydro-1H-benzimidazol-5-yl)-D-phenylalaninamide (enantiomer 1) (16 mg, 0.023 mmol) was suspended in dichloromethane (1 ml) and admixed with 4M hydrogen chloride in dioxane (8.5 μl, 33.9 μmol). The reaction mixture was stirred at RT overnight, admixed with further 4M hydrogen chloride in dioxane (8.5 μl, 33.9 μmol), stirred at RT for 2 h until conversion was complete and concentrated to dryness under reduced pressure. This gave 12 mg (81% of theory) of the title compound.

¹H NMR (400 MHz, DMSO-d₆): δ=ppm 0.78-0.99 (m, 3H), 1.08-1.20 (m, 1H), 1.24-1.30 (m, 1H), 1.45 (d, 7H), 1.51-1.58 (m, 1H), 1.68-1.80 (m, 3H), 2.09-2.19 (m, 1H), 2.27 (s, 3H), 2.59-2.65 (m, 2H), 2.92 (dd, 1H), 3.11 (dd, 1H), 3.42-3.50 (m, 1H), 4.66-4.76 (m, 1H), 6.80-6.86 (m, 1H), 6.99-7.05 (m, 1H), 7.22-7.27 (m, 2H), 7.35-7.40 (m, 2H), 7.43 (s, 2H), 7.59-7.66 (m, 1H), 7.80 (br. s, 3H), 8.13-8.20 (m, 1H), 9.88-10.05 (m, 1H), 10.44-10.49 (m, 1H), 10.52-10.58 (m, 1H).

LC-MS (Method 4): Rₜ=0.62 min; MS (ESIpos): m/z=608.5 [M+H−HCl]⁺.

Specific optical rotation (P2000 polarimeter): [a]=−39.8°+/−0.83° (c=1.51 mg/ml, methanol, 20° C., 589 nm).

### Example 39

### N-alpha-{[trans-4-(Aminomethyl)cyclohexyl]carbonyl}-4-(2-isopropyl-6-methyl-1H-benzimidazol-5-yl)-N-(2-oxo-2,3-dihydro-1H-benzimidazol-5-yl)-L-phenylalaninamide hydrochloride (Enantiomer 2)

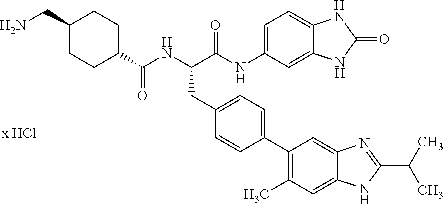

N-alpha-[(trans-4-{[(tert-Butoxycarbonyl)amino]methyl}cyclohexyl)carbonyl]-4-(2-isopropyl-6-methyl-1H-benzimidazol-5-yl)-N-(2-oxo-2,3-dihydro-1H-benzimidazol-5-yl)-L-phenylalaninamide (enantiomer 2) (18 mg, 0.025 mmol) was suspended in dichloromethane (1 ml) and admixed with 4M hydrogen chloride in dioxane (32 μl, 127.2 μmol). The reaction mixture was stirred at RT overnight, admixed with further 4M hydrogen chloride in dioxane (16 μl, 63.6 μmol), and stirred at RT for 2 h until conversion was complete. The reaction mixture was admixed with acetonitrile, and the precipitate was filtered off and dried under reduced pressure. This gave 14 mg (83% of theory) of the title compound.

¹H NMR (400 MHz, DMSO-d₆): δ=ppm 0.78-0.99 (m, 3H), 1.08-1.20 (m, 1H), 1.24-1.30 (m, 1H), 1.45 (d, 7H), 1.51-1.58 (m, 1H), 1.68-1.80 (m, 3H), 2.09-2.19 (m, 1H), 2.27 (s, 3H), 2.59-2.65 (m, 2H), 2.92 (dd, 1H), 3.11 (dd, 1H), 3.42-3.50 (m, 1H), 4.66-4.76 (m, 1H), 6.80-6.86 (m, 1H), 6.99-7.05 (m, 1H), 7.22-7.27 (m, 2H), 7.35-7.40 (m, 2H), 7.43 (s, 2H), 7.59-7.66 (m, 1H), 7.80 (br. s, 3H), 8.13-8.20 (m, 1H), 9.88-10.05 (m, 1H), 10.44-10.49 (m, 1H), 10.52-10.58 (m, 1H).

LC-MS (Method 4): Rₜ=0.61 min; MS (ESIpos): m/z=608.5 [M+H−HCl]⁺.

Specific optical rotation (P2000 polarimeter): [a]=40.0°+/−1.26° (c=1.52 mg/ml, methanol, 20° C., 589 nm).

B) Assessment of Physiological Efficacy

The suitability of the inventive compounds for treatment of thromboembolic or hyperfibrinolytic disorders can be demonstrated in the following assay systems:

a) Test Descriptions (In Vitro)

a.1) Measurement of FXIa Inhibition

To determine the factor XIa inhibition of the substances according to the invention, a biochemical test system is used which utilizes the reaction of a peptidic factor XIa substrate to determine the enzymatic activity of human factor XIa. Here, factor XIa cleaves from the peptic factor XIa substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions are placed into the wells of white microtitre plates from Greiner (384 wells). Subsequently, the following are added successively: 20 μl of assay buffer (50 mmol/l Tris buffer pH 7.4; 100 mmol/l sodium chloride; 5 mmol/l calcium chloride; 0.1% bovine serum albumin) and 20 μl of factor XIa from Kordia (0.45 nM in assay buffer). After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulphoxide instead of test substance in dimethyl sulphoxide), and IC50 values are calculated from the concentration/activity relationships. Activity data from this test are listed in Table A below:

TABLE A

| Example | | | |
|---|---|---|---|
| No. | IC₅₀ [nM] | Example No. | IC₅₀ [nM] |
| 1 | 0.9 | 2 | 2.6 |
| 3 | 1.4 | 4 | 5.6 |
| 5 | 5.1 | 6 | 9.0 |
| 7 | 4.6 | 8 | 6.0 |
| 9 | 3.7 | 10 | 1.9 |
| 11 | 3.2 | 12 | 2.4 |
| 13 | 6.6 | 14 | 6.8 |
| 15 | 3.8 | 16 | 3.2 |
| 17 | 0.5 | 18 | 2.5 |
| 19 | 3.1 | 20 | 0.9 |
| 21 | 0.7 | 22 | 0.5 |
| 23 | 2.3 | 24 | 0.5 |
| 25 | 13 | 26 | 1.4 |
| 27 | 0.9 | 28 | 37 |
| 29 | 3.9 | 30 | 1.7 |
| 31 | 3.1 | 32 | 28 |
| 33 | 17 | 34 | 1.5 |
| 35 | 6.9 | 36 | 3.6 |
| 37 | 4.9 | 38 | 37 |
| 39 | 1.1 | | |

a.2) Determination of the Selectivity

To demonstrate the selectivity of the substances with respect to FXIa inhibition, the test substances are examined for their inhibition of other human serin proteases, such as factor Xa, trypsin and plasmin. To determine the enzymatic activity of factor Xa (1.3 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of sodium chloride, 0.1% BSA [bovine serum albumin], 5 mmol/l of calcium chloride, pH 7.4) and incubated for 15 min with test substance in various concentrations in dimethyl sulphoxide and also with dimethyl sulphoxide without test substance. The enzymatic reaction is then started by addition of the appropriate substrates (5 μmol/l of Boc-Ile-Glu-Gly-Arg-AMC from Bachem for factor Xa and trypsin, 50 μmol/l of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulphoxide instead of test substance in dimethyl sulphoxide) and IC50 values are calculated from the concentration/activity relationships.

a.3) Thrombin Generation Assay (Thrombogram)

The effect of the test substances on the thrombogram (thrombin generation assay according to Hemker) is determined in vitro in human plasma (Octaplas® from Octapharma).

In the thrombin generation assay according to Hemker, the activity of thrombin in coagulating plasma is determined by measuring the fluorescent cleavage products of the substrate 1-1140 (Z-Gly-Gly-Arg-AMC, Bachem). The reactions are carried out in the presence of varying concentrations of test substance or the corresponding solvent. To start the reaction, reagents from Thrombinoscope (30 pM or 0.1 pM recombinant tissue factor, 24 μM phospholipids in HEPES) are used. Moreover, a thrombin calibrator from Thrombinoscope is used whose amidolytic activity is required for calculating the thrombin activity in a sample containing an unknown amount of thrombin. The test is carried out according to the specifications of the manufacturer (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM HEPES, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser.

Using the Thrombinoscope software, the thrombogram is calculated and represented graphically. The following parameters are calculated: lag time, time to peak, peak, ETP (endogenous thrombin potential) and start tail.

a.4) Determination of the Anticoagulatory Activity

The anticoagulatory activity of the test substances is determined in vitro in human and animal plasma (for example mouse, rat, rabbit, pig and dog plasma). To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

The activated partial thromboplastin time (aPTT) is determined in the presence of varying concentrations of test substance or the corresponding solvent using a commercial test kit (C.K. Prest from Diagnostica Stago). The test compounds are incubated with the plasma and the PTT reagent (cephalin, kaolin) at 37° C. for 3 minutes. Coagulation is then started by addition of a 25 mM aqueous calcium chloride solution, and the time when coagulation occurs is determined. The concentration of test substance which brings about a 1.5-fold extension of the aPTT is determined Activity data from this test are listed in Table B below:

TABLE B

| | aPTT | | |
|---|---|---|---|
| Example No. | [μmol/l] | Example No. | aPTT [μmol/l] |
| 1 | 0.07 | 3 | 0.14 |
| 4 | 0.24 | 5 | 0.06 |
| 6 | 0.41 | 7 | 0.28 |
| 8 | 0.06 | 9 | 0.07 |
| 10 | 0.07 | 11 | 0.08 |
| 12 | 0.18 | 13 | 0.18 |
| 14 | 0.23 | 15 | 0.27 |
| 16 | 0.27 | 17 | 0.28 |
| 18 | 0.3 | 19 | 0.1 |
| 20 | 0.26 | 21 | 0.3 |
| 22 | 0.18 | 23 | 0.27 |
| 25 | 0.96 | 26 | 0.18 |
| 27 | 0.16 | 28 | 1.42 |
| 29 | 0.28 | 30 | 0.07 |
| 31 | 0.07 | 32 | 0.43 |
| 33 | 0.1 | 34 | 0.11 |
| 35 | 0.54 | 36 | 0.08 |
| 37 | 0.06 | | |

a.5) Determination of Fibrinolytic Activity

Antifibrinolytic activity in vitro is assessed in human, platelet-free plasma. Tissue factor (TF) (1 pM) and tissue plasminogen activator (tPA) (40 nM) are pipetted into plasma together with 12.5 mM aqueous calcium chloride solution and substance. On occurrence of clotting, the subsequent clot lysis is determined photometrically over a period of 30 minutes.

a.6) Measurement of Plasmin Inhibition

The plasmin inhibition of the inventive substances is determined using a biochemical test system which utilizes the reaction of a peptidic plasmin substrate to determine the enzymatic activity of human plasmin. Here, plasmin cleaves from the peptic plasmin substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions are placed into the wells of white microtitre plates from Greiner (384 wells). Subsequently, the following are added successively: 20 μl of assay buffer (50 mmol/l Tris buffer pH 7.4; 100 mmol/l sodium chloride; 5 mmol/l calcium chloride; 0.1% bovine serum albumin) and 20 μl of plasmin from Kordia (0.3 μg/ml in assay buffer). After 15 min of incubation, the enzyme reaction is started by addition of 20 μl of the plasmin substrate MeOSuc-Ala-Phe-Lys-AMC dissolved in assay buffer (150 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulphoxide instead of test substance in dimethyl sulphoxide), and ICso values are calculated from the concentration/activity relationships. Activity data from this test are listed in Table C below:

TABLE C

| Example | | | |
|---|---|---|---|
| No. | IC₅₀ [nM] | Example No. | IC₅₀ [nM] |
| 1 | 22 | 8 | 21 |
| 9 | 25 | 12 | 5.9 |
| 19 | 1.3 | 20 | 0.4 |
| 21 | 0.4 | 22 | 0.6 |
| 23 | 0.95 | 24 | 1.4 |
| 25 | 1.7 | 26 | 2.7 |
| 27 | 0.45 | 30 | 5.9 |
| 31 | 2.1 | 32 | 8.7 |
| 33 | 5.9 | 34 | 1.7 |
| 35 | 1.5 | 36 | 1.2 |
| 37 | 0.5 | 38 | 20 |
| 39 | 0.64 | | |

b) Determination of Antithrombotic Activity (In Vivo)

b.1) Arterial Thrombosis Model (iron(II) Chloride-Induced Thrombosis) in Combination with Ear Bleeding Time in Rabbits

The antithrombotic activity of the FXIa inhibitors is tested in an arterial thrombosis model. Thrombus formation is triggered here by causing chemical injury to a region in the carotid artery in rabbits. Simultaneously, the ear bleeding time is determined.

Male rabbits (Crl:KBL (NZW)BR, Charles River) receiving a normal diet and having a body weight of 2.2-2.5 kg are anaesthetized by intramuscular administration of xylazine and ketamine (Rompun, Bayer, 5 mg/kg and Ketavet, Pharmacia & Upjohn GmbH, 40 mg/kg body weight). Anaesthesia is furthermore maintained by intravenous administration of the same preparations (bolus: continuous infusion) via the right auricular vein.

The right carotid artery is exposed and the tissue injury is then caused by wrapping a piece of filter paper (10 mm×10 mm) on a Parafilm® strip (25 mm×12 mm) around the carotid artery without disturbing the blood flow. The filter paper contains 100 μl of a 13% strength solution of iron(II) chloride (Sigma) in water. After 5 min, the filter paper is removed and the vessel is rinsed twice with aqueous 0.9% strength sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h, respectively, before the injury.

Ear bleeding time is determined 2 min after injury to the carotid artery. To this end, the left ear is shaved and a defined 3-mm-long incision (blade Art. Number 10-150-10, Martin, Tuttlingen, Germany) is made parallel to the longitudinal axis of the ear. Care is taken here not to damage any visible vessels. Any blood that extravasates is taken up in 15 second intervals using accurately weighed filter paper pieces, without touching the wound directly. Bleeding time is calculated as the time from making the incision to the point in time where no more blood can be detected on the filter paper. The volume of the extravasated blood is calculated after weighing of the filter paper pieces.

c) Determination of Fibrinolytic Activity (In Vivo)

c.1) Hyper-Fibrinolytic Rats

The determination of antifibrinolytic activity in vivo is conducted in hyperfibrinolytic rats. After anaesthetization and catheterization of the animals, hyperfibrinolysis is triggered by infusion of tissue plasminogen activator (tPA) (8 mg/kg/h). 10 minutes after commencement of tPA infusion, the substances are administered as an i.v. bolus. After a further 15 minutes, tPA infusion is ended and a transsection of the tail is conducted. Subaqual bleeding (in physiological saline at 37° C.) is observed over 30 minutes and the bleed time is determined.

C) Working Examples of Pharmaceutical Formulations

The inventive substances can be converted to pharmaceutical formulations, for example, as follows:

Tablet:

Composition:

100 mg of the compound of Example 1, 50 mg of lactose (monohydrate), 50 mg of maize starch, 10 mg of polyvinylpyrrolidone (PVP) and 2 mg of magnesium stearate.

Tablet weight 212 mg, diameter 8 mm, radius of curvature 12 mm.

Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tablet press (see above for format of the tablet).

Oral Suspension:

Composition:

1000 mg of the compound of Example 1, 1000 mg of ethanol (96%), 400 mg of Rhodigel and 99 g of water.

A single dose of 100 mg of the compound according to the invention corresponds to 10 ml of oral suspension.

Production:

The Rhodigel is suspended in ethanol, and the compound of Example 1 is added to the suspension. The water is added while stirring. The mixture is stirred for about 6 h until swelling of the Rhodigel is complete.

Solution for Oral Administration:

Composition:

500 mg of the inventive compound, 2.5 g of polysorbate and 97 g of polyethylene glycol 400. A single dose of 100 mg of the inventive compound corresponds to 20 g of oral solution.

Production:

The compound according to the invention is suspended in the mixture of polyethylene glycol and polysorbate while stirring. The stirring operation is continued until dissolution of the compound according to the invention is complete.

I.V. Solution:

The inventive compound is dissolved in a concentration below the saturation solubility in a physiologically acceptable solvent (e.g. isotonic saline, glucose solution 5% and/or polyethylene glycol 400/water 30% m/m). The solution is subjected to sterile filtration and dispensed into sterile and pyrogen-free injection vessels.

1. A compound of the formula 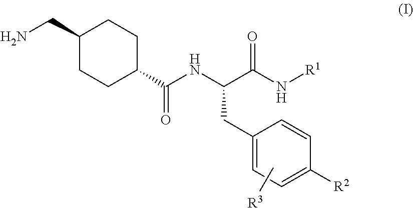
in which
R¹ is a group of the formula 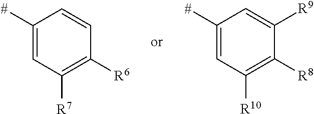
where # is the attachment site to the nitrogen atom,
R⁶ is 5-membered heteroaryl,
where heteroaryl may be substituted by a substituent selected from the group consisting of oxo, chlorine, cyano, hydroxyl and C₁-C₃-alkyl,
in which alkyl may be substituted by 1 to 3 substituents selected independently from the group consisting of hydroxyl, amino, hydroxycarbonyl and methoxy, or
in which alkyl may be substituted by 1 to 7 fluorine substituents, or
in which alkyl is substituted by a substituent selected from the group consisting of hydroxyl, amino, hydroxycarbonyl and methoxy, and in which alkyl is additionally substituted by 1 to 6 fluorine substituents,
R⁷ is hydrogen, fluorine or chlorine,
R⁸ and R⁹ together with the carbon atoms to which they are bonded form a 5-membered heterocycle,
where the heterocycle may be substituted by 1 to 2 substituents selected independently from the group consisting of oxo, chlorine, cyano, hydroxyl, C₁-C₃-alkyl, pyrazolyl and pyridyl,
in which alkyl may be substituted by 1 to 3 substituents selected independently from the group consisting of hydroxyl, amino, hydroxycarbonyl and methoxy, or
in which alkyl may be substituted by 1 to 7 fluorine substituents, or
in which alkyl is substituted by a substituent selected from the group consisting of hydroxyl, amino, hydroxycarbonyl and methoxy, and
in which alkyl is additionally substituted by 1 to 6 fluorine substituents,
R¹⁰ is hydrogen, fluorine or chlorine,
R² is 9- or 10-membered bicyclic heteroaryl,
where heteroaryl may be substituted by 1 to 3 substituents selected independently from the group consisting of oxo, fluorine, chlorine, cyano, trifluoromethyl, hydroxyl, amino, C₁-C₃-alkylamino, C₁-C₃-alkyl and C₃-C₆-cycloalkyl,
in which alkyl may be substituted by a substituent selected from the group consisting of amino and C₁-C₃-alkylamino, or
R² is a group of the formula 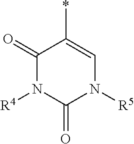
where * is the attachment site to the phenyl ring,
R⁴ is hydrogen, C₁-C₄-alkyl or benzyl,
R⁵ is hydrogen, C₁-C₄-alkyl or benzyl,
R³ is hydrogen, fluorine, chlorine, methyl or methoxy,
or one of the salts thereof, solvates thereof or solvates of the salts thereof.

2. The compound of claim 1, characterized in that
R¹ is a group of the formula 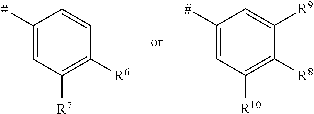
where # is the attachment site to the nitrogen atom,
R⁶ is 5-membered heteroaryl,
R⁷ is hydrogen or fluorine,
R⁸ and R⁹ together with the carbon atoms to which they are bonded form a 5-membered heterocycle,
where the heterocycle may be substituted by an oxo substituent,
R¹⁰ is hydrogen,
R² is 9- or 10-membered bicyclic heteroaryl,
where heteroaryl may be substituted by 1 to 2 substituents selected independently from the group consisting of oxo, amino, C₁-C₃-alkyl, cyclopropyl and cyclobutyl,
in which alkyl may be substituted by an amino substituent,
R³ is hydrogen,
or one of the salts thereof, solvates thereof or solvates of the salts thereof.

3. The compound of claim 1, characterized in that
R¹ is a group of the formula 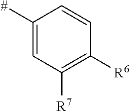
where # is the attachment site to the nitrogen atom,
R⁶ is tetrazolyl,
R⁷ is hydrogen or fluorine, or
R¹ is 2,3-dihydro-1H-indazol -6-yl, 2,3-dihydro-1H-benzimidazol-5-yl, 1H-benzimidazol-6-yl or 1H-indazol-6-yl,
where 2,3-dihydro-1H-indazol-6-yl and 2,3-dihydro-1H-benzimidazol-5-yl may be substituted by an oxo substituent,
R² is benzimidazolyl, indazolyl, pyrrolopyridinyl, isoquinolinyl, tetrahydroquinolinyl, 1H-imidazo[4,5-b]pyridin-6-yl, 1,2,3,4-tetrahydropyrido[2,3-b]pyrazin-7-yl, 2,3-dihydro-1H-isoindol-5-yl, 2,3-dihydro-1H-indazol-6-yl, [1,2,4]triazolo-[1,5-a]pyridin-6-yl or 3H-imidazo[4,5-b]pyridin-5-yl,
where benzimidazolyl, indazolyl, pyrrolopyridinyl, isoquinolinyl, tetrahydroquinolinyl, 1H-imidazo[4,5-b]pyridin-6-yl, 1,2,3,4-tetrahydropyrido[2,3-b]pyrazin-7-yl, 2,3-dihydro-1H-isoindol-5-yl, 2,3-dihydro-1H-indazol-6-yl, [1,2,4]triazolo-[1,5-a]pyridin-6-yl and 3H-imidazo[4,5-b]pyridin-5-yl may be substituted by 1 to 2 substituents selected independently from the group consisting of oxo, amino, methyl, ethyl, n-propyl, isopropyl, cyclopropyl and cyclobutyl, in which ethyl, n-propyl and isopropyl may be substituted by an amino substituent,
R³ is hydrogen,
or one of the salts thereof, solvates thereof or solvates of the salts thereof.

4. A method for preparing the compound of claim 1 of the formula (I) or one of the salts thereof, solvates thereof or solvates of the salts thereof, characterized in that a compound of the formula 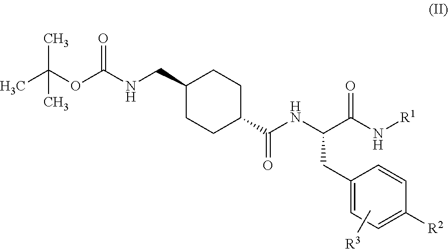
in which R¹, R² and R³ are each as defined in claim 1, is reacted with an acid.

5. A method for treatment and/or prophylaxis of diseases using the compound of claim 1.

6. A method of making a medicament for treatment and/or prophylaxis of diseases using the compound of claim 1.

7. A method of making a medicament for the treatment and/or prophylaxis of thrombotic or thromboembolic disorders or severe perioperative blood loss using the compound of claim 1.

8. A medicament comprising the compound of claim 1 in combination with an inert, nontoxic, pharmaceutically suitable excipient.

9. A method for treatment and/or prophylaxis of thrombotic or thromboembolic disorders or severe perioperative blood loss using the medicament of claim 8.

10. A method for treating thrombotic or thromboembolic disorders or severe perioperative blood loss in man and animals by administration of a therapeutically effective amount of at least one compound according to claim 1.

11. A method for treating thrombotic or thromboembolic disorders or severe perioperative blood loss in man and animals by administration of a therapeutically effective amount of at least one compound according to the medicament of claim 8.

12. A method for treating thrombotic or thromboembolic disorders or severe perioperative blood loss in man and animals by administration of a therapeutically effective amount of at least one compound according to the medicament of claim 6.
